(19) United States
(12) Patent Application Publication
(10) Pub. No.: US 2013/0157884 A1
(43) Pub. Date: Jun. 20, 2013

(54) METHODS AND COMPOSITIONS INVOLVING MIRNA EXPRESSION LEVELS FOR DISTINGUISHING PANCREATIC CYSTS

(71) Applicant: Asuragen, Inc., Austin, TX (US)

(72) Inventors: Alex T. Adai, Austin, TX (US); Anna E. Szafranska-Schwarzbach, Austin, TX (US); Bemard F. Andruss, Austin, TX (US); Anirban Maitra, Baltimore, MD (US); Hanno Matthaei, Bonn, DE (US); Dennis Wylie, Austin, TX (US); Ralph Hruban, Baltimore, MD (US)

(73) Assignee: Asuragen, Inc., Austin, TX (US)

(21) Appl. No.: 13/662,212

(22) Filed: Oct. 26, 2012

Related U.S. Application Data
(60) Provisional application No. 61/551,874, filed on Oct. 26, 2011.
(60) Provisional application No. 61/552,340, filed on Oct. 27, 2011.

(51) Int. Cl. C12Q 1/68; G06F 19/00

(52) U.S. Cl. CPC C12Q 1/6837; G06F 19/3431
USPC 506/9; 435/6.12; 702/19

(57) ABSTRACT

Embodiments concern methods and compositions for evaluating a pancreatic cyst in a patient based on the expression levels of one or more miRNAs to determine whether the pancreatic cyst is a low or high risk lesion and in further need of treatment such as resection.

This application claims priority to U.S. Provisional Patent Application 61/551,874 filed on Oct. 26, 2011 and to U.S. Provisional Patent Application 61/552,340 filed on Oct. 27, 2011, both of which are hereby incorporated by reference in their entirety.

This invention was made with government support under grant P50CA0624292 from the National Cancer Institute and grant R44CA118785 from the National Institutes of Health/National Cancer Institute. The government has certain rights in the invention.

### BACKGROUND OF THE INVENTION

I. Field of the Invention

The invention relates generally to the field of molecular biology. More particularly, it concerns methods and compositions involving microRNA molecules (miRNAs). Certain aspects of the invention include applications for miRNAs in diagnostics, therapeutics, and prognostics for pancreatic cancer and other pancreatic lesions.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and arise from longer precursors, which are transcribed from non-protein-encoding genes. See review of Carrington et al. (2003). The precursors form structures that fold back on themselves in self-complementary regions; they are then processed by the nuclease Dicer in animals or DCL1 in plants. miRNA molecules interrupt translation through precise or imprecise base-pairing with their targets.

Many miRNAs are conserved among diverse organisms, and this has led to the suggestion that miRNAs are involved in essential biological processes throughout the life span of an organism (Esquela-Kerscher and Slack, 2006). In particular, miRNAs have been implicated in regulating cell growth and cell and tissue differentiation, cellular processes that are associated with the development of cancer. For instance, lin-4 and let-7 both regulate passage from one larval state to another during C. elegans development (Ambros, 2001). miR-14 and bantam are Drosophila miRNAs that regulate cell death, apparently by regulating the expression of genes involved in apoptosis (Brennecke et al., 2003, Xu et al., 2003).

Research on miRNAs is increasing as scientists are beginning to appreciate the broad role that these molecules play in the regulation of eukaryotic gene expression. In particular, several recent studies have shown that expression levels of numerous miRNAs are associated with various cancers (reviewed in Esquela-Kerscher and Slack, 2006). Reduced expression of two miRNAs correlates strongly with chronic lymphocytic leukemia in humans, providing a possible link between miRNAs and cancer (Calin et al., 2002). Others have evaluated the expression patterns of large numbers of miRNAs in multiple human cancers and observed differential expression of almost all miRNAs across numerous cancer types (Lu et al., 2005).

Pancreatic cancer is a particularly challenging disease to diagnose and treat. In 2010 about 43,140 people in the United States are diagnosed with adenocarcinoma of the pancreas, and about 36,000 people die each year from pancreatic cancer (Jemal et al., 2010). Pancreatic carcinoma ranks as the fourth leading cause of cancer deaths in the United States, and the five year survival rate (˜4%) is the lowest among all cancers (Jemal et al., 2006).

Most pancreatic cancers are adenocarcinomas of the ductal epithelium (Freelove and Walling, 2006)—or pancreatic ductal adenocarcinomas (PDAC). PDAC is characterized by its late clinical presentation, early and aggressive local invasion and high metastatic potential. The lack of sensitive early detection strategies and its strong resistance to chemotherapy and radiation therapy compounds the overall very poor prognosis of PDAC, which has a median survival time following diagnosis of 3-5 months. Currently, effective diagnostic methods and/or treatments for pancreatic cancer are lacking (Monti et al., 2004). Surgery is still the only effective treatment option, improving the median survival time to 10-20 months; however, at the time of diagnosis only 20% of PDACs are amenable to surgery and cure is rarely achieved (See Yeo et al., 2002). Thus, improved early diagnosis modalities as well as new therapeutic targets for the development of effective treatment strategies are urgently needed to improve the dismal prognosis of PDAC.

mRNAs have been implicated in the development of pancreatic cancer and can serve as biomarkers for the disease (Habbe et al., 2009; Ryu et al., 2010; Gironella et al., 2007; Gottardo et al., 2007; Ali et al., 2010; Dillhoff et al., 2008; Ryu et al., 2011).

There has been a dramatic increase in the diagnosis of cysts of the pancreas, largely owing to the widespread use of high-resolution imaging technologies (Matthaei and Maitra, 2011; DiMagno, 2007). Studies suggest that approximately one fourth of the normal population has a pancreatic cyst (Kimura et al., 1995). While most of these lesions are benign, do not cause any symptoms, and may be managed conservatively, some represent precursors of pancreatic cancer. The primary goals in the management of these cysts are to prevent harmful surgery in patients with benign cysts (if asymptomatic), and to identify patients for whom a surgical resection is unavoidable. In cases of suspected precancerous or malignant cystic lesions, the individual decision for or against surgery is a significant challenge (Roggin et al., 2010; Winter et al., 2006).

The most frequently observed cystic precursor lesion of PDAC is an intraductal papillary mucinous neoplasm (IPMN). Despite a growing awareness of IPMNs, their clinical management still requires substantial improvement. For example, it is currently not possible to predict the presence of an IPMN without resection and precise histopathologic examination of the resected pancreatic cyst. This diagnostic uncertainty hampers attempts to analyze and learn from the clinical behavior of especially small and asymptomatic suspected IPMNs during prospective studies. This dilemma led to the establishment of consensus guidelines (“Sendai guidelines” or “Tanaka guidelines”) proposed by an American Pancreatic Association (APA) consortium, which enabled a more systematic management of suspected IPMNs. These recommendations were based on a handful of large observational studies focused primarily on preoperative imaging and a patient's clinical history (Tanaka et al., 2006). However, because these guidelines rely on indirect clinical (symptoms) and radiologic (cyst size, presence of mural nodules in the cyst wall) parameters, they have limitations. For example, suspected branch-duct IPMNs are frequently hard to differentiate from mucinous cystic neoplasms (MCNs), and serous lesions pose a diagnostic and therapeutic challenge in interdisciplinary tumor boards (Tanaka, 2011).

The appropriate management of pancreatic cysts mandates surgical resection in cases of associated malignancy or a likely malignant transformation. Equally, benign cysts must be recognized and treated conservatively whenever the patient is asymptomatic, because pancreatic surgery is associated with a high morbidity and mortality (Winter et al., 2006).

Biomarkers that predict malignant potential would be helpful for the successful management of IPMNs. A recent study determined that about two thirds of all IPMNs harbored mutations in the GNAS gene at codon 201 (Wu et al., 2011). DNA alterations, such as loss of heterozygosity at characteristic genomic locations and KRAS2 gene mutations, may help predict the grade of dysplasia in an IPMN and the presence of an invasive carcinoma associated with a cyst (Khalid et al., 2005; Schoedel et al., 2006).

Other studies have sought to identify diagnostic targets in pancreatic cyst fluid (Khalid et al., 2005; Khalid et al., 2009; Ryu et al., 2004; Allen et al., 2009). For example, high levels of the Carcinoembryonic Antigen (CEA) protein have been used predict the presence of a mucinous cyst (Allen et al., 2009; Nagula et al., 2010). Unfortunately, however, CEA is not capable of predicting the presence of an associated invasive carcinoma and is relatively non-specific due to its frequent elevation in benign lesions. Such false-positive results may lead to inappropriate treatment (Nagula et al., 2010; Raval et al., 2010).

With increasing numbers of pancreatic cysts being detected each year, methods and compositions that improve the ability to distinguish between benign IPMNs and higher risk lesions are needed.

### SUMMARY OF THE INVENTION

Disclosed herein are methods for evaluating a pancreatic cyst in a patient, which provides a clinician with information useful for diagnosis and/or treatment options. Methods involve obtaining information about the levels of expression of certain microRNAs or miRNAs whose expression levels differ in different types of pancreatic cysts. In some embodiments, differences in miRNA expression between or among different pancreatic cysts are highlighted when expression level differences are first compared among two or more miRNAs and those differential values are compared to or contrasted with the differential values of one or more different types of pancreatic cysts or normal pancreatic cells. Embodiments concern methods and compositions that can be used for evaluating a pancreatic cyst, differentiating a pancreatic cyst, distinguishing pancreatic cysts, identifying a pancreatic cyst as a high risk lesion, identifying a pancreatic cyst as a low risk lesion, identifying a pancreatic cyst as a target for surgical resection, determining a pancreatic cyst should not be surgically resected, categorizing a pancreatic cyst, diagnosing a pancreatic cyst or pancreatic cysts, providing a prognosis to a patient regarding a pancreatic cyst or pancreatic cysts, evaluating treatment options for a pancreatic cyst, or treating a patient with a pancreatic cyst. These methods can be implemented involving steps and compositions described below in different embodiments.

In some embodiments, methods involve measuring from a pancreatic cyst sample from the patient the level of expression of at least two of the following miRNAs: miR-18a, miR-24, miR-30a-3p, miR-92a, miR-99b, miR-106b, miR-142-3p, miR-342-3p, or miR-532-3p. In certain embodiments, the level of expression of 1, 2, 3, 4, 5, 6, 7, 8, or 9 of the following miRNAs, which may or may not be a diff pair miRNA, may be measured: miR-18a, miR-24, miR-30a-3p, miR-92a, miR-99b, miR-106b, miR-142-3p, miR-342-3p, or miR-532-3p. In certain embodiments, methods involve measuring from a pancreatic cyst sample from the patient the level of expression of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA. The term “diff pair miRNA” refers to a miRNA that is one member of a pair of miRNAs where the expression level of one miRNA of the diff pair in a sample is compared to the expression level of the other miRNA of the diff pair in the same sample. The expression levels of two diff pair miRNAs may be evaluated with respect to each other, i.e., compared, which includes but is not limited to subtracting, dividing, multiplying or adding values representing the expression levels of the two diff pair miRNAs. The term “biomarker miRNA” refers to a miRNA whose expression level is indicative of a particular disease or condition. A biomarker miRNA may be a diff pair miRNA in certain embodiments. As part of a diff pair, the level of expression of a biomarker miRNA may highlight or emphasize differences in miRNA expression between different populations, such as low or high risk pancreatic lesions. In some embodiments, when miRNA expression is different in a particular population relative to another population, differences between miRNA expression levels can be increased, highlighted, emphasized, or otherwise more readily observed in the context of a diff pair.

Measuring a microRNA or miRNA refers to measuring the amount of a mature microRNA or miRNA, though it is contemplated that a mature miRNA may be indirectly determined by measuring the level of an immature or unprocessed form of the miRNA, such as the double-stranded RNA molecule or RNA hairpin structure. Moreover, in some embodiments, amount of a mature miRNA is determined by measuring the amount of one or more of the miRNA's target or the targets complement. An miRNA's target refers to the endogenous RNA in the pancreas cell that is the target for the miRNA and whose expression is affected by the miRNA. Consequently, any embodiments discussed herein in the context of determining the amount of a microRNA (i.e., the mature form of a microRNA) can be implemented instead by measuring a precursor of the miRNA or one or more of the miRNA's target (or the complement thereof). Unless qualified, the term “measuring” refers to directly measuring. Mature miRNAs may be indirectly determined by directly measuring precursor microRNA molecules.

In some embodiments described herein, the disease or condition is the type or category of pancreatic cyst, such as whether the cyst is a high risk or low risk lesion. While embodiments have application with non-human mammals and their corresponding miRNAs, human patients and human microRNAs are the focus of embodiments described herein. In additional embodiments, it is contemplated that methods may involve determining the level of 1, 2, 3, 4, 5, 6, 7, 8, 9, 10, 11 or 12 different miRNAs or at least 1, 2, 3, 4, 5, 6, 7, 8, 9, 10, 11, or 12 different miRNAs, and any range derivable therein. Methods may or may not involve determining the amount or level of expression of a non-miRNA nucleic acid in sample.

Measuring or assaying for expression levels of a microRNA can be accomplished by a variety of different chemical and/or enzymatic reactions that are well known to those of skill in the art. In certain embodiments, methods may involve amplification and/or hybridization. It is contemplated that the level of a mature microRNA (miRNA) may be indirectly determined by measuring the level of the immature or unprocessed microRNA. Whether the mature or immature form of a microRNA is measured depends on the detection method, such as which primer or probe is used in the method. A person of ordinary skill in the art knows how this would be implemented.

In further embodiments, methods involve comparing levels of expression of different miRNAs in the pancreatic cyst sample to each other or to expression levels of other biomarkers, which occurs after a level of expression is measured or obtained. In certain embodiments, miRNA expression levels are compared to each other. In certain embodiments, methods include comparing the measured level of expression of each biomarker miRNA to the level of expression of another biomarker miRNA. In some embodiments, methods involve comparing the level of expression of the at least one biomarker miRNA to the level of expression of a comparative microRNA to determine a biomarker diff pair value. A “comparative miRNA” refers to a miRNA whose expression level is used to evaluate the level of another miRNA in the sample; in some embodiments, the expression level of a comparative microRNA is used to evaluate a biomarker miRNA expression level. For example, a differential value between the biomarker miRNA and the comparative miRNA can be calculated or determined or evaluated; this value is a number that is referred to as a “diff pair value” when it is based on the expression level of two miRNAs. A diff pair value can be calculated, determined or evaluated using one or more mathematical formulas or algorithms. In some embodiments, the value is calculated, determined or evaluated using computer software. Moreover, it is readily apparent that the miRNA used as a biomarker and the miRNA used as the comparative miRNA may be switched, and that any calculated value can be evaluated accordingly by a person of ordinary skill in the art. However, a person of ordinary skill in the art understands that different pair analysis may be adjusted, particular with respect to altering the comparative miRNA in a pair without affecting the concept of the embodiments discussed herein. In certain embodiments, the expression level of a biomarker miRNA that is being compared to or the expression level of a comparative miRNA may be a normalized expression level and/or that biomarker miRNA or comparative miRNA may be considered a normalizer. It is contemplated that a person of ordinary skill in the art will recognize that different classifiers can be generated using the data provided herein to make the determinations and evaluations discussed herein.

A comparative miRNA may be any miRNA, but in some embodiments, the comparative miRNA is chosen because it allows a statistically significant and/or relatively large difference in expression to be detected or highlighted between expression levels of the biomarker in one pancreatic cyst population as compared to a different pancreatic cyst population. Furthermore, a particular comparative miRNA in a diff pair may serve to increase any difference observed between diff pair values of different pancreated cyst populations, for example, a high risk cyst population compared to a low risk cyst population. In further embodiments, the comparative miRNA expression level serves as an internal control for expression levels. In some embodiments, the comparative miRNA is one that allows the relative or differential level of expression of a biomarker miRNA to be distinguishable from the relative or differential level of expression of that same biomarker in a different pancreatic cyst population. In some embodiments, the expression level of a comparative miRNA is a normalized level of expression for the different pancreatic cyst populations, while in other embodiments, the comparative miRNA level is not normalized.

It is contemplated that any of the following miRNAs may be considered or treated as a biomarker microRNA: miR-18a, miR-24, miR-30a-3p, miR-92a, miR-99b, miR-106b, miR-142-3p, miR-342-3p, or miR-532-3p. It is further contemplated that any of the following miRNAs may be treated or considered as a comparative microRNA: miR-18a, miR-24, miR-30a-3p, miR-92a, miR-99b, miR-106b, miR-142-3p, miR-342-3p, or miR-532-3p. In some embodiments, at least or at most 1, 2, 3, 4, 5, 6, 7, 8, or 9 of the following miRNAs is a biomarker miRNA: miR-18a, miR-24, miR-30a-3p, miR-92a, miR-99b, miR-106b, miR-142-3p, miR-342-3p, or miR-532-3p. In other embodiments, at least or at most 1, 2, 3, 4, 5, 6, 7, 8, or 9 of the following miRNAs is a comparative miRNA: miR-18a, miR-24, miR-30a-3p, miR-92a, miR-99b, miR-106b, miR-142-3p, miR-342-3p, or miR-532-3p (it is understood that the same miRNA is not both the biomarker miRNA and the comparative miRNA).

In certain embodiments, there are methods for evaluating a pancreatic cyst in a patient comprising: a) measuring from a pancreatic cyst sample from the patient the level of expression of at least two of the following biomarker miRNAs: miR-24, miR-30a-3p, miR-92a, miR-18a, miR-342-3p, miR-99b, miR-106b, miR-142-3p, or miR-532-3p; and b) calculating a diagnostic score that indicates the probability the pancreatic cyst is a low risk or high risk lesion, wherein the diagnostic score is based on comparisons between the expression levels of the biomarker miRNAs to the expression level of at least one other biomarker miRNA.

In specific embodiments, methods for evaluating a pancreatic cyst in a patient comprise: a) measuring from a pancreatic cyst sample from the patient the level of expression of at least two of the following biomarker miRNAs: miR-24, miR-30a-3p, miR-92a, miR-18a, miR-342-3p, miR-99b, miR-106b, miR-142-3p, or miR-532-3; b) comparing the level of expression each biomarker miRNA to the level of expression of another biomarker miRNA; and, c) calculating a diagnostic score that indicates the probability the pancreatic cyst is a low risk or high risk lesion, wherein the diagnostic score is based on comparisons between the expression levels of the biomarker miRNAs to the expression level of at least one other biomarker miRNA.

In some embodiments, methods involve determining diff pair values after measuring and comparing the level of expression of miRNAs, where the diff pair values are calculated from 1, 2, 3, 4, 5, 6, or 7 (or any range therein) or at least 1, 2, 3, 4, 5, 6, or 7 (or any range therein) of the following diff pairs: miR-24/miR-30a-3p; miR-18a/miR-92a; miR-24/miR-342-3p; miR-24/miR-99b; miR-106b/miR-92a; miR-142-3p/miR-92a; or miR-30a-3p/miR-532-3p. In additional embodiments, methods may also involve evaluating the sample based on the biomarker diff pair value, wherein the biomarker diff pair value indicates whether the pancreatic cyst is a low risk or high risk lesion. The term “low risk lesion” refers to a lesion that is not generally considered malignant or aggressive, and this includes, but is not limited to, serous cystadenoma (SCA), low grade intraductal papillary mucinous neoplasm (LG-IPMN), pseudocysts, and branch duct IPMNs; low risk lesions tend to be managed conservatively by watchful waiting instead of surgical resection. The term “high risk lesion” refers to a lesion that may progress into an invasive or non-invasive cancer, and this includes, but is not limited to mucinous cystic neoplasm (MCN), solid pseudopapillary neoplasm (SPN), neuroendocrine tumor (NET), high grade intraductal papillary mucinous neoplasm (HG-IPMN), and main duct IPMNs; high risk lesions tend to managed by surgical resection with a curative intent. In certain embodiments, instead of qualifying or determining that a pancreatic cyst has a particular risk of being a high or low risk lesion, a pancreatic cyst may be qualified or evaluated with respect to its being one of the specific categories or subcategories discussed above, such as SCA, MCN, SPN, NET, IPMN, or a type of IPMN. In certain embodiments methods involve identifying the pancreatic cyst as a low risk or high risk lesion or categorizing a pancreatic cyst as being SCA, MCN, SPN, NET, IPMN, or a type of IPMN.

In other embodiments, there may be a series of evaluations performed on a sample, For instance, in some embodiments, the cyst may first undergo cytological examination or evaluation prior to implementing any molecular tests.

In some embodiments, methods will involve determining or calculating a diagnostic or risk score based on data concerning the expression level of one or more miRNAs, meaning that the expression level of the one or more miRNAs is at least one of the factors on which the score is based. A diagnostic score will provide information about the biological sample, such as the general probability that the pancreatic cyst is a high risk lesion, that the pancreatic cyst is a low risk lesion, or both. In some embodiments, the diagnostic score represents the probability that the pancreatic cyst is more likely than not either a high risk lesion or a low risk lesion. In certain embodiments, a probability value is expressed as a numerical integer or number that represents a probability of 0% likelihood to 100% likelihood that a patient has a particular category of lesion, such as a high risk or low risk lesion. In some embodiments, the probability value is expressed as a numerical integer or number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has a particular category of lesion. Alternatively, the probability may be expressed generally in percentiles, quartiles, or deciles.

In some embodiments, methods include evaluating one or more differential pair values using a scoring algorithm to generate a diagnostic score for a high risk or low risk pancreatic lesion, wherein the patient is identified as having or as not having such a based on the score. It is understood by those of skill in the art that the score is a predictive value about whether the patient does or does not such a lesion. In some embodiments, a report is generated and/or provided that identifies the diagnostic score or the values that factor into such a score. In some embodiments, a cut-off score is employed to characterize a sample as likely having a high risk lesion or not having a high risk lesion (or alternatively a low risk lesion). In some embodiments, the risk score for the patient is compared to a cut-off score to characterize the biological sample from the patient with respect to high risk or low risk pancreatic lesions. In some embodiments, the score may be assigned to a certain level of risk such as at least or about a 50, 55, 60, 65, 70, 75, 80, 85, 90, 95, or 100% (and any range derivable therein) chance that the cyst is a high risk lesion or that the cyst is a low risk lesion.

In certain embodiments, the weight or significance of diff pair values is not the same when calculating a diagnostic score or doing a risk determination. In some embodiments, the diff pair value for miR-24/miR-30a-3p is weighted more heavily or given more significance than the diff pair value for 1, 2, 3, 4, 5 or all 6 (or any range derivable therein) of the following diff pair values: miR-18a/miR-92a; miR-24/miR-342-3p; miR-24/miR-99b; miR-106b/miR-92a; miR-142-3p/miR-92a; or miR-30a-3p/miR-532-3p. In some embodiments, the diff pair value for miR-18a/miR-92a is weighted more heavily or given more significance than the diff pair value for 1, 2, 3, 4, 5 or all 6 (or any range derivable therein) of the following diff pair values: miR-24/miR-30a-3p; miR-24/miR-342-3p; miR-24/miR-99b; miR-106b/miR-92a; miR-142-3p/miR-92a; or miR-30a-3p/miR-532-3p. In some embodiments, the diff pair value for miR-24/miR-342-3p is weighted more heavily or given more significance than the diff pair value for 1, 2, 3, 4, 5 or all 6 (or any range derivable therein) of the following diff pair values: miR-24/miR-30a-3p; miR-18a/miR-92a; miR-24/miR-99b; miR-106b/miR-92a; miR-142-3p/miR-92a; or miR-30a-3p/miR-532-3p. In some embodiments, the diff pair value for miR-24/miR-99b is weighted more heavily or given more significance than the diff pair value for 1, 2, 3, 4, 5 or all 6 (or any range derivable therein) of the following diff pair values: miR-24/miR-30a-3p; miR-18a/miR-92 a; miR-24/miR-342-3p; miR-106b/miR-92a; miR-142-3p/miR-92a; or miR-30a-3p/miR-532-3p. In some embodiments, the diff pair value for miR-106b/miR-92a is weighted more heavily or given more significance than the diff pair value for 1, 2, 3, 4, 5 or all 6 (or any range derivable therein) of the following diff pair values: miR-24/miR-30a-3p; miR-18a/miR-92a; miR-24/miR-342-3p; miR-24/miR-99b; miR-142-3p/miR-92a; or miR-30a-3p/miR-532-3p. In some embodiments, the diff pair value for miR-142-3p/miR-92a is weighted more heavily or given more significance than the diff pair value for 1, 2, 3, 4, 5 or all 6 (or any range derivable therein) of the following diff pair values: miR-24/miR-30a-3p; miR-18a/miR-92a; miR-24/miR-342-3p; miR-24/miR-99b; miR-106b/miR-92a; or miR-30a-3p/miR-532-3p. In some embodiments, the diff pair value for miR-30a-3p/miR-532-3p is weighted more heavily or given more significance than the diff pair value for 1, 2, 3, 4, 5 or all 6 (or any range derivable therein) of the following diff pair values: miR-24/miR-30a-3p; miR-18a/miR-92a; miR-24/miR-342-3p; miR-24/miR-99b; miR-106b/miR-92a; or miR-142-3p/miR-92a. A weighted coefficient may be applied to one or more diff pair values in some embodiments.

A difference between or among weighted coefficients or between or among the weights of the diff pair values may be, be at least or be at most 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 times or -fold (or any range derivable therein).

In some embodiments, determination of calculation of a diagnostic score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain embodiments, the diagnostic score is calculated using one or more statistically significantly differentially expressed biomarkers (either individually or as diff pairs).

In certain embodiments, there are methods for evaluating a pancreatic cyst in a patient comprising: a) measuring from a pancreatic cyst sample from the patient the level of expression of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA; b) comparing the level of expression of the at least one biomarker miRNA to the level of expression of a comparative microRNA to determine a biomarker diff pair value; and, c) evaluating the sample based on the biomarker diff pair value, wherein the biomarker diff pair value indicates whether the pancreatic cyst is a low risk or high risk lesion.

In certain methods, 1, 2, 3, 4, 5, 6, or all 7 or at least 1, 2, 3, 4, 5, 6, or 7 of the following diff pairs is evaluated: miR-24/miR-30a-3p; miR-18a/miR-92a; miR-24/miR-342-3p; miR-24/miR-99b; miR-106b/miR-92a ; miR-142-3p/miR-92a; or miR-30a-3p/miR-532-3p. In additional methods steps include measuring the level of expression of at least one of the following miRNAs: miR-15a, miR-16, miR-21, miR-17-5p, miR-100, miR-107, miR-155, miR-181a, miR-181 c, miR-210, miR-221, or miR-223. In certain embodiments, 1, 2, 3, 4, 5, 6, 7, 8, 9, 10, 11 or 12 or at least or at most 1, 2, 3, 4, 5, 6, 7, 8, 9, 10, 11 or 12 of the following miRNAs is measured or assay for its expression level: miR-15a, miR-16, miR-21, miR-17-5p, miR-100, miR-107, miR-155, miR-181a, miR-181c, miR-210, miR-221, or miR-223.

In other embodiments, a diff pair need not be evaluated and instead, a coefficient value is applied to each miRNA expression level. The coefficient value reflects the weight that the expression level of that particular miRNA has in assessing the chances that a particular pancreatic cyst is a high risk lesion or a low risk lesion. In some embodiments, instead of a diff pair value, a coefficient value is used for the measured expression level of miR-18a, miR-24, miR-30a-3p, miR-92a, miR-99b, miR-106b, miR-142-3p, miR-342-3p, and/or miR-532-3p. In certain embodiments, the coefficient values for a plurality of miRNAs whose expression levels are measured add up to zero (0). Methods and computer readable medium can be implemented with coefficient values instead of or in addition to diff pair values. In some cases, a coefficient may be multiplied against a diff pair value to reflect the weight of that diff pair in any analysis or diagnostic score calculation.

a) receiving information corresponding to a level of expression in a pancreatic cyst sample from a patient of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA; and b) determining a biomarker diff pair value using information corresponding to the at least one biomarker miRNA and information corresponding to the level of expression of a comparative microRNA, the diff pair value being indicative of whether the pancreatic cyst is a low risk or high risk lesion.

In other embodiments, methods are provided for evaluating a pancreatic cyst in a patient comprising: a) measuring from a pancreatic cyst sample from the patient the level of expression of at least miR-30a-3p and at least one comparative miRNA; b) comparing the level of expression between miR-30a-3p and the second microRNA to determine a miR-30-3p diff pair value; and, c) evaluating the sample based on the miR-30-3p diff pair value, wherein the miR-30-3p diff pair value indicates whether the pancreatic cyst is a low risk or high risk lesion. A miR-30-3p diff pair value, for example, refers to a diff pair value in which miR-30-3p is a biomarker miRNA.

In further embodiments there are methods of evaluating a pancreatic cyst from a patient comprising: a) from a sample of the pancreatic cyst, measuring the level of expression of at least miR-24 and at least one of the following comparative microRNAs: miR-30a-3p, miR-342-3p, and miR-99b; b) comparing the level of expression between miR-24 and the at least one comparative microRNA to determine a miR-24 diff pair value; and, c) evaluating the sample based on the miR-24 diff pair value, wherein the miR-24 diff pair value indicates whether the pancreatic cyst is a low risk or high risk lesion.

In other embodiments, there are methods for evaluating a pancreatic cyst from a patient comprising: a) from a sample of the pancreatic cyst, measuring the level of expression of at least miR-92a and at least one of the following second microRNAs selected from the group consisting of miR-106b and miR-142-3p; b) comparing the level of expression between miR-92a and the second microRNA to determine a miR-92a diff pair value; and, c) evaluating the sample based on the miR-92a diff pair value, wherein the miR-92a diff pair value is indicative of whether the pancreatic cyst is a high risk lesion or a low risk lesion.

Additional embodiments concern methods for evaluating a pancreatic cyst sample from a patient comprising a) from the sample, measuring the level of expression of at least miR-18a and at least a second microRNA; b) comparing the level of expression between miR-18a and the second microRNA to determine a miR-18a diff pair value; and, c) evaluating the sample based on the miR-18a diff pair value, wherein the miR-18a diff pair value is indicative of whether the pancreatic cyst is a high risk lesion or a low risk lesion. In particular embodiments, the second miRNA is miR-92a.

Further methods are provided for evaluating a pancreatic cyst sample from a patient comprising: a) from the sample, measuring the level of expression of at least miR-106b and at least a second microRNA; b) comparing the level of expression between miR-106b and the second microRNA to determine a miR-106b diff pair value; and, c) evaluating the sample based on the miR-106b diff pair value, wherein the miR-106b diff pair value is indicative of whether the pancreatic cyst is a high risk lesion or a low risk lesion. In certain embodiments, the second microRNA is miR-92a.

More methods are provided for evaluating a pancreatic cyst sample from a patient comprising: a) from the sample, measuring the level of expression of at least miR-142-3p and at least a second microRNA; b) comparing the level of expression between miR-142-3p and the second microRNA to determine a miR-142-3p diff pair value; and, c) evaluating the sample based on the miR-142-3p diff pair value, wherein the miR-142-3p diff pair value is indicative of whether the pancreatic cyst is a high risk lesion or a low risk lesion. In certain embodiments, the second microRNA is miR-92a.

In other embodiments, there are methods for evaluating a pancreatic cyst sample from a patient comprising: a) from the sample, measuring the level of expression of at least miR-342-3p and at least a second microRNA; b) comparing the level of expression between miR-342-3p and the second microRNA to determine a miR-342-3p diff pair value; and, c) evaluating the sample based on the miR-342-3p diff pair value, wherein the miR-342-3p diff pair value is indicative of whether the pancreatic cyst is a high risk lesion or a low risk lesion. In certain embodiments, the second microRNA is miR-24.

Further embodiments concern methods for evaluating a pancreatic cyst sample from a patient comprising: a) from the sample, measuring the level of expression of miRNAs from at least two diff pairs selected from the group consisting of miR-24/miR-30a-3p; miR-18a/miR-92 a; miR-24/miR-342-3p; miR-24/miR-99b; miR-106b/miR-92a; miR-142-3p/miR-92a; and miR-30a-3p/miR-532-3p; b) calculating diff pair values for the at least two of the diff pairs; and, c) determining a diagnostic score for the pancreatic cyst sample.

In some embodiments there are methods for analyzing a pancreatic cyst sample from a patient comprising: a) measuring the level of expression in the cyst sample of at least two of the following biomarker miRNAs: miR-18a, miR-24, miR-30a-3p, miR-92a, miR-99b, miR-106b, miR-142-3p, miR-342-3p, miR-532-3p; b) measuring the level of expression of at least one comparative miRNA, wherein the level of expression of the comparative miRNA is compared with one or more biomarker miRNA expression levels; c) comparing each of the at least two levels of biomarker expression with the level of expression of the at least one comparative RNA to determine a diff pair value; and, d) determining a risk score that indicates the risk for a high risk lesion or a low risk lesion.

Also provided are methods for evaluating a pancreatic cyst in a patient comprising: a) measuring the level of miR-532-3p expression in a pancreatic cyst sample from the patient, and b) determining a diagnostic score for the sample based on the level of miR-532-3p, wherein the diagnostic score indicates the probability that the pancreatic cyst is a high risk lesion or a low risk lesion. In certain embodiments, methods further comprise measuring the level of expression of a second microRNA. In further embodiments, methods comprise comparing the level of expression between miR-532-3p and the second microRNA to determine a miR-532-3p diff pair value; wherein the diagnostic score is based on the miR-532-3p diff pair value.

In additional embodiments, there are methods for evaluating a pancreatic cyst in a patient comprising: a) measuring from a pancreatic cyst sample from the patient the level of expression of at least one of the following miRNAs: miR-18a, miR-24, miR-30a-3p, miR-92a, miR-99b, miR-106b, miR-142-3p, miR-342-3p, or miR-532-3p; b) comparing the level of expression of the at least one miRNA to the level of expression of at least a first comparative microRNA; and, c) evaluating the sample based on the comparison between the level of expression of the at least one miRNA to the level of expression of at least a first comparative microRNA, wherein the evaluation indicates whether the pancreatic cyst is a low risk or high risk lesion.

Some embodiments concern treatment options depending on a determination made about a pancreatic cyst in a patient. In some embodiments, there are methods for treating a patient comprising a) obtaining information regarding at least one diff pair value, wherein the at least one diff pair value relates to the level of expression of one or two of the following miRNAs: miR18a, miR-24, miR-30a-3p, miR-92a, miR-99b, miR-106b, miR-142-3p, miR-342-3p, or miR-532-3p; b) determining the patient as having or likely having a high risk lesion; and, c) resecting a portion of the patient's pancreas in order to remove all or part of the suspected high risk lesion. In some embodiments, a patient is also administered radiation and/or chemotherapy as part of a treatment regimen. In further embodiments, methods may involve determining the patient as having or likely having a low risk lesion, or determining the patient has not having or likely not having a high risk lesion. In such cases, a clinician may then decide not to subject the patient to a resection. In such cases, the patient may continue to be monitored. In certain embodiments, methods involve imaging an unresected pancreatic cyst before and/or after miRNA levels are measured. In further embodiments, the imaging occurs after 1, 2, 3, 4, 5, 6 months following a test that involves measuring one or more miRNA expression levels.

Methods may involve obtaining from the patient a pancreatic cyst sample, which means the sample is obtained directly from the patient. In other embodiments, a patient's pancreatic tissue sample may be obtained from an entity that is not the patient, such as the doctor, clinician, hospital or laboratory (which itself obtained the sample directly from the patient). In certain embodiments, methods involve a pancreatic tissue sample or a pancreatic cyst sample. In particular embodiments, the sample is a tissue sample, while in other embodiments, the sample is a cystic fluid sample. In some cases, methods involve fixing the tissue sample in formalin and embedding it in paraffin prior to measuring the level of expression of one or more miRNAs or diff pair miRNAs in the sample. In additional embodiments, the sample is obtained by fine needle aspirate or FNA. In other embodiments, the sample is retrieved from a biopsy, such as a fine needle aspiration biopsy (FNAB) or a needle aspiration biopsy (NAB).

In some embodiments, a patient is determined to have a pancreatic cyst sample indicative of a high risk lesion. The term “indicative of a high risk lesion” means the data indicate that the patient likely has a high risk lesion, where “likely” means “greater than not.” In other embodiments, the patient is determined to have a pancreatic cyst sample indicative of a low risk lesion. The determination may or may not be based on a diagnostic score that is calculated based on one or more miRNA expression levels or diff pair values. In additional embodiments, methods involve determining a treatment for the patient based on one or more diff pair values. In some embodiments, methods include determining a treatment for the patient based on a calculated diagnostic score. In some embodiments, a patient may be suspected of having pancreatic cancer or precancer. In other embodiments, the patient may have previously had a pancreatic cyst suspected of being a high risk lesion that was then subsequently treated. In other embodiments, the patient has recurring pancreatic cysts. In still further embodiments, the patient has a familial history of pancreatic cysts, particularly pancreatic cysts that are high risk lesions. In some circumstances, a patient also presents with symptoms of a pancreatic cyst or a high risk lesion, such as jaundice, pain in the upper abdomen or significant weight loss in a short amount of time.

Methods may also involve measuring the level of expression of KRAS2 and/or GNAS, which are not microRNAs. The gene products (nucleic acid and/or protein) of one or both of these genes may be measured. Alternatively, methods may involve determining the presence or absence of a mutation in KRAS2 and/or GNAS. In certain embodiments, one or more mutations in codon 12 and/or 13 may be evaluated and/or detected in embodiments.

In certain embodiments, it is specifically contemplated that miR-21 expression levels are not measured and/or are not used in determining a diagnostic score. In other embodiments, one or more of the following miRNAs is specifically not measured and/or evaluated in methods described herein: miR18a, miR-24, miR-30a-3p, miR-92a, miR-99b, miR-106b, miR-142-3p, miR-342-3p, or miR-532-3p.

Some embodiments further involve isolating ribonucleic or RNA from a biological sample. Other steps may or may not include amplifying a nucleic acid in a sample and/or hybridizing one or more probes to an amplified or non-amplified nucleic acid. In certain embodiments, a microarray may be used to measure or assay the level of miRNA expression in a sample.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs that are related to the disclosed methods and compositions, as well as their sequences, are provided herein. The name of the miRNAs that are used in methods and compositions refers to an miRNA that is at least 90% identical to the named miRNA based on its mature sequence listed herein and that is capable of being detected under the conditions described herein using the designated ABI part number for the probe. In most embodiments, the sequence provided herein is the sequence that is being measured in methods described herein. In some methods, a step may involve using a nucleic acid with the sequence comprising or consisting of any of the complements of SEQ ID NOs:1-9 to measure expression of a miRNA in the sample. Alternatively, probes directed to the immature form of these miRNAs may be used, as may be probes directed to the targets of the miRNAs. In some embodiments, a complement of SEQ ID NO:1 (5′-UAAGGUGCAUCUAGUGCAGAUAG′3′) is used to measure expression of naturally occurring miR-18a in a sample. In other embodiments, a complement of SEQ ID NO:2 (5′-UGGCUCAGUUCAGCAGGAACAG-3′) is used to measure expression of naturally occurring miR-24 in a sample. In further embodiments, a complement of SEQ ID NO:3 (5′-CUUUCAGUCGGAUGUUUGCAGC-3′) is used to measure expression of naturally occurring miR-30a-3p. In further embodiments, a complement of SEQ ID NO:4 (5′-UAUUGCACUUGUCCCGGCCUGU-3′) is used to measure expression of naturally occurring miR-92a. In other embodiments, a complement of SEQ ID NO:5 (5′-CACCCGUAGAACCGACCUUGCG-3′) is used to measure expression of naturally occurring miR-99b. In some embodiments, a complement of SEQ ID NO:6 (5′-UAAAGUGCUGACAGUGCAGAU-3′) is used to measure expression of naturally occurring miR-106b. In other embodiments, a complement of SEQ ID NO:7 (5′-UGUAGUGUUUCCUACUUUAUGGA-3′) is used to measure expression of naturally occurring miR142-3p. In additional embodiments, a complement of SEQ ID NO:8 (5′-UCUCACACAGAAAUCGCACCCGU-3′) is used to measure expression of naturally occurring miR-342-3p. In further embodiments, a complement of SEQ ID NO:9 (5′-CCUCCCACACCCAAGGCUUGCA-3′) is used to measure the expression of naturally occurring miR-532-3p. It is contemplated that a probe used in methods may be 85, 86, 87, 88, 89, 90, 91, 92, 93, 94, 95, 96, 97, 98, 99, ro 100% complementary (and any range derivable therein) to any of SEQ ID NOs 1-9 below.

Methods may also involve one or more of the following steps: obtaining a pancreas sample of a patient; preparing the sample to characterize miRNA in the sample (for instance, for hybridization and/or amplification); storing a sample from a patient; assessing the integrity or adequacy of the sample, such as of the nucleic acids; doing a cytology analysis of the sample; staining all or part of the sample using tissue stains; fixing all or part of the sample; freezing all or part of the sample; transporting the sample; providing or being provided one or more images of the sample; visually accessing the sample directly or remotely, such as with telemedicine; measuring the level of expression of at least one biomarker miRNAs; comparing the level of expression of each biomarker miRNA to the level of expression of another biomarker miRNA; calculating a diagnostic score that indicates the probability the thyroid sample is benign or is malignant, wherein the diagnostic score is based on comparisons between the expression levels of the biomarker miRNAs to the expression level of at least one other biomarker miRNA; reporting the diagnostic score or the probablility the pancreas sample is a high risk or low risk lesion; storing information related to the expression levels of measured miRNA or related to comparisons between levels of miRNA or related to calculations from measured and/or compared expression levels or related to diagnostic scores; implementing an algorithm on a computer to calculate values that reflect comparisons between or among expression levels of biomarker miRNAs; implementing an algorithm on a computer to calculate diagnostic scores; diagnosing the patient as having or not having a high risk lesion; diagnosing the patient as having or not having a low risk lesion; categorizing a thyroid sample as a particular subtype or category high risk pancreatic lesions; categorizing a thyroid sample as a particular subtype or category of low risk pancreatic lesions; monitoring a patient's pancreas; and/or, treating the patient for a high risk lesion. It is contemplated that the score may indicate the probability that the sample is a low risk lesion. In other embodiments, the score may indicate the probability that the sample is high risk lesion. It will also be understood that a sample may be tested or evaluated more than one time either at the same time and/or at different times. In some cases, another test is run to obtain a second opinion on the same sample or a different sample from the patient.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to a level of expression in a pancreatic cyst sample from a patient of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA; and b) determining a biomarker diff pair value using information corresponding to the at least one biomarker miRNA and information corresponding to the level of expression of a comparative microRNA, the diff pair value being indicative of whether the pancreatic cyst is a low risk or high risk lesion. In some embodiments, receiving information comprises receiving from a tangible data storage device information corresponding to a level of expression in a pancreatic cyst sample from a patient of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA. In additional embodiments the medium further comprises computer-readable code that, when executed by a computer, causes the computer to perform one or more additional operations comprising: sending information corresponding to the biomarker diff pair value to a tangible data storage device. In specific embodiments, it further comprises computer-readable code that, when executed by a computer, causes the computer to perform one or more additional operations comprising: sending information corresponding to the biomarker diff pair value to a tangible data storage device. In certain embodiments, receiving information comprises receiving from a tangible data storage device information corresponding to a level of expression in a pancreatic cyst sample from a patient of at least two of the following diff pair miRNAs: miR-18a, miR-24, miR-30a-3p, miR-92a, miR-99b, miR-106b, miR-142-3p, miR-342-3p, or miR-532-3p, wherein at least one of the miRNAs is a biomarker miRNA. In even further embodiments, the tangible computer-readable medium has computer-readable code that, when executed by a computer, causes the computer to perform operations further comprising: c) calculating a diagnostic score for the pancreatic sample, wherein the diagnostic score is indicative of the probability that the pancreatic sample is a high risk lesion.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour. In some embodiments, there are systems that implement the processors or that contain the computer readable medium discussed herein for performing the calculations that allow a diagnostic score to be determined.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data.

Also provided are kits containing the disclosed compositions or compositions used to implement the disclosed methods. In some embodiments, kits can be used to evaluate one or more miRNA molecules. In certain embodiments, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more, or any range and combination derivable therein, miRNA probes including those that may specifically hybridize under stringent conditions to miRNAs disclosed herein. In other embodiments, kits or methods may involve 1, 2, 3, 4, 5, 6, 7, 8, 9, 10, 11, or 12 or more (or any range derivable therein) miRNA probes, which may be capable of specifically detecting any of the following miRNAs: miR18a, miR-24, miR-30a-3p, miR-92a, miR-99b, miR-106b, miR-142-3p, miR-342-3p, or miR-532-3p.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any disclosed method or composition, and vice versa. Any embodiment discussed with respect to a particular pancreatic disorder can be applied or implemented with respect to a different pancreatic disorder. Furthermore, the disclosed compositions and kits can be used to achieve the disclosed methods.

Throughout this application, the term “about” is used to indicate that a value includes the standard deviation of error for the device or method being employed to determine the value.

The use of the term “or” in the claims is used to mean “and/or” unless explicitly indicated to refer to alternatives only, or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. However, for a claim using any of these terms, embodiments are also contemplated where the claim is closed and does exclude additional, unrecited elements or method steps.

Other objects, features and advantages of the invention will be apparent from the following detailed description. It should be understood, however, that the detailed description and the specific examples, while indicating specific embodiments, are given by way of illustration only, because various changes and modifications within the spirit and scope of the invention will be apparent to those skilled in the art from this detailed description.

### DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1. Diagram of the study design. Flow chart describing the formalin-fixed, paraffin-embedded (FFPE) tissue study (FTS) and cyst fluid study (CFS) designs, including numbers of miRNA candidates identified in the course of the studies. SCA: serous cystadenoma, LG: low grade, IG: intermediate grade, HG: high grade, IPMN: intraductal papillary mucinous neoplasm, HT: high throughput, NET: neuroendocrine tumor, SPN: solid papillary neoplasm, NAT: normal adjacent tissue.

FIGS. 2A & 2B. Two principal component analysis (PCA) of restricted mean-center normalized Megaplex RT-qPCR data separating samples by diagnoses for both FTS1 (A) and CFS1 (B) specimen sets. Note that one HG IPMN specimen (Disc-CF9) in panel B clusters with the LG IPMN group.

FIGS. 3A & 3B. Performance of logistic regression model. (A) Classification of specimens from the CFS1 and CFS2 re-assigned to training and test sets (line indicates 50% malignancy threshold; symbol key for (B) also applies to (A)). (B) Predictive probability of surgery versus median miRNA Ct of specimens is shown. SRC indicates Spearman Rank Correlation.

FIG. 4. ΔCt values for DiffPairs used as predictors by a logistic model derived from singleplex RT-qPCR data using the CFS1 and CFS2 specimens.

FIG. 5. Mean Cts for all specimens used. (A) Megaplex RT-qPCR data for FTS. (B) and (C) Singleplex RT-qPCR data for FTS. (D) Megaplex RT-qPCR data for CFS. (E) and (F) Singleplex RT-qPCR data for CFS.

FIGS. 6A & 6B. Boxplots showing raw Ct values for Megaplex (A) and for singleplex (B) RT-qPCR expression analyses of FTS1 and FTS1 plus FTS2, respectively.

FIGS. 7A & 7B. Boxplots showing raw Ct values for Megaplex (A) and for singleplex (B) RT-qPCR expression analyses of cyst fluid specimens from the CFS1 and CFS1 plus CFS2, respectively.

FIG. 8. Boxplots of raw Ct values for singleplex RT-qPCR expression analysis of the CFS1 and CFS2 specimens with reassignment to test or training set indicated by separation of panels.

FIG. 9. Raw Ct values of miRNAs involved in DiffPairs used in the logistic regression model, as well as of miR-21, in the CFS1 and CFS2 specimens.

FIG. 10. PCA applied to raw Cts (A) and restricted mean-center normalized Cts (B) CFS1 and CFS2 singleplex RT-qPCR data.

### DETAILED DESCRIPTION OF THE INVENTION

Certain embodiments are directed to compositions and methods relating to preparation and characterization of miRNAs, as well as use of miRNAs for therapeutic, prognostic, and diagnostic applications, particularly those methods and compositions related to assessing and/or identifying pancreatic disease.

### I. mIRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enable them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene. Examples of animal miRNAs include those that imperfectly basepair with the target, which halts translation of the target (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through an RNA-induced silencing complex (RISC) (Denli et al., 2003).

A. Nucleic Acids

In the disclosed compositions and methods miRNAs can be labeled, used in array analysis, or employed in diagnostic, therapeutic, or prognostic applications, particularly those related to pathological conditions of the pancreas. The RNA may have been endogenously produced by a cell, or been synthesized or produced chemically or recombinantly. They may be isolated and/or purified. The term “miRNA,” unless otherwise indicated, refers to the single-stranded processed RNA, after it has been cleaved from its precursor. The name of the miRNA is often abbreviated and referred to without a hsa-, mmu-, or rno-prefix and will be understood as such, depending on the context. Unless otherwise indicated, miRNAs referred to are human sequences identified as miR-X or let-X, where X is a number and/or letter.

In certain experiments, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the World Wide Web at sanger.ac.uk. Moreover, in some embodiments, a miRNA probe is used that does not correspond to a known human miRNA. It is contemplated that these non-human miRNA probes may be used in embodiments or that there may exist a human miRNA that is homologous to the non-human miRNA. While the methods and compositions are not limited to human miRNA, in certain embodiments, miRNA from human cells or a human biological sample is used or evaluated. In other embodiments, any mammalian miRNA or cell, biological sample, or preparation thereof may be employed.

In some embodiments, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNAs are 19-24 nucleotides in length, while miRNA probes are 19-35 nucleotides in length, depending on the length of the processed miRNA and any flanking regions added. miRNA precursors are generally between 62 and 110 nucleotides in human s.

Nucleic acids used in methods and compositions disclosed herein may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000, or any range derivable therein, contiguous nucleotides from any nucleic acid discussed or provided herein, including any miR or precursor miR sequence. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of the sequences disclosed herein. The commonly used name of the miRNA is given (with its identifying source in the prefix, for example, “hsa” for human sequences) and the processed miRNA sequence. Unless otherwise indicated, a miRNA without a prefix will be understood to refer to a human miRNA. A miRNA designated, for example, as miR-1-2 in the application will be understood to refer to hsa-miR-1-2. Moreover, a lowercase letter in the name of a miRNA may or may not be lowercase; for example, hsa-mir-130b can also be referred to as miR-130B. In addition, miRNA sequences with a “mu” or “mmu” sequence will be understood to refer to a mouse miRNA and miRNA sequences with a “rno” sequence will be understood to refer to a rat miRNA. The term “miRNA probe” refers to a nucleic acid probe that can identify a particular miRNA or structurally related miRNAs.

It is understood that a miRNA is derived from genomic sequences or a gene. In this respect, the term “gene” is used for simplicity to refer to the genomic sequence encoding the precursor miRNA for a given miRNA. However, embodiments may involve genomic sequences of a miRNA that are involved in its expression, such as a promoter or other regulatory sequences.

The term “recombinant” generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids can include, can be, or can be at least 60, 65, 70, 75, 80, 85, 90, 95, 96, 97, 98, 99 or 100% complementary to their target. In certain embodiments, the single stranded mature miRNA molecule (typically 17-30 nucleotides in length) is the target of detection.

As used herein, “hybridization”, “hybridizes” or “capable of hybridizing” is understood to mean the forming of a double or triple stranded molecule or a molecule with partial double or triple stranded nature. The term “anneal” is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein, “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of such include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those with a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

A nucleobase may be comprised in a nucleoside or nucleotide, using any chemical or natural synthesis method described herein or known to one of ordinary skill in the art. Such a nucleobase may be labeled or may be part of a molecule that is labeled and contains the nucleobase.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods disclosed herein. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside, and nucleotide analogs or derivatives are well known in the art, and have been described (see for example, Scheit, 1980, incorporated herein by reference).

Additional non-limiting examples of nucleosides, nucleotides, or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acid probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake, and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′ deoxyguanosine nucleotides and nucleic acid analogs thereof).

5. Modified Nucleotides

Labeling methods and kits may use nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the methods and compositions are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in some embodiments are alkyl-linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, etc. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in some embodiments. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 84(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased.

Methods may involve the use of organic solvents and/or alcohol to isolate nucleic acids, particularly miRNA used in methods and compositions disclosed herein. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column may work particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

### II. LABELS AND LABELING TECHNIQUES

In some embodiments, miRNAs are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In particular embodiments, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide(s) is catalytically added to a miRNA, and the unlabeled nucleotide is modified with a chemical moiety that enables it to be subsequently labeled. In some embodiments, the chemical moiety is a reactive amine such that the nucleotide is an amine-modified nucleotide. Examples of amine-modified nucleotides are well known to those of skill in the art, many being commercially available.

In contrast to labeling of cDNA during its synthesis, the issue for labeling miRNA is how to label the already existing molecule. Some aspects concern the use of an enzyme capable of using a di- or tri-phosphate ribonucleotide or deoxyribonucleotide as a substrate for its addition to a miRNA. Moreover, in specific embodiments, a modified di- or tri-phosphate ribonucleotide is added to the 3′ end of a miRNA. The source of the enzyme is not limiting. Examples of sources for the enzymes include yeast, gram-negative bacteria such as E. coli, lactococcus lactis, and sheep pox virus.

Enzymes capable of adding such nucleotides include, but are not limited to, poly(A) polymerase, terminal transferase, and polynucleotide phosphorylase. In specific embodiments, a ligase is contemplated as not being the enzyme used to add the label, and instead, a non-ligase enzyme is employed.

Terminal transferase may catalyze the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides include Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides include Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP.

It is contemplated that nucleic acids may be labeled with two different labels. Furthermore, fluorescence resonance energy transfer (FRET) may be employed in disclosed methods (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and other ligands, include ligands for an antibody.

C. Visualization Techniques

A number of techniques for visualizing or detecting labeled nucleic acids are readily available. Such techniques include, microscopy, arrays, fluorometry, light cyclers or other real time PCR machines, FACS analysis, scintillation counters, phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acids. Furthermore, a person of ordinary skill in the art is well aware of ways of visualizing, identifying, and characterizing labeled nucleic acids, and accordingly, such protocols may be used. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### III. ARRAY PREPARATION AND SCREENING

A. Array Preparation

Some embodiments involve the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, and silicon. Such arrays may vary in a number of different ways, including average probe length, sequence or types of probes, nature of bond between the probe and the array surface, e.g. covalent or non-covalent, and the like. The labeling and screening methods are not limited by with respect to any parameter except that the probes detect miRNA; consequently, methods and compositions may be used with a variety of different types of miRNA arrays.

Representative methods and apparatuses for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000, which are each herein incorporated by reference.

It is contemplated that the arrays can be high density arrays, such that they contain 2, 20, 25, 50, 80, 100, or more, or any integer derivable therein, different probes. It is contemplated that they may contain 1000, 16,000, 65,000, 250,000 or 1,000,000 or more, or any interger or range derivable therein, different probes. The probes can be directed to targets in one or more different organisms or cell types. In some embodiments, the oligonucleotide probes may range from 5 to 50, 5 to 45, 10 to 40, 9 to 34, or 15 to 40 nucleotides in length. In certain embodiments, the oligonucleotide probes are 5, 10, 15, 20, 25, 30, 35, 40 nucleotides in length, including all integers and ranges there between.

Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed herein or may be found in, for example, WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the miRNA of a wide variety of samples can be analyzed using arrays, miRNA probes, or array technology. While endogenous miRNA is contemplated for use with compositions and methods disclosed herein, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells. In certain embodiments, samples may be, but are not limited to, fresh, frozen, fixed, formalin fixed, paraffin embedded, or formalin fixed and paraffin embedded. Alternatively, the sample may not be a biological sample, but a chemical mixture, such as a cell-free reaction mixture (which may contain one or more biological enzymes).

1. Biological Sample Collection

In certain aspects, methods involve obtaining a sample from a subject. The term subject may refer to an animal (for example a mammal), including but not limited to humans, non-human primates, rodents, dogs, or pigs. The methods of obtaining provided herein include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from pancreas or pancreatic tissue by any of the biopsy methods previously mentioned. In other embodiments the sample may be obtained from any of the tissues provided herein that include but are not limited to gall bladder, skin, heart, lung, breast, pancreas, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In yet other embodiments the cyst, tumor or neoplasm is pancreatic. In certain aspects of the current methods, any medical professional such as a doctor, nurse or medical technician may obtain a biological sample for testing.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples, such as multiple pancreatic samples may be obtained for diagnosis by the methods described herein. In other cases, multiple samples, such as one or more samples from one tissue type (for example pancreas) and one or more samples from another tissue (for example buccal) may be obtained for diagnosis by the methods of the present methods. In some cases, multiple samples such as one or more samples from one tissue type (e.g. pancreas) and one or more samples from another tissue (e.g. buccal) may be obtained at the same or different times. Samples may be obtained at different times are stored and/or analyzed by different methods. For example, a sample may be obtained and analyzed by routine staining methods or any other cytological analysis methods.

In some cases, further samples may be obtained from a subject based on the results of such a cytological analysis. A cancer diagnosis may include an examination of a subject by a physician, nurse or other medical professional. The examination may be part of a routine examination, or the examination may be due to a specific complaint. A specific complaint may include but is not limited to: pain, illness, anticipation of illness, presence of a suspicious lump or mass, a disease, or a condition.

In some embodiments the subject may or may not be aware of the disease or condition.

In some cases, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a pancreatic cyst or a suspected pancreatic tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

2. Biological Sample Storage

In certain aspects, a sample may be obtained and prior to analysis by one or more methods described herein, the sample may be stored for a length of time. A length of time may include a time interval such as seconds, minutes, hours, days, weeks, months, years or longer. In some cases, the sample obtained from a subject is subdivided prior to the step of storage or further analysis. In some cases where the cample is subdivided different portions of the sample are subjected to different downstream methods or processes. Such methods or processes may include storage, cytological analysis, integrity tests, nucleic acid extraction, molecular profiling or any combination of these.

In some cases where storage is contemplated, some part of the sample may be stored while another portion of the sample is further processed. Processing may include but is not limited to molecular profiling, cytological staining, gene or gene expression product (RNA or protein) extraction, detection, or quantification, fixation or examination.

In other cases, the sample is obtained and stored and subdivided after the step of storage for further analysis such that different portions of the sample are subject to different downstream methods or processes including but not limited to storage, cytological analysis, adequacy tests, nucleic acid extraction, molecular profiling or a combination thereof.

In some cases, samples are obtained and analyzed by cytological analysis, and the resulting sample material is further analyzed by one or more molecular profiling methods described herein. In such cases, the samples may be stored between the steps of cytological analysis and the steps of molecular profiling. Samples may be stored upon acquisition to facilitate transport, or to wait for the results of other analyses. In another embodiments, samples may be stored while awaiting instructions a medical professional.

An acquired sample may be placed in short term or long term storage by placing in a suitable medium, excipient, solution, or container. In certain cases storage may require keeping the sample in a refrigerated, or frozen environment. The sample may be quickly frozen prior to storage in a frozen environment. In certain instances the frozen sample may be contacted with a suitable cryopreservation medium or compound. Examples of cryopreservation mediums or compounds include but are not limited to: glycerol, ethylene glycol, sucrose, or glucose.

A suitable medium, excipient, or solution may include but is not limited to: hanks salt solution, saline, cellular growth medium, an ammonium salt solution such as ammonium sulphate or ammonium phosphate, or water.

Suitable concentrations of ammonium salts include solutions of about 0.1 g/ml, 0.2 g/ml, 0.3 g/ml, 0.4 g/ml, 0.5 g/ml, 0.6 g/ml, 0.7 g/ml, 0.8 g/ml, 0.9 g/ml, 1.0 g/ml, 1.1 g/ml, 1.2 g/ml, 1.3 g/ml, 1.4 g/ml, 1.5 g/ml, 1.6 g/ml, 1.7 g/ml, 1.8 g/ml, 1.9 g/ml, 2.0 g/ml, 2.2 g/ml, 2.3 g/ml, 2.5 g/ml, 2.7 g/ml, 3.0 g/ml or higher. The medium, excipient, or solution may or may not be sterile.

The medium, excipient, or solution may contain preservative agents to maintain the sample in an adequate state for subsequent diagnostics or manipulation, or to prevent coagulation. Said preservatives may include citrate, ethylene diamine tetraacetic acid, sodium azide, or thimersol. The sample may be fixed prior to or during storage by any method known to the art such as using glutaraldehyde, formaldehyde, or methanol. The container may be any container suitable for storage and or transport of the biological sample including but not limited to: a cup, a cup with a lid, a tube, a sterile tube, a vacuum tube, a syringe, a bottle, a microscope slide, or any other suitable container. The container may or may not be sterile. In some cases, the sample may be stored in a commercial preparation suitable for storage of cells for subsequent cytological analysis such as but not limited to Cytyc ThinPrep, SurePath, or Monoprep.

The storage temperature may be explicitly defined or defined by a temperature range. The sample may be stored at room temperature or at reduced temperatures such as cold temperatures (e.g. between about 20° C. and about 0° C.), or freezing temperatures, including for example 0° C., −1° C., −2° C., −3° C., −4° C., −5° C., −6° C., −7° C., −8° C., −9° C., −10° C., −12° C., −14° C., −15° C., −16° C., −20° C., −22° C., −25° C., −28° C., −30° C., −35° C., −40° C., −45° C., −50° C., −60° C., −70° C., −80° C., −100° C., −120° C., −140° C., −180° C., −190° C., or about −200° C. The sample may be stored in any condition or environment that allows or achieves the desired temperature condition. In some cases, the samples may be stored in a refrigerator, on ice or a frozen gel pack, in a freezer, in a cryogenic freezer, on dry ice, in liquid nitrogen, or in a vapor phase equilibrated with liquid nitrogen.

The sample container may be any container suitable for storage and or transport of the biological sample including but not limited to: a cup, a cup with a lid, a tube, a sterile tube, a vacuum tube, a syringe, a bottle, a microscope slide, or any other suitable container. The container may or may not be sterile.

3. Sample Conveyance and Transportation

Additionally contemplated in the current methods are methods of transporting a sample. Transport may involve moving or conveyance of a sample to or from a clinic, hospital, doctor's office, or other location to a second location. Upon transport the sample may be stored and/or analyzed by for example, cytological analysis or molecular profiling. In some embodiments some aspect of analysis, processing or profiling may begin or take place during transport. In some cases, the sample may be transported to a molecular profiling company in order to perform the analyses described herein. In other cases, the sample may be transported to a laboratory such as a laboratory authorized or otherwise capable of performing the methods described herein, such as a Clinical Laboratory Improvement Amendments (CLIA) laboratory.

In some instances the subject may transport the sample. Transportation by an individual may include the individual appearing at a molecular profiling business or a designated sample receiving point and providing a sample. Providing of the sample may involve any of the techniques of sample acquisition described herein, or the sample may have already have been acquired and stored in a suitable container. In other cases the sample may be transported to a molecular profiling business using a courier service, the postal service, a shipping service, or any method capable of transporting the sample in a suitable manner.

In some cases, the sample may be provided to a molecular profiling business by a third party testing laboratory (e.g. a cytology lab). In other cases, the sample may be provided to a molecular profiling business by the subject's primary care physician, endocrinologist or other medical professional. The cost of transport may be billed to the individual, medical provider, or insurance provider. The molecular profiling business may begin analysis of the sample immediately upon receipt, or may store the sample in any manner described herein. The method of storage may or may not be the same as chosen prior to receipt of the sample by the molecular profiling business.

The sample may be transported in any medium or excipient including any medium or excipient provided herein suitable for storing the sample such as a cryopreservation medium or a liquid based cytology preparation. In some cases, the sample may be transported frozen or refrigerated such as at any of the suitable sample storage temperatures provided herein.

Once the sample is received, the sample may be assayed using a variety of routine analyses known to the art such as cytological assays, and genomic analysis by a molecular profiling business, a representative or licensee thereof, a medical professional, researcher, or a third party laboratory or testing center (e.g. a cytology laboratory). Such tests may be indicative of cancer, the type of cancer, any other disease or condition, the presence of disease markers, or the absence of cancer, diseases, conditions, or disease markers. The tests may take the form of cytological examination including microscopic examination as described below. The tests may involve the use of one or more cytological stains. The biological material may be manipulated or prepared for the test prior to administration of the test by any suitable method known to the art for biological sample preparation. The specific assay performed may be determined by the molecular profiling company, the physician who ordered the test, or a third party such as a consulting medical professional, cytology laboratory, the subject from whom the sample derives, or an insurance provider. The specific assay may be chosen based on the likelihood of obtaining a definite diagnosis, the cost of the assay, the speed of the assay, or the suitability of the assay to the type of material provided.

4. Sample Integrity Tests

In some embodiments, concurrent with sample aquistion, sample storage or sample analysis the sample may be subjected to tests or examination that detail or reveal the integrity of the sample for use in the compositions or methods described herein. As a result of an integrity test a sample may be determined to be adequate or inadequate for further analysis.

In some embodiments sample integrity tests may pertain to the quality, integrity or adequacy of cells and or tissue in the sample. Metrics employed to determine quality, integrity or adequacy may involve but are not limited to cell number tests, cell viability tests, nuclear content tests, genetic content tests, biochemical assays, cell mass tests, cell volume tests, PCR tests, Q-PCR tests, RT-PCR tests, immunochemical analysis, histochemical analysis, microscopic analysis or visual analysis.

In certain aspects sample integrity may be ascertained by tests that measure nucleic acid content or integrity. Nucleic acid content tests may measure DNA content, RNA content or a some mixture of DNA or RNA. In some aspects nucleic acids are extracted or purified from other cellular components prior to a nucleic acid content test. In some embodiments nucleic acid specific dyes are used to assay nucleic acid integrity. In cases of nucleic acid extraction, spectrophotometric or electrophoretic methods may be used to assay nucleic acid integrity.

In yet other aspects, sample integrity may be ascertained by tests that measure protein content or integrity. Methods that measure protein content or integrity are well known to those skilled in the art. Such methods include but are not limited to ultraviolet absorbance reading (e.g. 280 nm absorbance readings), cell staining, protein staining or immunocytochemical methods. In some instances tests may be performed in parallel in intact samples or the samples may be divided and tests performed serially or in parallel.

Integrity tests may be performed on small subsets or aliquots of a sample or on the entirety of a sample.

C. Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays can be used to detect differences between two samples. Specifically contemplated applications include identifying and/or quantifying differences between miRNA from a sample that is normal and from a sample that is not normal, between a cancerous condition and a non-cancerous condition, or between two differently treated samples. Also, miRNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. No. 5,384,261, incorporated herein by reference in its entirety. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate (see U.S. Pat. Nos. 5,770,358, 5,789,162, 5,708,153, 6,040,193 and 5,800,992, each of which is hereby incorporated in its entirety). Arrays may be packaged in such a manner as to allow for diagnostics or other manipulation of an all inclusive device (see for example, U.S. Pat. Nos. 5,856,174 and 5,922,591, each incorporated in its entirety by reference). See also U.S. patent application Ser. No. 09/545,207, filed Apr. 7, 2000, which is incorporated by reference in its entirety for additional information concerning arrays, their manufacture, and their characteristics,

Particularly, arrays can be used to evaluate samples with respect to diseases or conditions that include, but are not limited to: chronic pancreatitis; acute pancreatitis, autoimmune pancreatitis; pancreatic cancer; AIDS, autoimmune diseases (rheumatoid arthritis, multiple sclerosis, diabetes—insulin-dependent and non-independent, systemic lupus erythematosus and Graves disease); cancer (e.g., malignant, benign, metastatic, precancer); cardiovascular diseases (heart disease or coronary artery disease, stroke—ischemic and hemorrhagic, and rheumatic heart disease); diseases of the nervous system; and infection by pathogenic microorganisms (Athlete's Foot, Chickenpox, Common cold, Diarrheal diseases, Flu, Genital herpes, Malaria, Meningitis, Pneumonia, Sinusitis, Skin diseases, Strep throat, Tuberculosis, Urinary tract infections, Vaginal infections, Viral hepatitis); inflammation (allergy, asthma); prion diseases (e.g., CJD, kuru, GSS, FFI).

Moreover, miRNAs can be evaluated with respect to the following diseases, conditions, and disorders: pancreatitis, chronic pancreatitis, and/or pancreatic cancer. In addition, miRNAs can be evaluated with respect to pancreatic cysts and the determination of whether a particular cyst is not generally malignant or aggressive (e.g., determining whether the cyst is aserous cystadenoma (SCA), low grade intraductal papillary mucinous neoplasm (LG-IPMN)). Also, miRNAs can be evaluated with respect to determining whether a particular cyst is generally considered malignant and/or aggressive, and this includes, but is not limited to mucinous cystic neoplasm (MCN), solid pseudopapillary neoplasm (SPN), neuroendocrine tumor (NET), and high grade intraductal papillary mucinous neoplasm (HG-IPMN). Moreover, miRNAs can be evaluated in precancers, such as metaplasia, dysplasia, and hyperplasia.

It is specifically contemplated that the disclosed methods and compositions can be used to evaluate differences between stages of disease, such as between hyperplasia, neoplasia, pre-cancer and cancer, or between a primary tumor and a metastasized tumor, or between a lesion that is low risk (i.e., not generally malignant or aggressive) and a lesion that is high risk (i.e., generally considered malignant and/or aggressive).

In embodiments described herein, differential expression was observed in different pancreatic cyst populations. These include, but are not limited to those in the following (based on high risk lesions as compared to low risk lesions generally):

| miR | Nominal Direction (based on raw Cts) | |
|---|---|---|
| miR-24 | Up | Used as normalizer in some other studies |
| miR-30a-3p | Flat | Perhaps up slightly for all groups other than SCA |
| miR-18a | Up | |
| miR-92a | Up | Up mainly for NET and SPN groups, less for HG IPMNs |
| miR-342-3p | Up | |
| miR-99b | Up | Up mainly for NET and SPN groups, less for HG IPMNs |
| miR-106b | Up | |
| miR-142-3p | Up | |
| miR-532-3p | Up | Up mainly for NET and SPN groups, less for HG IPMNs |

E. Other Assays

In addition to the use of arrays and microarrays, it is contemplated that a number of different assays could be employed to analyze miRNAs, their activities, and their effects. Such assays include, but are not limited to, nucleic acid amplification, polymerase chain reaction, quantitative PCR, RT-PCR, in situ hybridization, Northern hybridization, hybridization protection assay (HPA), branched DNA (bDNA) assay, rolling circle amplification (RCA), single molecule hybridization detection, Invader assay, and/or Bridge Litigation Assay.

F. Evaluation of Expression Levels and Diff Pair Values

A variety of different models can be employed to evaluate expression levels and/or other comparative values based on expression levels of miRNAs (or their precursors or targets). One model used in the Examples described below is a logistic regression model (see the Wikipedia entry on the World Wide Web at en.wikipedia., which is hereby incorporated by reference).

Start by computing the weighted sum of the DiffPair values:

z=β₀β₁*Diff(miR₁ₐ,miR_{1b})+β₂*Diff(miR₂ₐ,miR_{2b})⁺ . . .

where the β₀ is the (Intercept) term identified in the spreadsheets, while the remaining βᵢ are the weights corresponding to the various DiffPairs in the model in question. Once z is computed, the score p_{malignant} (which may be interpreted as predicted probability of malignancy) is calculated as

$${p}_{malignant} = \frac{1}{1 + exp ( - z )}$$

This functions to turn the number z, which may be any value from negative infinity to positive infinity, into a number between 0 and 1, with negative values for z becoming scores/probabilities of less than 50% and positive values for z becoming scores/probabilities of greater than 50%.

Other examples of models include but are not limited to Decision Tree, Linear Disciminant Analysis, Neural Network, Support Vector Machine, and k-Nearest Neighbor Classifier. A person of ordinary skill in the art could use these different models to evaluate expression level data and comparative data involving expression levels of one or more miR5 (or their precursors or their targets).

Models may take into account one or more diff pair values or they may also take into account differential expression of one or more miRNAs not specifically as part of a diff pair. A diagnostic score may be based on 1, 2, 3, 4, 5, 6, 7, 8 or more diff pair values (or any range derivable therein), but in some embodiments, it takes into account additionally or alternatively, 1, 2, 3, 4, 5, 6, 7, 8, 9, 10 or more miRNA expression levels (or any range derivable therein), wherein the miRNA expression level detectably differs between low risk and high risk lesions.

It will be understood by those of skill in the art that instead of a diff pair value, methods may involve a coefficient value that can be used in conjunction with the level of expression of a particular miRNA based on classifier analysis. Whereas the expression value associated with a diff pair is 1 times the expression value of the first miRNA of the pair summed with −1 times the expression value of the second miRNA of the diff pair, a more general classifier may be built with features composed of combinations of 2 or more miRNA expression values with coefficient values other than just 1 or −1.

Additionally, embodiments may be based on a constrained logistic regression model. For illustrative purposes, consider a model built with three biomarker predictors A, B, and C. The classifier is specified by the weights w₀, w₁, w₂, and w₃: given a sample with expression value x₁ for marker A, x₂ for marker B, and x₃ for marker C, the model score may be computed as

$${p}_{malignant} = \frac{1}{1 + exp ( - z )} = \frac{1}{1 + exp ( - {w}_{0} - {w}_{1} {x}_{1} - {w}_{2} {x}_{2} - {w}_{3} {x}_{3} )}$$

$$where$$

$$\begin{matrix}z = {w}_{0} + {w}_{1} {x}_{1} + {w}_{2} {x}_{2} + {w}_{3} {x}_{3} \\ = {w}_{0} + {w}_{1} ( {x}_{1} - {x}_{2} ) + ( {w}_{1} + {w}_{2} ) ( {x}_{2} - {x}_{3} ) + ( {w}_{1} + {w}_{2} + {w}_{3} ) {x}_{3}\end{matrix}$$

Since a model may be constrained to satisfy w1+w2+w3=0, the last term in the above equation must vanish. But what's left expresses our model score in terms of the diff pairs Diff(A,B) (with expression value x1−x2) and Diff(B,C) (with expression value x2−x3). This argument can be extended in a straight-forward manner to apply to constrained logistic regression models with any number of predictors.

Alternatively, the converse may be done. A logistic regression model built using two DiffPairs, Diff(A,B) and Diff(C,D), with weights W₁ and W₂ can be equivalently described directly in terms of the underlying miRs A, B, C, and D with weights w₁=W₁, w₂=−W₁, w₃=W₂, and W₄=−W₂ (in case of miR overlap, we just add together the relevant coefficients: e.g., if B=C, we would have w₁=W₁, w₂=−W₁+W₂, w₃=−W₂, with x₃ now describing the expression value of D). Because the weight of each diff pair appears twice in the resulting individual miRs, once with a positive sign and once with a negative sign, the sum of the resulting individual miR coefficients (the lower-case wᵢ's) must be zero, satisfying our constraint condition.

### IV. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well as reagents for preparation of samples from pancreatic samples. The kit may further include reagents for creating or synthesizing miRNA probes. Such kits may thus comprise, in suitable container means, an enzyme for labeling the miRNA by incorporating labeled nucleotides or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. Kits may also include one or more buffers, such as a reaction buffer, labeling buffer, washing buffer, or hybridization buffer, compounds for preparing the miRNA probes, and components for isolating miRNAs. Other kits may include components for making a nucleic acid array comprising miRNAs, and thus, may include, for example, a solid support.

Kits for implementing methods described herein are specifically contemplated. In some embodiments, there are kits for preparing miRNAs for multi-labeling and kits for preparing miRNA probes and/or miRNA arrays. In such embodiments, kits comprise, in suitable container means, 1, 2, 3, 4, 5, 6, 7, 8, 9, 10, 11, 12 or more of the following: 1) poly(A) polymerase; 2) unmodified nucleotides (G, A, T, C, and/or U); 3) a modified nucleotide (labeled or unlabeled); 4) poly(A) polymerase buffer; 5) at least one microfilter; 6) label that can be attached to a nucleotide; 7) at least one miRNA probe; 8) reaction buffer; 9) a miRNA array or components for making such an array; 10) acetic acid; 11) alcohol; 12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits include an array containing miRNA probes, as described in the application. An array may have probes corresponding to all known miRNAs of an organism or a particular tissue or organ in particular conditions, or to a subset of such probes. The subset of probes on arrays may be or include those identified as relevant to a particular diagnostic, therapeutic, or prognostic application. For example, the array may contain one or more probes that are indicative or suggestive of 1) a disease or condition (chronic pancreatitis and/or pancreatic cancer), 2) susceptibility or resistance to a particular drug or treatment; 3) susceptibility to toxicity from a drug or substance; 4) the stage of development or severity of a disease or condition (prognosis); and 5) genetic predisposition to a disease or condition.

For any kit embodiment, including an array, there can be nucleic acid molecules that contain or can be used to amplify a sequence that is a variant of, identical to, or complementary to all or part of any of the sequences disclosed herein. In certain embodiments, a kit or array can contain one or more probes for the miRNAs identified by sequences disclosed herein. Any nucleic acid discussed above may be implemented as part of a kit.

Components of kits may be packaged either in aqueous media or in lyophilized form. The container means of the kits will generally include at least one vial, test tube, flask, bottle, syringe, or other container means, into which a component may be placed, and preferably, suitably aliquotted. Where there is more than one component in the kit (e.g., labeling reagent and label may be packaged together), the kit also will generally contain a second, third, or other additional container into which the additional components may be separately placed. However, various combinations of components may be comprised in a vial. The kits also may include a means for containing the nucleic acids, and any other reagent containers in close confinement for commercial sale. Such containers may include injection or blow molded plastic containers into which the desired vials are retained.

When the components of a kit are provided in one and/or more liquid solutions, the liquid solution may be an aqueous solution, with a sterile aqueous solution being particularly preferred.

However, the components of a kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg, or at least or at most those amounts, of dried dye are provided in kits. The dye may then be resuspended in any suitable solvent, such as DMSO.

The container means will generally include at least one vial, test tube, flask, bottle, syringe and/or other container means, into which the nucleic acid formulations are placed, for example, suitably allocated. Kits may also comprise a second container means for containing a sterile, pharmaceutically acceptable buffer and/or other diluent.

Kits may include a means for containing the vials in close confinement for commercial sale, such as, e.g., injection and/or blow-molded plastic containers into which the desired vials are retained.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit may also include instructions for employing the kit components as well the use of any other reagent not included in the kit. Instructions may include variations that can be implemented.

Kits may also include one or more of the following: control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits. Such kits, however, are not limited to the particular items identified above and may include any reagent used for the manipulation or characterization of miRNA.

### V. EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art will, in light of the present disclosure, appreciate that many changes can be made in the specific embodiments that are disclosed and still obtain a like or similar result without departing from the spirit and scope of the invention.

### Example 1

The diagnostic benefit of using miRNAs as biomarkers in pancreatic cyst fluid was assessed, focusing on IPMNs due to their frequency and malignant potential. In brief, RNA was extracted from 61 microdissected FFPE IPMNs and 65 cyst fluid specimens. Expression profiling of 750 miRNAs was performed using TaqMan MicroRNA Arrays using FFPE (n=23) and cyst fluid (n=15) specimens. Differential expression was verified in FFPE (n=38) and cyst fluid (n=50) specimens using TaqMan MicroRNA Assays.

The study yielded miRNAs that identified patients with HG IPMN and ruled out non-mucinous cysts. Specifically, the study identified 26 and 37 miRNAs that are differentially expressed in LG and HG IPMNs, respectively, with 4 miRNAs in common (let-7c, miR-106b, -342-3p and -93). 19 miRNAs selected using both FFPE and cyst fluid data separated HG from not only LG IPMNs and serous cystadenomas (SCAs), but also from uncommon cysts such as solid pseudopapillary neoplasms (SPNs) and cystic neuroendocrine tumors (NETs). Logistic regression allowed prediction of cyst pathology implying resection (HG IPMNs, NETs, SPNs) vs. conservative management (LG IPMNs, SCAs) with an accuracy of 90% and 100%, respectively.

### Methods

Patients and Biospecimens.

This study was approved by the Johns Hopkins University Institutional Review Board. The prospectively maintained Johns Hopkins Surgical Pathology Database was used to identify formalin-fixed, paraffin-embedded (FFPE) tissue specimens of patients who underwent pancreatectomy for IPMN between Jan. 1, 2000, and Aug. 31, 2010, at Johns Hopkins hospital. Hematoxylin and eosin stained (H+E) reference slides were used to identify samples for subsequent molecular studies.

Histologic diagnoses were reconfirmed by two pathologists according to the latest World Health Organization recommendations (WHO) (Bosman et al., 2010). A consensus was reached in all cases. Briefly, IPMNs had to display a papillary epithelium with abundant extracellular mucin and measure per definition >1 cm in maximum diameter. Main duct IPMNs were distinguished from branch duct IPMNs, and a third category of a mixed type was assigned whenever the lesion was located in both main and branch duct. In each case, the final diagnosis referred to the most severe grade of dysplasia observed in the neoplastic epithelium, including LG, intermediate grade (IG), and HG IPMNs. Furthermore, the inventors assessed whether an IPMN had an associated invasive carcinoma and sub-classified invasive lesions into colloid and ductal adenocarcinoma (Matthaei and Maitra, 2011).

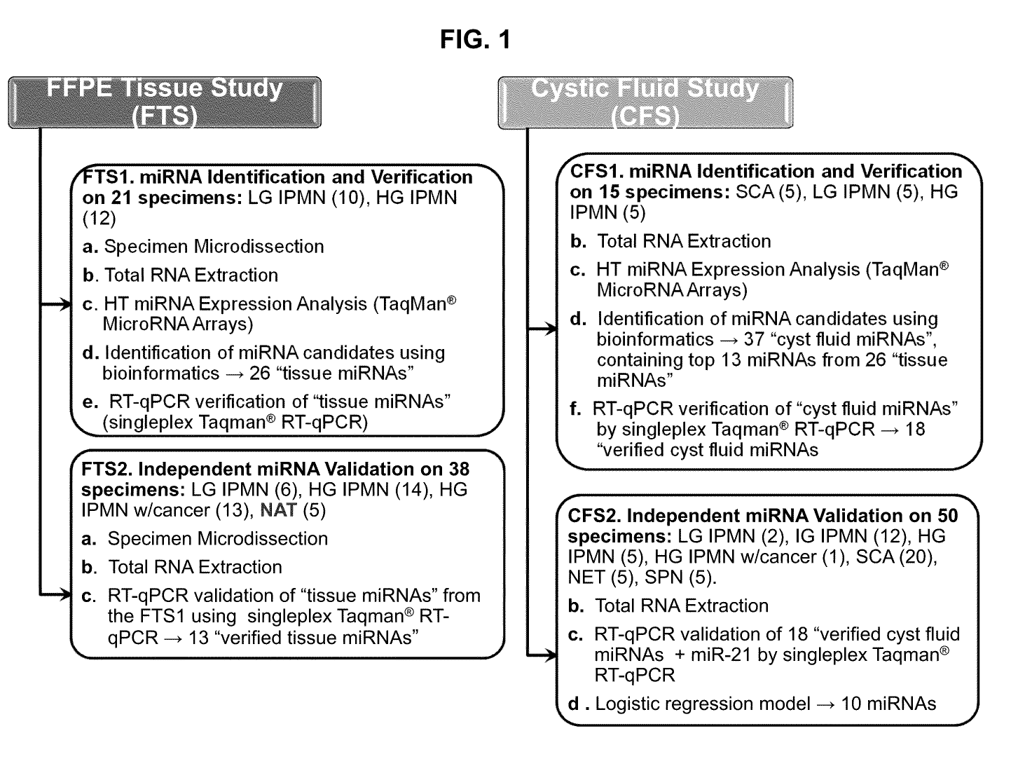

For unbiased high-throughput (HT) miRNA expression profiling (“FFPE tissue study 1” or “FTS1”, FIG. 1) 22 IPMNs, including 10 LG IPMNs and 12 HG IPMNs, were selected. Seven of the latter had an associated invasive adenocarcinoma. For validation of candidate miRNAs in an independent set of specimens (“FFPE tissue study 2” or “FTS2”, FIG. 1), an additional 33 archival IPMNs were selected (6 LG IPMNs, 14 HG IPMNs and 13 HG IPMNs with an associated invasive adenocarcinoma). Five normal adjacent specimens were harvested from specimens Valid-FT15, -FT19, -FT24, -FT27 and -FT30 to serve as references. All lesions included are listed in Table 1, which provides total RNA recovery, demographic and tumor-related information for the FFPE tissue specimens. Summary statistics for LG and HG IPMNs are included, and specimens excluded from bioinformatics analyses are indicated.

TABLE 1
FFPE Tissue Samples

| | | Concentration | total RNA | | Bioinformatics Inclusion |
|---|---|---|---|---|---|
| Study | Specimen ID | (ng/μl) | yield (ng) | Experimental grouping | Status |
| FTS1 | Disc-FT1 | 10.50 | 262.5 | IPMN low-grade | Included |
| | Disc-FT10 | 17.2 | 430 | IPMN low-grade | Included |
| | Disc-FT11 | 47.90 | 1197.5 | IPMN high-grade | Included |
| | Disc-FT12 | 133.20 | 3330 | IPMN high-grade | Included |
| | Disc-FT13 | 79.80 | 1995 | IPMN high-grade(with | Included |
| | | | | associated invasive carcinoma) | |
| | Disc-FT14 | 35.90 | 897.5 | IPMN high-grade(with | Included |
| | | | | associated invasive carcinoma) | |
| | Disc-FT15 | 27.80 | 695 | IPMN high-grade | Included |
| | Disc-FT16 | 30.40 | 760 | IPMN high-grade | Included |
| | Disc-FT17 | 160.80 | 4020 | IPMN high-grade | Included |
| | Disc-FT18 | 59.20 | 1480 | IPMN high-grade(with | Included |
| | | | | associated invasive carcinoma) | |
| | Disc-FT19 | 36.00 | 900 | IPMN high-grade(with | Included |
| | | | | associated invasive carcinoma) | |
| | Disc-FT2 | 31.60 | 790 | IPMN low-grade | Included |
| | Disc-FT20 | 97.50 | 2437.5 | IPMN high-grade(with | Included |
| | | | | associated invasive carcinoma) | |
| | Disc-FT21 | 145.70 | 3642.5 | IPMN high-grade(with | Included |
| | | | | associated invasive carcinoma) | |
| | Disc-FT22 | 186.00 | 4650 | IPMN high-grade(with | Included |
| | | | | associated invasive carcinoma) | |
| | Disc-FT23 | 86.7 | 2167.5 | Normal pancreas | Included |
| | Disc-FT3 | 10.30 | 257.5 | IPMN low-grade | Included |
| | Disc-FT4 | 23.90 | 597.5 | IPMN low-grade | Included |
| | Disc-FT5 | 20.10 | 502.5 | IPMN low-grade | Included |
| | Disc-FT6 | 13.40 | 335 | IPMN low-grade | Included |
| | Disc-FT7 | 17.20 | 430 | IPMN low-grade | Included |
| | Disc-FT8 | 9.80 | 245 | IPMN low-grade | Included |
| | Disc-FT9 | 25.40 | 635 | IPMN low-grade | Included |
| FTS2 | Valid-FT1 | 58.3 | 1749 | IPMN high-grade | Included |
| | Valid-FT10 | 21.8 | 654 | IPMN high-grade(with | Included |
| | | | | associated invasive carcinoma) | |
| | Valid-FT11 | 18.6 | 558 | IPMN high-grade | Included |
| | Valid-FT12 | 32.2 | 966 | IPMN high-grade(with | Included |
| | | | | associated invasive carcinoma) | |
| | Valid-FT13 | 24 | 720 | IPMN high-grade | Included |
| | Valid-FT14 | 33.5 | 1005 | IPMN high-grade | Included |
| | Valid-FT15 | 18.1 | 543 | IPMN low-grade | Included |
| | Valid-FT16 | 33.7 | 1011 | IPMN high-grade(with | Included |
| | | | | associated invasive carcinoma) | |
| | Valid-FT17 | 19.6 | 588 | IPMN high-grade | Included |
| | Valid-FT18 | 14.6 | 438 | IPMN high-grade | Included |
| | Valid-FT19 | 8.3 | 249 | IPMN low-grade | Excluded: many missing values |
| | Valid-FT2 | 5.3 | 159 | IPMN high-grade | Excluded: many missing values |
| | Valid-FT20 | 8.3 | 249 | IPMN high-grade | Excluded: many missing values |
| | Valid-FT21 | 6.5 | 195 | IPMN high-grade | Excluded: many missing values |
| | Valid-FT22 | 7.7 | 231 | IPMN high-grade | Excluded: many missing values |
| | Valid-FT23 | 6.8 | 204 | IPMN high-grade(with | Included |
| | | | | associated invasive carcinoma) | |
| | Valid-FT24 | 19.4 | 582 | IPMN low-grade | Included |
| | Valid-FT25 | 55.7 | 1671 | IPMN high-grade | Included |
| | Valid-FT26 | 41.2 | 1236 | IPMN high-grade(with | Included |
| | | | | associated invasive carcinoma) | |
| | Valid-FT27 | 18.9 | 567 | IPMN low-grade | Included |
| | Valid-FT28 | 43.1 | 1293 | IPMN high-grade | Included |
| | Valid-FT29 | 44.9 | 1347 | IPMN high-grade(with | Included |
| | | | | associated invasive carcinoma) | |
| | Valid-FT3 | 12.2 | 366 | IPMN high-grade(with | Excluded: mean Ct > 30 |
| | | | | associated invasive carcinoma) | |
| | Valid-FT30 | 8.7 | 261 | IPMN low-grade | Excluded: many missing values |
| | Valid-FT31 | 14.4 | 432 | IPMN high-grade(with | Included |
| | | | | associated invasive carcinoma) | |
| | Valid-FT32 | 16.4 | 492 | IPMN high-grade(with | Included |
| | | | | associated invasive carcinoma) | |
| | Valid-FT33 | 8.3 | 249 | IPMN high-grade(with | Excluded: many missing values |
| | | | | associated invasive carcinoma) | |
| | Valid-FT34 | 69.5 | 2085 | normal pancreas of Valid-FT15 | Included |
| | Valid-FT35 | 16.3 | 489 | normal pancreas of Valid-FT19 | Included |
| | Valid-FT36 | 55 | 1650 | normal pancreas of Valid-FT24 | Included |
| | Valid-FT37 | 73.1 | 2193 | normal pancreas of Valid-FT27 | Included |
| | Valid-FT38 | 118.4 | 3552 | normal pancreas of Valid-FT30 | Included |
| | Valid-FT4 | 15.1 | 453 | IPMN high-grade | Included |
| | Valid-FT5 | 6.5 | 195 | IPMN high-grade | Excluded: many missing values |
| | Valid-FT6 | 14.8 | 444 | IPMN low-grade | Excluded: mean Ct > 30 |
| | Valid-FT7 | 45.9 | 1377 | IPMN high-grade(with | Included |
| | | | | associated invasive carcinoma) | |
| | Valid-FT8 | 37.3 | 1119 | IPMN high-grade(with | Included |
| | | | | associated invasive carcinoma) | |
| | Valid-FT9 | 12.2 | 366 | IPMN high-grade(with | Included |
| | | | | associated invasive carcinoma) | |

65 samples were selected from the prospectively maintained Johns Hopkins Cyst Fluid Bank, all of which were harvested from pancreatic cysts resected at the Department of Surgery, Johns Hopkins Hospital. All cyst fluids were aspirated immediately after surgical resection in the Department of Surgical Pathology using a sterile syringe, aliquoted in sterile 1.5 ml tubes and stored at −80° C. within 30 min after resection. Histologic diagnoses of the 15 CF specimens used in HT miRNA expression profiling (“Cyst fluid study 1” or “CFS1”, FIG. 1) were as follows: 5 LG IPMNs, 5 HG IPMNs (3 had an associated invasive cancer) and 5 SCAs. For miRNA candidate validation (“Cyst fluid study 2” or “CFS2”, FIG. 1) an independent set of 50 cyst lesions with associated CF specimens was used, which was composed of 2 LG IPMNs, 12 IG IPMNs, 6 HG IPMNs (one with an associated invasive carcinoma), 20 SCAs, 5 pancreatic NETs and 5 SPNs. The lesions from which cyst fluids were harvested are provided in Table 2. Table 2 provides the total RNA recovery, demographic and tumor-related information for cyst fluid specimens. In Table 2, the term “benign” means “low risk” and the term “malignant” means “high risk.” Summary statistics for experimental groupings are included, and specimens excluded from bioinformatics analyses are indicated.

TABLE 2
Cyst Fluid Specimens

| | | RNA | | | total | Original | | | | | |
|---|---|---|---|---|---|---|---|---|---|---|---|
| | | concen- | | | RNA | specimen | | Avg RNA | Bioinformatics | Training/Test | |
| | Specimen | tration | A260/ | A260/ | yield | volume, | Experimental | yield/50 ml | Inclusion | Group | Predicted |
| Study | ID | (ng/ml) | 280 | 230 | (ng) | ml | grouping | CF | Status | Assignment | Group |
| CFS1 | Disc- | 132.04 | 1.37 | 1.09 | 13204 | 0.5 | IPMN low grade | 1320.40 | Included | Training | Benign |
| | CF1 | | | | | | | | | | (2.29%) |
| | Disc- | 411.72 | 1.36 | 1.75 | 41172 | 0.4 | IPMN high | 5146.50 | Included | Training | Malignant |
| | CF10 | | | | | | grade (with | | | | (100.00%) |
| | | | | | | | associated | | | | |
| | | | | | | | invasive | | | | |
| | | | | | | | carcinoma) | | | | |
| | Disc- | 5.48 | 1.96 | 0.4 | 548 | 1.5 | serous | 18.27 | Excluded: | NA | NA |
| | CF11 | | | | | | cystadenoma | | insufficient | | |
| | | | | | | | | | material | | |
| | Disc- | 9.56 | 1.99 | 0.29 | 956 | 1.5 | serous | 31.87 | Excluded: | NA | NA |
| | CF12 | | | | | | cystadenoma | | many | | |
| | | | | | | | | | missing | | |
| | | | | | | | | | values | | |
| | Disc- | 13.04 | 2 | 0.24 | 1304 | 1.5 | serous | 43.47 | Included | Training | Benign |
| | CF13 | | | | | | cystadenoma | | | | (0.00%) |
| | Disc- | 7.24 | 2.02 | 0.43 | 724 | 1.5 | serous | 24.13 | Excluded | NA | NA |
| | CF14 | | | | | | cystadenoma | | from Logistic | | |
| | | | | | | | | | Regression | | |
| | | | | | | | | | Model: | | |
| | | | | | | | | | incomplete | | |
| | | | | | | | | | singleplex | | |
| | | | | | | | | | data | | |
| | Disc- | 9.88 | 2.03 | 0.32 | 988 | 1.5 | serous | 32.93 | Included | Training | Benign |
| | CF15 | | | | | | cystadenoma | | | | (0.02%) |
| | Disc- | 8.20 | 1.41 | 0.14 | 820 | 1.5 | IPMN low grade | 27.33 | Excluded | NA | NA |
| | CF2 | | | | | | | | from Logistic | | |
| | | | | | | | | | Regression | | |
| | | | | | | | | | Model: | | |
| | | | | | | | | | incomplete | | |
| | | | | | | | | | singleplex | | |
| | | | | | | | | | data | | |
| | Disc- | 13.84 | 1.41 | 0.28 | 1384 | 1.5 | IPMN low grade | 46.13 | Included | Training | Benign |
| | CF3 | | | | | | | | | | (0.97%) |
| | Disc- | 4.92 | 1.44 | 0.39 | 492 | 1.5 | IPMN low grade | 16.40 | Excluded | NA | NA |
| | CF4 | | | | | | | | from Logistic | | |
| | | | | | | | | | Regression | | |
| | | | | | | | | | Model: | | |
| | | | | | | | | | incomplete | | |
| | | | | | | | | | singleplex | | |
| | | | | | | | | | data | | |
| | Disc- | 4.12 | 1.46 | 0.16 | 412 | 1.5 | IPMN low grade | 13.73 | Included | Training | Benign |
| | CF5 | | | | | | | | | | (0.18%) |
| | Disc- | 227.12 | 1.03 | 1.72 | 22712 | 0.6 | IPMN high | 1892.67 | Included | Training | Malignant |
| | CF6 | | | | | | grade | | | | (99.35%) |
| | Disc- | 91.28 | 1.16 | 0.54 | 9128 | 1.5 | IPMN high | 304.27 | Included | Training | Malignant |
| | CF7 | | | | | | grade | | | | (99.94%) |
| | Disc- | 1513.20 | 1.29 | 2.07 | 151320 | 0.7 | IPMN high | 10808.57 | Included | Training | Malignant |
| | CF8 | | | | | | grade (with | | | | (98.88%) |
| | | | | | | | associated | | | | |
| | | | | | | | invasive | | | | |
| | | | | | | | carcinoma) | | | | |
| | Disc- | 15.68 | 1.33 | 0.39 | 1568 | 1.5 | IPMN high | 52.27 | Excluded: | NA | NA |
| | CF9 | | | | | | grade (with | | mis- | | |
| | | | | | | | associated | | clustering | | |
| | | | | | | | invasive | | | | |
| | | | | | | | carcinoma) | | | | |
| FTS2 | Valid- | 3.12 | 1.47 | 0.38 | 312 | 0.05 | IPMN low grade | 312.00 | Included | Test | Benign |
| | CF1 | | | | | | | | | | (0.32%) |
| | Valid- | 3.4 | 1.63 | 0.19 | 340 | 1.20 | IPMN | 14.17 | Included | Test | Benign |
| | CF10 | | | | | | intermediate | | | | (0.69%) |
| | | | | | | | grade | | | | |
| | Valid- | 145.62 | 1.64 | 1.84 | 14562 | 0.50 | IPMN | 1456.20 | Included | Test | Benign |
| | CF11 | | | | | | intermediate | | | | (0.11%) |
| | | | | | | | grade | | | | |
| | Valid- | 30.62 | 1.64 | 1.23 | 3062 | 0.50 | IPMN | 306.20 | Included | Test | Malignant |
| | CF12 | | | | | | intermediate | | | | (99.95%) |
| | | | | | | | grade | | | | |
| | Valid- | 7.78 | 1.67 | 0.49 | 778 | 0.50 | IPMN | 77.80 | Included | Test | Benign |
| | CF13 | | | | | | intermediate | | | | (9.75%) |
| | | | | | | | grade | | | | |
| | Valid- | 6.71 | 1.69 | 0.77 | 671 | 0.50 | IPMN | 67.10 | Included | Test | Malignant |
| | CF14 | | | | | | intermediate | | | | (86.06%) |
| | | | | | | | grade | | | | |
| | Valid- | 1036.87 | 1.69 | 2.04 | 103687 | 0.35 | IPMN high | 14812.43 | Included | Training | Malignant |
| | CF15 | | | | | | grade | | | | (97.07%) |
| | Valid- | 156.29 | 1.78 | 1.94 | 15629 | 0.05 | IPMN high | 15629.00 | Included | Training | Malignant |
| | CF16 | | | | | | grade | | | | (99.07%) |
| | Valid- | 11.44 | 1.81 | 0.61 | 1144 | 0.20 | IPMN high | 286.00 | Included | Training | Malignant |
| | CF17 | | | | | | grade | | | | (95.26%) |
| | Valid- | 29.87 | 1.81 | 0.88 | 2987 | 0.40 | IPMN high | 373.38 | Included | Test | Malignant |
| | CF18 | | | | | | grade | | | | (90.39%) |
| | Valid- | 41.01 | 1.81 | 0.54 | 4101 | 0.30 | IPMN high | 683.50 | Included | Test | Benign* |
| | CF19 | | | | | | grade | | | | (42.02%) |
| | Valid- | 18.84 | 1.47 | 1.02 | 1884 | 0.30 | IPMN low grade | 314.00 | Included | Test | Benign |
| | CF2 | | | | | | | | | | (0.08%) |
| | Valid- | 254.94 | 1.84 | 1.75 | 25494 | 0.10 | IPMN high | 12747.00 | Included | Test | Malignant |
| | CF20 | | | | | | grade + cancer | | | | (67.24%) |
| | Valid- | 8.26 | 2.03 | 0.51 | 826 | 0.50 | Serous | 82.60 | Included | Training | Benign |
| | CF21 | | | | | | cystadenoma | | | | (5.53%) |
| | Valid- | 3.36 | 2.04 | 0.19 | 336 | 0.40 | Serous | 42.00 | Included | Training | Benign |
| | CF22 | | | | | | cystadenoma | | | | (0.00%) |
| | Valid- | 6.65 | 2.04 | 0.39 | 665 | 0.25 | Serous | 133.00 | Included | Training | Benign |
| | CF23 | | | | | | cystadenoma | | | | (0.01%) |
| | Valid- | 18.97 | 2.04 | 0.87 | 1897 | 0.40 | Serous | 237.13 | Included | Training | Benign |
| | CF24 | | | | | | cystadenoma | | | | (2.86%) |
| | Valid- | 4.33 | 2.05 | 0.27 | 433 | 1.20 | Serous | 18.04 | Included | Training | Benign |
| | CF25 | | | | | | cystadenoma | | | | (0.61%) |
| | Valid- | 2.43 | 2.07 | 0.22 | 243 | 1.40 | Serous | 8.68 | Included | Test | Benign |
| | CF26 | | | | | | cystadenoma | | | | (0.00%) |
| | Valid- | 21.48 | 2.08 | 0.83 | 2148 | 1.00 | Serous | 107.40 | Included | Test | Benign |
| | CF27 | | | | | | cystadenoma | | | | (0.00%) |
| | Valid- | 4.3 | 2.08 | 0.21 | 430 | 1.20 | Serous | 17.92 | Included | Test | Benign |
| | CF28 | | | | | | cystadenoma | | | | (0.00%) |
| | Valid- | 13.81 | 2.08 | 0.34 | 1381 | 1.00 | Serous | 69.05 | Included | Test | Benign |
| | CF29 | | | | | | cystadenoma | | | | (0.56%) |
| | Valid- | 3.87 | 1.47 | 0.56 | 387 | 0.05 | IPMN | 387.00 | Included | Test | Benign |
| | CF3 | | | | | | intermediate | | | | (4.53%) |
| | | | | | | | grade | | | | |
| | Valid- | 22.33 | 2.09 | 0.84 | 2233 | 0.50 | Serous | 223.30 | Included | Test | Benign |
| | CF30 | | | | | | cystadenoma | | | | (0.69%) |
| | Valid- | 7.92 | 2.09 | 0.11 | 792 | 1.20 | Serous | 33.00 | Included | Test | Benign |
| | CF31 | | | | | | cystadenoma | | | | (0.00%) |
| | Valid- | 9.39 | 2.09 | 0.24 | 939 | 0.35 | Serous | 134.14 | Included | Test | Benign |
| | CF32 | | | | | | cystadenoma | | | | (0.15%) |
| | Valid- | 6.74 | 2.11 | 0.48 | 674 | 0.15 | Serous | 224.67 | Included | Test | Benign |
| | CF33 | | | | | | cystadenoma | | | | (0.01%) |
| | Valid- | 4.02 | 2.11 | 0.17 | 402 | 0.10 | Serous | 201.00 | Included | Test | Benign |
| | CF34 | | | | | | cystadenoma | | | | (0.09%) |
| | Valid- | 5.17 | 2.11 | 0.16 | 517 | 1.40 | Serous | 18.46 | Included | Test | Benign |
| | CF35 | | | | | | cystadenoma | | | | (0.00%) |
| | Valid- | 18.66 | 2.11 | 0.85 | 1866 | 0.60 | Serous | 155.50 | Included | Test | Benign |
| | CF36 | | | | | | cystadenoma | | | | (3.69%) |
| | Valid- | 9.48 | 2.12 | 0.86 | 948 | 0.10 | Serous | 474.00 | Included | Test | Benign |
| | CF37 | | | | | | cystadenoma | | | | (0.00%) |
| | Valid- | 7.69 | 2.12 | 0.36 | 769 | 1.00 | Serous | 38.45 | Included | Test | Benign |
| | CF38 | | | | | | cystadenoma | | | | (0.01%) |
| | Valid- | 3.48 | 2.16 | 0.19 | 348 | 1.00 | Serous | 17.40 | Included | Test | Benign |
| | CF39 | | | | | | cystadenoma | | | | (0.00%) |
| | Valid- | 3.25 | 1.48 | 0.16 | 325 | 1.40 | IPMN | 11.61 | Included | Test | Benign |
| | CF4 | | | | | | intermediate | | | | (0.06%) |
| | | | | | | | grade | | | | |
| | Valid- | 3.28 | 2.22 | 0.18 | 328 | 1.30 | Serous | 12.62 | Included | Test | Benign |
| | CF40 | | | | | | cystadenoma | | | | (0.00%) |
| | Valid- | 63.88 | 1.89 | 1.64 | 6388 | 0.15 | Neuroendocrine | 2129.33 | Included | Training | Malignant |
| | CF41 | | | | | | tumor | | | | (98.25%) |
| | Valid- | 157.42 | 1.89 | 1.78 | 15742 | 0.50 | Neuroendocrine | 1574.20 | Included | Training | Malignant |
| | CF42 | | | | | | tumor | | | | (99.91%) |
| | Valid- | 13.55 | 1.9 | 0.75 | 1355 | 0.50 | Neuroendocrine | 135.50 | Included | Test | Malignant |
| | CF43 | | | | | | tumor | | | | (99.90%) |
| | Valid- | 19.22 | 1.95 | 1.17 | 1922 | 0.50 | Neuroendocrine | 192.20 | Included | Test | Malignant |
| | CF44 | | | | | | tumor | | | | (99.98%) |
| | Valid- | 724.56 | 1.93 | 1.99 | 72456 | 0.50 | Neuroendocrine | 7245.60 | Included | Test | Malignant |
| | CF45 | | | | | | tumor | | | | (71.25%) |
| | Valid- | 444.16 | 2.23 | 1.96 | 44416 | 0.50 | Solid | 4441.60 | Included | Training | Malignant |
| | CF46 | | | | | | pseudopapillary | | | | (99.88%) |
| | | | | | | | neoplasm | | | | |
| | Valid- | 40.81 | 2.31 | 1.57 | 4081 | 0.50 | Solid | 408.10 | Included | Training | Malignant |
| | CF47 | | | | | | pseudopapillary | | | | (99.92%) |
| | | | | | | | neoplasm | | | | |
| | Valid- | 73.05 | 2.36 | 1.56 | 7305 | 0.50 | Solid | 730.50 | Included | Test | Malignant |
| | CF48 | | | | | | pseudopapillary | | | | (99.82%) |
| | | | | | | | neoplasm | | | | |
| | Valid- | 8.76 | 2.76 | 0.28 | 876 | 0.50 | Solid | 87.60 | Included | Test | Malignant |
| | CF49 | | | | | | pseudopapillary | | | | (87.86%) |
| | | | | | | | neoplasm | | | | |
| | Valid- | 4.59 | 1.48 | 0.26 | 459 | 0.30 | IPMN | 76.50 | Included | Test | Malignant |
| | CF5 | | | | | | intermediate | | | | (91.78%) |
| | | | | | | | grade | | | | |
| | Valid- | 2366.69 | 6.17 | 2.12 | 236669 | 0.50 | Solid | 3666.90 | Included | Test | Malignant |
| | CF50 | | | | | | pseudopapillary | | | | (99.84%) |
| | | | | | | | neoplasm | | | | |
| | Valid- | 4.91 | 1.48 | 0.77 | 491 | 0.25 | IPMN | 98.20 | Included | Test | Malignant |
| | CF6 | | | | | | intermediate | | | | (95.46%) |
| | | | | | | | grade | | | | |
| | Valid- | 4.15 | 1.50 | 0.28 | 415 | 1.20 | IPMN | 17.29 | Included | Test | Malignant |
| | CF7 | | | | | | intermediate | | | | (100.00%) |
| | | | | | | | grade | | | | |
| | Valid- | 5.51 | 1.57 | 0.42 | 551 | 0.30 | IPMN | 91.83 | Excluded: | NA | NA |
| | CF8 | | | | | | intermediate | | median Ct >32 | | |
| | | | | | | | grade | | | | |
| | Valid- | 114.43 | 1.59 | 1.4 | 11443 | 0.50 | IPMN | 1144.30 | Included | Test | Malignant |
| | CF9 | | | | | | intermediate | | | | (80.90%) |
| | | | | | | | grade | | | | |

Laser Microdissection.

Laser microdissection of the non-invasive IPMN epithelium was performed on a PALM Micro Beam System (Carl Zeiss MicroImaging, Inc., Thornwood, N.Y.). 5-20 sections (6-10 μm thickness) were embedded onto UV-pretreated PALM® membrane slides and stained with H+E prior to laser microdissection. Where extensive papillary epithelium could be grossly identified on the slide, the neoplastic epithelium was microdissected using ultra-fine high-precision tweezers (Electron Microscopy Sciences, Hatfield, Pa.) under a stereoscopic zoom microscope SMZ1500 (Nikon, Tokyo, Japan). The number of cells harvested by microdissection from FFPE specimens ranged from 5,000-15,000 and 1,250-7,500 for the FTS1 (Disc-FT1 through FT22) and FTS2 (Valid-FT1 through FT38) specimens, respectively.

RNA Extraction from FFPE Tissue and Cyst Fluid Specimens.

Total RNA was extracted from microdissected FFPE tissues using the RecoverAll™ Total Nucleic Acid Isolation Kit for FFPE (Ambion, Austin, Tex.) according to the manufacturer's protocol. This method allows robust and reproducible recovery of RNA from FFPE tissues in sufficient quality and quantity to support miRNA expression profiling studies (Doleshal et al., 2008). To increase the final concentration for downstream applications, total RNA was eluted in 60 μl of nuclease-free (NF) water (Ambion). Total RNA was extracted from cyst fluid specimens (0.05-1.5 mL) according to Asuragen's standard operating procedures using an in-house developed mirVana™ PARIS™ Kit (Ambion)-based protocol. RNA was eluted in 100 μl of NF water. The concentration and purity were assessed with a NanoDrop 1000 spectrophotometer (NanoDrop Technologies/Thermo Scientific, Wilmington, Del.).

The average RNA recovery from cells lifted from FTS1 and FTS2 specimens was 1420 ng (range: 245-4650 ng) and 840 ng (range: 159-3552 ng), respectively.

MiRNA Expression Analyses in FFPE Tissue and Cyst Fluid Specimens.

High-throughput (HT) miRNA expression analyses were performed to identify miRNAs that distinguish between LG IPMNs and/or SCA and HG IPMNs. For FFPE Tissue Study 1 (FTS1; LG IPMN and 12 HG IPMN, see Table 1, specimens Disc-FT1-22), 750 mature miRNAs (Pool A and B) were interrogated. For CFS1 (3 LG IPMN, 5 HG IPMN and 5 SCA, see Table 2, specimens Disc-CF1-15), expression of 377 mature miRNAs (Pool A only) was examined.

10 ng of total RNA from each FFPE and cyst fluid specimen was converted into cDNA using Megaplex RT Primers (Applied Biosystems) and TaqMan miRNA RT Kits (Applied Biosystems). cDNA was pre-amplified (12 cycles) using Megaplex PreAmp Primers (Pool A and/or Pool B) prior to mixing with TaqMan Universal PCR Master Mix (Applied Biosystems) and loading onto TaqMan human miRNA fluidic cards (Applied Biosystems). The cards were run using the Applied Biosystems 7900HT real-time PCR instrument equipped with a heating block for the fluidic card (Applied Biosystems). Prior to bioinformatics analysis, raw data were processed using Relative Quantification (ΔΔCt) and the RQ Manager, with baseline set to “automatic” and Ct threshold set to 0.2.

Singleplex RT-qPCR verification was performed for the miRNA candidates. Expression levels of 26 “tissue miRNAs” and up to 37 “cyst fluid miRNAs” identified in the FTS1 and CFS1 studies, respectively, were verified in the same FFPE and cyst fluid specimen RT-qPCR was performed as follows: 10 ng total RNA was used per reverse transcription reaction (30 min, 16° C.; 30 min, 42° C.; 5 min, 85° C.; hold at 4° C.). Positive tissue QC and no-template control (NTC, nuclease-free water) samples were used to control for reagent performance and contamination. qPCR was run on the 7900HT instrument as follows: 10 min at 95° C.; 45 cycles of: 15 sec at 95° C. and 30 sec at 60° C.

Bioinformatic analyses. In brief, for the FFPE tissue study, miRNA candidates from the Megaplex/TaqMan MicroRNA Arrays were selected on the basis of strong Ct estimates (≦30), statistical tests for differential expression (TTest and Wilcox Test, FDR<0.05) and indication of a possible role in pancreatic cancer from literature and internal studies. Expression of these candidates was verified using singleplex RT-qPCR in the original samples profiled by Megaplex (n=22, FST1) and an additional set of specimens in two separate batches (n=38, FTS2), for which batch effects were ruled out prior to the analysis. miRNAs with average expression values 35 Ct across all samples were considered to be non-specifically amplified and therefore were excluded from the final data analysis (Schmittgen et al., 2008).

As a prerequisite to the screen, miRNAs were removed unless they had strong Ct estimates with both mean and median Ct values <30 based on Megaplex data. This criterium tended to remove miRNAs that had multiple non-determined calls. A priority list of candidates was ranked with a simple heuristic: the sum of the negative of the log of the FDR values from the T-Test and Wilcox-Test. In order to preclude bias that could result from the removal of miRNAs before the FDR estimates are calculated, the final selected candidates had FDR <0.05 for both tests before any filtering. Finally, additional candidates were manually selected based on publications or internal studies implicating their roles in pancreatic cancer. The short list of candidates was interrogated verified using singleplex RT-qPCR on the original samples profiled by Megaplex, plus an additional set of samples in 2 separate batches (FTS2). For evaluation of batch effects, the microdissected FFPE specimens (Table 1) were assigned to one of two groups: HG IPMNs (including specimens from Discovery FTS1 and specimens without and with cancer from the Validation FTS2) and LG IPMN (comprising specimens from Discovery FTS1 and Validation FTS2). TTest analysis showed no evidence of significant batch effect for any of the miRNAs selected for further analysis. miRNAs with the average expression value in the singleplex candidate verification (FTS1) and validation (FTS2) across all the samples >35 Ct were considered to be non-specifically amplified and therefore were excluded from the final data analysis (Akao et al., 2007).

For the cyst fluid study, bioinformatics analysis was performed on CFS1 miRNA expression data generated with the TaqMan MicroRNA Arrays platform from 4 SCA, 3 LG IPMN, and 4 HG IPMN CF specimens. miRNA candidates for further analysis (“cystic fluid miRNAs”, FIG. 1) were identified through manual selection and statistical testing for differential expression, both for individual miRNAs and DiffPair biomarkers (expression of one miRNA subtracted from that of another to generate a self-normalizing biomarker). Only the DiffPairs with FDR-adjusted p-values <0.05 and individual miRNAs with p-values <0.01 were considered for candidate verification by singleplex RT-qPCR. In addition, the miRNA candidates identified from the FTS1 and FTS2 studies were included (“verified tissue miRNAs”, FIG. 1). Differential expression analysis of 37 miRNAs yielded a shortlist of 18 miRNA candidates (Table 3, below) that comprised 27 top DiffPairs. Expression of these 18 miRNAs together with miR-21 was interrogated by singleplex RT-qPCR in an independent set of 49 CF specimens composed of 20 SCAs, 2 LG IPMNs, 11 IG IPMNs, 6 HG IPMNs, 5 NETs, and 5 SPNs (CFS2). miR-21 was included because previous experiments suggested its potential role in pancreatic carcinogenesis.

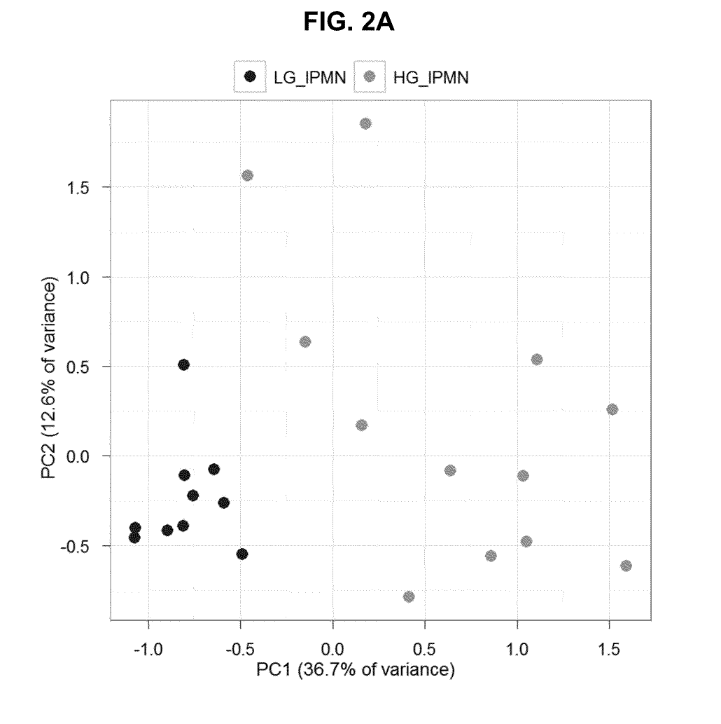

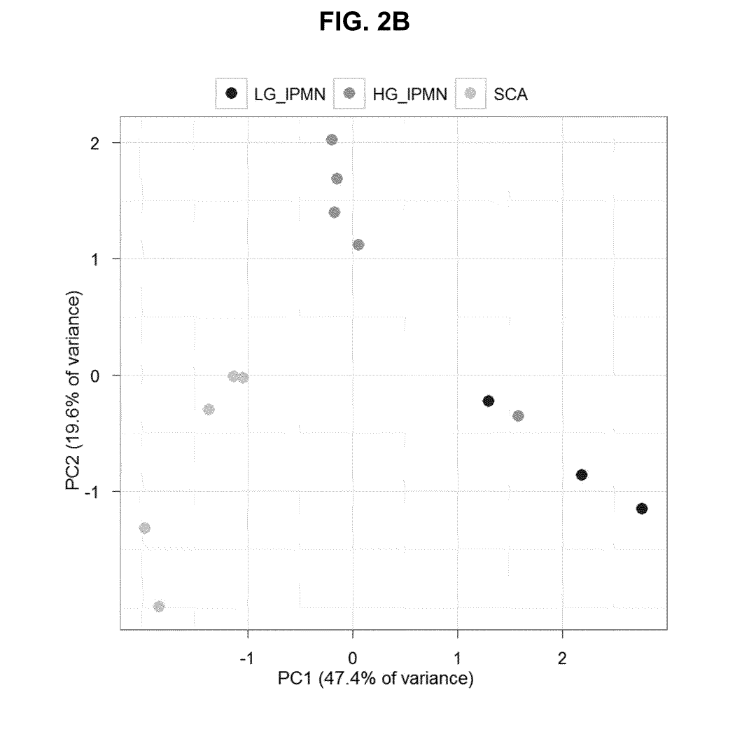

The methodology used to generate a final list of candidates was essentially a sequential set of filters. The filtering was done step-wise in order to minimize cost and time, yet keeping as broad a panel of candidates as possible. To begin, the Megaplex analysis was performed using 5 SCA, 5 LG IPMN, and 5 HG IPMN specimens. One LG IPMN specimen, Disc-CF4, was retained for the verification of candidate miRNAs in order to preserve RNA. Another, Disc-CF1, was unintentionally left out of the analysis, but when it was included towards the end of the study, it had no major impact on the shortlist of miRNA candidates generated without it. One SCA specimen (Disc-CF12, Table 2) was excluded from analysis on the basis of large number of missing Ct values (approximately 68%). One HG IPMN specimen (Disc-CF9) clustered with the LG IPMNs in an unsupervised PCA analysis (FIG. 2B). Since data analysis performed with and without Disc-CF9 showed qualitatively similar results, this specimen was removed as well. As a result, bioinformatics analysis was performed on 11 CF specimens (4 SCA, 3LG IPMN and 4 HG IPMN). Differentially expressed miRNAs from the Megaplex data were identified using TTest, while DiffPairs (expression of one miRNA subtracted from another to generate a self-normalizing biomarker) were identified with both TTest and Pearson correlation coefficients across indication categories. No Ct cutoff below the experimental Ct limit of 40 on miRNAs was incorporated into DiffPair analyses. Only those DiffPairs with FDR-adjusted p-values <0.06 were considered for subsequent analysis. Based on the Megaplex data analysis of the cyst fluid study, two candidate listings were derived. The first candidate set is derived from the top 10 DiffPairs (unadjusted p-value p<6.75×10⁻⁵) yielding 17 distinct miRNAs (Table 7, below). The second candidate set is composed of the top 10 individual miRNAs (with unadjusted p-value p<0.01) producing 5 miRNAs not present in the top 10 DiffPairs (Table 8, below). These two candidate sets produced a total of 22 miRNA candidates.

The two candidate sets based on Megaplex data analysis of cyst fluids were merged with candidates from the FFPE tissue study and other manually curated miRNAs. The merged candidate listing produced a listing of 37 miRNAs. Specifically, these 37 miRNAs were derived from the top 10 cyst fluid DiffPairs (see Table 7) combined with the top ten individual miRNAs (see Table 8), the top 13 FFPE tissue miRNAs from the 30 DiffPairs (Table 5, Table 7) and with six miRNAs (let-7b, miR-223, miR-30b, miR-328, miR-532-3p, miR-590-5p) selected manually based on their combined performance as individual candidates, in DiffPairs, and on their high expression levels. These 37 miRNAs were verified with singleplex RT-qPCR for all CFS1 samples with sufficient RNA. 11 specimens were profiled with the full 37 miRNA panel; only 13 miRNA candidates were interrogated for Disc-CF-2, and CF-4, -CF 11 and -CF14 (Table 6). Disc-CF9 and Disc-CF12 were omitted from analysis for the same reasons as described in the Megaplex analysis above.

Candidates from the CFS1 singleplex RT-qPCR data set were evaluated as DiffPairs. Expression values above 32 Ct were treated as missing in order to filter out low signal miRNAs from further consideration. Candidate DiffPairs were assessed by TTest for significant differential expression using FDR-adjusted p-value <0.05. CFS1 singleplex analysis reduced the candidate 37 miRNA set to 27 DiffPairs composed of 18 miRNAs (Table 3).

TABLE 3
MiRNA and DiffPair Candidates

| | Mean: | StDev: | Mean: | StDev: | p-value | |
|---|---|---|---|---|---|---|
| Biomarker | LG/SCA | LG/SCA | HG | Hg | (T-Test) | FDR p-value |
| Diff(miR-106b, | −3.87 | 0.15 | −6.66 | 0.2 | 1.10E−06 | 0.00058 |
| miR-642) | | | | | | |
| Diff(miR-24, | −2.81 | 0.95 | −5.55 | 0.5 | 6.96E−05 | 0.01837 |
| miR-331) | | | | | | |
| Diff(let-7b, | −8.98 | 0.57 | −5.61 | 0.66 | 2.64E−04 | 0.03702 |
| miR-18a) | | | | | | |
| Diff(miR-15b, | −0.91 | 1.32 | −5.15 | 0.87 | 2.80E−04 | 0.03702 |
| miR-99b) | | | | | | |
| Diff(miR-92a, | −2.02 | 0.42 | −0.69 | 0.03 | 5.76E−04 | 0.03711 |
| miR-93) | | | | | | |
| Diff(miR-142- | 0.96 | 1.52 | −5.85 | 1.42 | 6.16E−04 | 0.03711 |
| 3p, miR-99b) | | | | | | |
| Diff(let-7b, | −3.92 | 0.62 | −1.24 | 0.67 | 6.31E−04 | 0.03711 |
| miR-93) | | | | | | |
| Diff(let-7b, | −5.52 | 0.75 | −2.78 | 0.71 | 6.84E−04 | 0.03711 |
| miR-15b) | | | | | | |
| Diff(miR-106b, | −0.65 | 0.9 | −3.85 | 0.84 | 6.85E−04 | 0.03711 |
| miR-331) | | | | | | |
| Diff(let-7b, | −5.77 | 0.89 | −3.07 | 0.53 | 9.59E−04 | 0.03711 |
| miR-106b) | | | | | | |
| Diff(miR-142- | 0.54 | 1.9 | −3.92 | 1.17 | 1.00E−03 | 0.03711 |
| 3p, miR-342- | | | | | | |
| 3p) | | | | | | |
| Diff(miR-106b, | −0.79 | 0.4 | −4.71 | 0.86 | 1.06E−03 | 0.03711 |
| miR-30a-3p) | | | | | | |
| Diff(miR-15b, | 1.25 | 1.01 | −1.2 | 0.35 | 1.10E−03 | 0.03711 |
| miR-34a) | | | | | | |
| Diff(miR-34a, | 0.75 | 1.18 | 2.74 | 0.48 | 1.23E−03 | 0.03711 |
| miR-93) | | | | | | |
| Diff(miR-15b, | −0.74 | 1.17 | −4.13 | 0.75 | 1.28E−03 | 0.03711 |
| miR-331) | | | | | | |
| Diff(miR-15b, | −0.75 | 0.66 | −4.99 | 1.09 | 1.31E−03 | 0.03711 |
| miR-30a-3p) | | | | | | |
| Diff(miR-142- | 0.83 | 1.54 | −5.68 | 1.7 | 1.32E−03 | 0.03711 |
| 3p, miR-30a- | | | | | | |
| 3p) | | | | | | |
| Diff(miR-142- | 0.18 | 1.79 | −4.83 | 1.48 | 1.32E−03 | 0.03711 |
| 3p, miR-331) | | | | | | |
| Diff(miR-15b, | −1.45 | 0.87 | −3.94 | 0.54 | 1.34E−03 | 0.03711 |
| miR-532-3p) | | | | | | |
| Diff(miR-99b, | 2.51 | 1.25 | 6.69 | 1.26 | 1.60E−03 | 0.04129 |
| miR-93) | | | | | | |
| Diff(miR-142- | −0.33 | 1.46 | −6.42 | 1.68 | 1.66E−03 | 0.04129 |
| 3p, miR-328) | | | | | | |
| Diff(miR-24, | −2.84 | 1.63 | −6.57 | 0.45 | 1.72E−03 | 0.04129 |
| miR-99b) | | | | | | |
| Diff(let-7c, | −3.08 | 0.67 | −0.58 | 0.33 | 1.92E−03 | 0.04303 |
| miR-18a) | | | | | | |
| Diff(miR-18a, | 1.4 | 0.45 | −1.12 | 0.73 | 2.02E−03 | 0.04303 |
| miR-532-3p) | | | | | | |
| Diff(miR-223, | −0.65 | 2.43 | −9.25 | 2.69 | 2.04E−03 | 0.04303 |
| miR-99b) | | | | | | |
| Diff(let-7c, | 1.73 | 0.55 | 3.79 | 0.63 | 2.19E−03 | 0.04373 |
| miR-93) | | | | | | |
| Diff(miR-642, | 5.35 | 0.9 | 8.49 | 0.83 | 2.24E−03 | 0.04373 |
| miR-93) | | | | | | |

Expression of these 18 miRNAs together with miR-21 was further evaluated by singleplex RT-qPCR in an independent set of 50 CF specimens (CFS2). One IG IPMN specimen (Valid-CF8) was excluded due to high Ct values (median Ct >32), leaving 49 samples for further analysis. These samples, along with the CFS1 specimens, were used to develop and assess a logistic regression model for sample classification as described below.

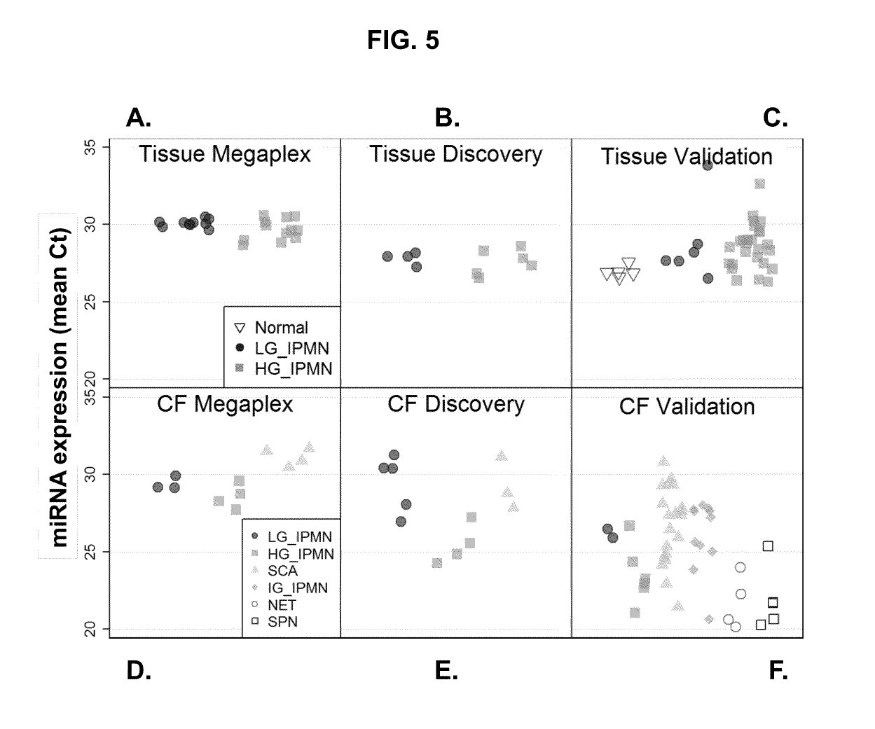

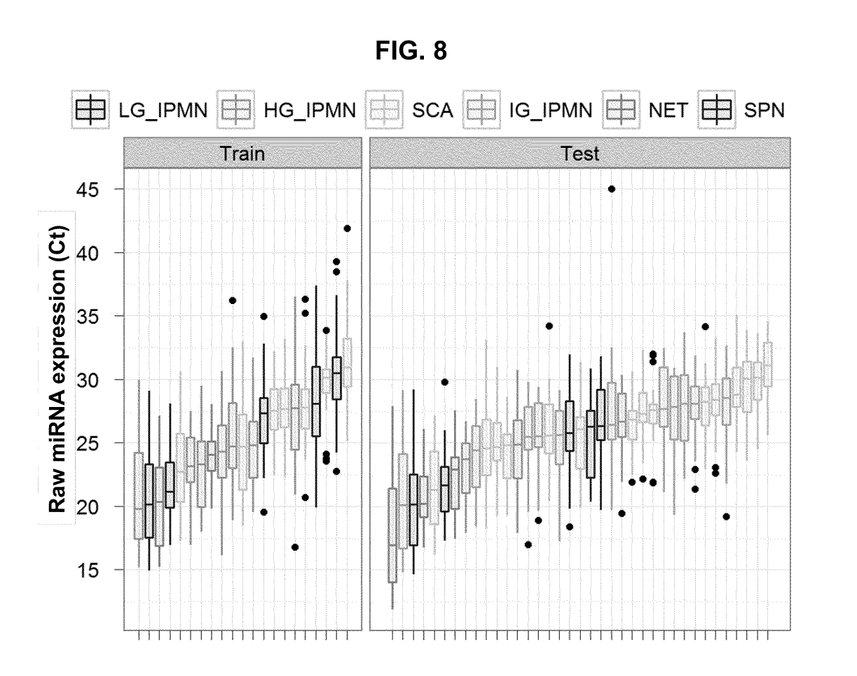

Of note, correlation between mean Ct from an array and diagnosis was observed for cyst fluid samples, as shown by ANOVA yielding R2 values of 0.81 for CF Discovery Megaplex (CFS1), 0.63 for CF Discovery singleplex (CFS1), and 0.49 for CF Validation (CFS2) (all significant at p<0.05 level) (FIG. 5, FIG. 8).

Logistic Regression Model to Guide Resection.

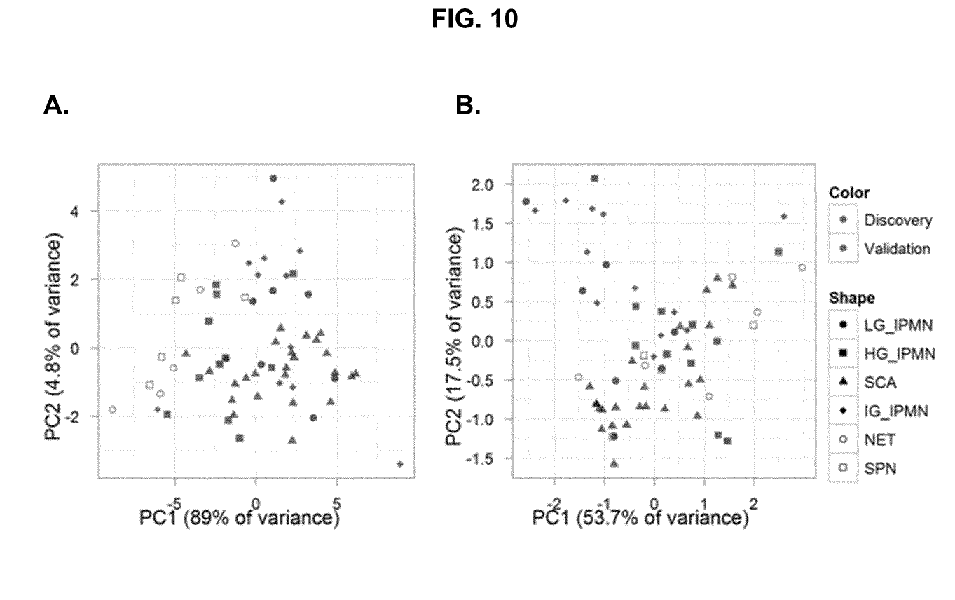

The singleplex RT-qPCR expression data for the 18 cyst fluid miRNAs (Table 3) and miR-21 generated from 9 CFS1 and 49 CFS2 specimens were merged together. No apparent batch effects were observed between the two specimen sets (FIG. 10). The CFS1 and CFS2 specimens were merged together and then split into a training and test set as detailed in Table 2. It is important to note that no samples used in the candidate generation set (CFS1) are included in the test set for establishing model performance. The 4 specimens with incomplete RT-qPCR data for 13 miRNAs (Disc-CF2, -CF4, -CF11 and -CF14) were not used in training or testing of the logistic regression model. Disc-CF9 and Disc-CF12 were also not used for the same reasons as described above. The separation of samples into training and test sets was based on order of isolation.

SCAs were grouped with LG IPMNs, because both do not need to be resected unless symptomatic (this group is referred to as “benign,” which means low risk lesion), and HG IPMNs were grouped with SPNs and NETs, due to their high potential for malignancy and common treatment by surgery (this group is referred to as “malignant,” which refers to a high risk lesion). The 20 DiffPairs found to be most differentially expressed between benign and malignant specimens in the merged CSF1/CSF2 data set were used as predictors for an L1-penalized logistic model for distinguishing between benign and malignant.

The first round of feature selection was conducted through pairwise comparison of DiffPair ΔCt values in benign (LG IPMN/SCA) as compared to malignant (HG IPMN/NET/SPN) samples on the 21 training setspecimens (Michael et al., 2003). Despite the lack of apparent batch effects, a two-way ANOVA model (without interactions) was fit to the data for each gene, with discovery/validation batch as one factor and resection status as the other. P-values were obtained for malignant-versus-benign ANOVA contrasts testing null hypotheses of no difference between benign and malignant groups (see Table 4). The 20 DiffPairs found to be most differentially expressed in this analysis were used as predictors for a logistic model for distinguishing malignancy status. The logistic model was fit to the 20 selected DiffPairs using L1-penalized regression, with the penalty parameter manually optimized (final value λ1=0.1 using variance standardization for predictors) through leave-one-out cross-validation using the cvl and penalized functions in the R penalized package (Lanza et al., 2007). Manual optimization of cross-validated log-likelihood (CVL) was used because of numerical difficulties encountered with the automatic optL1 function for penalty parameter estimation: CVL was essentially constant near maximum value for λ1 between 0.05 and 0.15. The parameters of the resulting model are shown in Table 4. Only 7 of the 20 predictor DiffPairs received non-zero weights due to the L1-penalty applied during the fitting process. The weights of the DiffPairs in the regression model are not surprisingly correlated with the ANOVA contrast p-values. In particular, the DiffPair with the lowest ANOVA contrast p-value, Diff(miR-24, miR-30a-3p) also received the largest weight in the regression model, with 5 of the 7 non-zero regression weighted DiffPairs appearing in the top 10 DiffPairs by ANOVA contrast p-value.

TABLE 4
Weight Parameters for Logistic Model

| | | | | Absolute |
|---|---|---|---|---|
| | | | | Value |
| | p-value | FDR p- | Regression | Regression |
| Biomarker | (ANOVA) | value | Coefficient | Coefficient |
| Diff(hsa-miR-24, | 6.78E−06 | 0.0010 | 1.69 | 1.69 |
| hsa-miR-30a-3p) | | | | |
| Diff(hsa-miR-18a, | 3.92E−04 | 0.0120 | 1.47 | 1.47 |
| hsa-miR-92a) | | | | |
| Diff(hsa-miR-24, | 4.12E−03 | 0.0351 | 0.74 | 0.74 |
| hsa-miR-342-3p) | | | | |
| Diff(hsa-miR-24, | 1.60E−04 | 0.0061 | 0.69 | 0.69 |
| hsa-miR-99b) | | | | |
| Diff(hsa-miR-106b, | 1.26E−03 | 0.0241 | 0.42 | 0.42 |
| hsa-miR-92a) | | | | |
| Diff(hsa-miR-142-3p, | 3.03E−03 | 0.0289 | 0.18 | 0.18 |
| hsa-miR-92a) | | | | |
| Diff(hsa-miR-30a-3p, | 1.19E−03 | 0.0241 | −0.16 | 0.16 |
| hsa-miR-532-3p) | | | | |
| Diff(hsa-miR-92a, | 1.09E−04 | 0.0061 | 0.00 | 0 |
| mmu-miR-93) | | | | |
| Diff(hsa-miR-24, | 1.42E−04 | 0.0061 | 0.00 | 0 |
| hsa-miR-328) | | | | |
| Diff(hsa-miR-24, | 1.01E−03 | 0.0241 | 0.00 | 0 |
| hsa-miR-331) | | | | |
| Diff(hsa-miR-106b, | 1.59E−03 | 0.0265 | 0.00 | 0 |
| hsa-miR-30a-3p) | | | | |
| Diff(hsa-miR-30a-3p, | 1.73E−03 | 0.0265 | 0.00 | 0 |
| hsa-miR-34a) | | | | |
| Diff(hsa-let-7b, | 2.07E−03 | 0.0265 | 0.00 | 0 |
| hsa-miR-106b) | | | | |
| Diff(hsa-miR-142-3p, | 2.08E−03 | 0.0265 | 0.00 | 0 |
| hsa-miR-30a-3p) | | | | |
| Diff(hsa-let-7b, | 2.47E−03 | 0.0289 | 0.00 | 0 |
| mmu-miR-93) | | | | |
| Diff(hsa-miR-18a, | 3.00E−03 | 0.0289 | 0.00 | 0 |
| hsa-miR-30a-3p) | | | | |
| Diff(hsa-let-7b, | 3.16E−03 | 0.0289 | 0.00 | 0 |
| hsa-miR-18a) | | | | |
| Diff(hsa-miR-30a-3p, | 3.21E−03 | 0.0289 | 0.00 | 0 |
| mmu-miR-93) | | | | |
| Diff(hsa-miR-142-3p, | 4.77E−03 | 0.0384 | 0.00 | 0 |
| hsa-miR-328) | | | | |
| Diff(hsa-miR-15b, | 5.94E−03 | 0.0454 | 0.00 | 0 |
| hsa-miR-92a) | | | | |

### Results

Microdissected FFPE Specimens.

Initial expression profiling of 750 mature miRNAs was performed in microdissected cell populations from 10 LG IPMNs and 12 HG IPMNs with and without associated invasive carcinoma (FTS1, Table 1). Use of multiplex RT and cDNA pre-amplification allowed significant reduction of the RNA input relative to singleplex RT-qPCR. Data from Asuragen (unpublished) and other research groups show that pre-amplification of miRNA-containing cDNA improves sensitivity of miRNA detection, while maintaining the relative expression levels (Mestdagh et al., 2008; Chen et al., 2009). Clear separation between experimental groups was observed (FIG. 2A). The bioinformatics analysis produced 26 miRNA candidates including: miR-100, miR-106b, miR-125b, miR-139-5p, miR-145, miR-150, miR-151-3p, miR-17, miR-196a, miR-200a, miR-200b, miR-20b, miR-210, miR-214, miR-217, miR-26a, miR-28-5p, miR-30a-3p, miR-30e-3p, miR-342-3p, miR-34a, miR-375, miR-660, miR-93, miR-99a and let-7c.

Next, the 26 miRNA candidates identified via Megaplex expression analysis were verified using singleplex RT-qPCR. Due to insufficient RNA yield, only 10 out of 12 specimens were evaluated, including 4 LG IPMNs (Disc-FT3, 4, 5 and 9) and 6 HG IPMNs (Disc-FT12, 13, 17, 21, 22, 23). Additionally, expression of these miRNAs was validated in an independent set of 28 FFPE specimens composed of 5 normal (Valid-FT34-38), 3 LG IPMN (Valid-FT15, 24, 27), 9 HG IPMN (Valid-FT1, 4, 11, 13, 14, 17, 18, 25 and 28) and 11 HG IPMN with associated invasive carcinoma (Valid-FT3, 7 to 10, 12, 16, 26, 29, 31, and 32). In the remaining 10 samples, mean Ct estimates exceeded 30 or limited RNA was available for interrogation of only the following 13 miRNAs: miR-196a, miR-217, miR-210, miR-375, miR-30a-3p, miR-106b, miR-17, miR-150, miR-26a, miR-99a, miR-125b, miR-20b and miR-93. The first 4 miRNAs were included in this test set due to their association with pancreatic cancer. The remaining 9 miRNAs were the top miRNA candidates selected from the set of 26 “tissue miRNAs”. Three specimens (Valid-FT2, -FT5 and -FT6) were excluded from further analysis based on a high percentage of missing values or outlying mean Ct within the diagnostic group.

For final selection and ranking of miRNA candidates that distinguish between LG IPMNs/SCAs and HG IPMNs (with and without invasive carcinoma), all singleplex RT-qPCR data generated from the FTS1 and FTS2 studies (51 specimens) were combined. Evaluation of batch effects rendered no trend associated with batching. Differential expression analysis between LG IPMN/SCA and HG IPMN specimens was conducted. Thirty significant DiffPairs composed of 13 miRNAs were identified for further investigation in the cyst fluid study (Table 5 and Table 6). These candidates represent miRNAs that could be excreted into cyst fluid in sloughed off neoplastic epithelium cells. Table 5 provides 30 DiffPairs comprised of 13 miRNAs (“verified tissue miRNAs”) identified from FTS1 and FTS2 specimens for further investigation in cyst fluid specimens. Table 6 provides the 37 miRNAcyst fluid candidates from the CFS1 data for further singleplex RT-qPCR verification.

TABLE 5
DiffPairs Identified for Further Investigation (in CFS)

| | | | | | | | FDR p- |
|---|---|---|---|---|---|---|---|
| | | | | | | | value |
| | Mean: | StDev: | Mean: | StDev: | p-value | FDR p-value | (Rank |
| Biomarker | LG | LG | HG | HG | (T-Test) | (T-Test) | Product) |
| Diff(miR- | 2.27 | 0.7 | −0.8 | 2.01 | 0.00042 | 0.035 | 0.0032 |
| 106b, miR- | | | | | | | |
| 99a) | | | | | | | |
| Diff(miR-210, | 1.75 | 1.02 | −1.25 | 2 | 0.00062 | 0.035 | 0.0000 |
| miR-99a) | | | | | | | |
| Diff(miR-93, | −0.11 | 0.53 | −3.11 | 2.07 | 0.00068 | 0.035 | 0.0020 |
| miR-99a) | | | | | | | |
| Diff(miR-17, | 0.95 | 0.65 | −2.04 | 2.13 | 0.00099 | 0.035 | 0.0034 |
| miR-99a) | | | | | | | |
| Diff(miR-20b, | 7.19 | 0.54 | 3.78 | 2.53 | 0.00141 | 0.035 | 0.0000 |
| miR-99a) | | | | | | | |
| Diff(miR-100, | −2.46 | 0.67 | 0.41 | 2.13 | 0.00150 | 0.035 | 0.0163 |
| miR-106b) | | | | | | | |
| Diff(miR- | 2.79 | 0.69 | 0.03 | 2.1 | 0.00190 | 0.035 | 0.0058 |
| 106b, miR- | | | | | | | |
| 150) | | | | | | | |
| Diff(miR-100, | −1.94 | 1.07 | 0.86 | 2.12 | 0.00217 | 0.035 | 0.0072 |
| miR-210) | | | | | | | |
| Diff(miR-100, | −0.08 | 0.61 | 2.72 | 2.18 | 0.00226 | 0.035 | 0.0137 |
| miR-93) | | | | | | | |
| Diff(miR-150, | −1.47 | 0.67 | 1.21 | 2.13 | 0.00273 | 0.035 | 0.0202 |
| miR-17) | | | | | | | |
| Diff(miR-150, | −0.41 | 0.56 | 2.29 | 2.15 | 0.00275 | 0.035 | 0.0169 |
| miR-93) | | | | | | | |
| Diff(let-7c, | −1.26 | 0.72 | 1.31 | 2.04 | 0.00283 | 0.035 | 0.0402 |
| miR-106b) | | | | | | | |
| Diff(miR-20b, | 5.17 | 0.33 | 2.6 | 2.09 | 0.00304 | 0.035 | 0.0395 |
| miR-342-3p) | | | | | | | |
| Diff(miR- | 4.72 | 0.77 | 1.92 | 2.25 | 0.00309 | 0.035 | 0.0033 |
| 106b, miR- | | | | | | | |
| 125b) | | | | | | | |
| Diff(miR-100, | −1.14 | 0.69 | 1.64 | 2.25 | 0.00313 | 0.035 | 0.0168 |
| miR-17) | | | | | | | |
| Diff(miR-150, | −2.27 | 0.96 | 0.43 | 2.17 | 0.00339 | 0.035 | 0.0177 |
| miR-210) | | | | | | | |
| Diff(miR-100, | −7.37 | 0.66 | −4.18 | 2.62 | 0.00344 | 0.035 | 0.0000 |
| miR-20b) | | | | | | | |
| Diff(miR-660, | 3.03 | 0.84 | 0.28 | 2.25 | 0.00376 | 0.035 | 0.0027 |
| miR-99a) | | | | | | | |
| Diff(let-7c, | 1.12 | 0.73 | 3.62 | 2.09 | 0.00429 | 0.036 | 0.0276 |
| miR-93) | | | | | | | |
| Diff(miR- | −4.2 | 1.1 | −1.47 | 2.26 | 0.00441 | 0.036 | 0.0087 |
| 125b, miR- | | | | | | | |
| 210) | | | | | | | |
| Diff(miR-210, | −0.26 | 0.8 | −2.44 | 1.81 | 0.00447 | 0.036 | 0.0497 |
| miR-342-3p) | | | | | | | |
| Diff(let-7c, | −0.75 | 1.09 | 1.76 | 2.1 | 0.00496 | 0.036 | 0.0144 |
| miR-210) | | | | | | | |
| Diff(miR- | −2.34 | 0.77 | 0.39 | 2.33 | 0.00499 | 0.036 | 0.0175 |
| 125b, miR- | | | | | | | |
| 93) | | | | | | | |
| Diff(miR- | −3.4 | 0.72 | −0.68 | 2.39 | 0.00609 | 0.041 | 0.0172 |
| 125b, miR- | | | | | | | |
| 17) | | | | | | | |
| Diff(miR- | −9.64 | 0.78 | −6.5 | 2.76 | 0.00616 | 0.041 | 0.0000 |
| 125b, miR- | | | | | | | |
| 20b) | | | | | | | |
| Diff(let-7c, | 0.05 | 0.76 | 2.54 | 2.2 | 0.00657 | 0.042 | 0.0440 |
| miR-17) | | | | | | | |
| Diff(let-7c, | −6.18 | 0.83 | −3.28 | 2.58 | 0.00675 | 0.042 | 0.0080 |
| miR-20b) | | | | | | | |
| Diff(miR- | −0.68 | 1.25 | −3.15 | 2.26 | 0.00822 | 0.045 | 0.0255 |
| 106b, miR- | | | | | | | |
| 30a-3p) | | | | | | | |
| Diff(miR-20b, | 4.24 | 0.71 | 1.44 | 2.61 | 0.00907 | 0.044 | 0.0035 |
| miR-30a-3p) | | | | | | | |
| Diff(miR-100, | −3.21 | 0.89 | −0.68 | 2.37 | 0.00976 | 0.045 | 0.0131 |
| miR-660) | | | | | | | |

TABLE 6
Prioritization of Identified DiffPairs (for CFS)

| | | CF | CF | Manual | High |
|---|---|---|---|---|---|
| miRNA | FFPE | DiffPair | Individual | Selection | Priority |
| let-7b | | | | X | |
| let-7c | X | X | | | X |
| miR-100 | X | | | | |
| miR-106b | X | X | | | X |
| miR-125b | X | | | | |
| miR-142-3p | | X | X | | X |
| miR-150 | X | | | | |
| miR-15b | | | X | | |
| miR-17 | X | | | | |
| miR-185 | | X | | | X |
| miR-18a | | | X | | |
| miR-195 | | X | | | X |
| miR-197 | | | X | | |
| miR-199a-3p | | X | | | |
| miR-20b | X | | | | |
| miR-210 | X | | | | |
| miR-223 | | | | X | |
| miR-24 | | X | | | X |
| miR-28 | | X (28-5p) | | | X (28-5p) |
| miR-301 | | X (301a) | | | |
| miR-30a-3p | X | | | | |
| miR-30b | | | | X | |
| miR-328 | | | | X | |
| miR-331 | | X (331-3p) | X (331-3p) | | X (331-3p) |
| miR-342-3p | X | X | | | X |
| miR-34a | | | X | | X |
| miR-455 | | | X (455-5p) | | |
| miR-489 | | X | | | |
| miR-532-3p | | | | X | |
| miR-590-5p | | | | X | |
| miR-597 | | X | X | | X |
| miR-642 | | X | X | | |
| miR-660 | X | | | | |
| miR-92a | | X | | | |
| miR-93 | X | X | | | X |
| miR-99a | X | | | | X |
| miR-99b | | X | X | | |

Cyst Fluid Specimens.

To improve recovery of small RNAs from cyst fluid specimens, a mirVana™ PARIS™ Kit (Ambion)-based method was developed and optimized. Low RNA recovery was observed from SCAs, LG and IG IPMNs with an average of 96 ng (range: 8.7-474 ng), 284 ng (range: 13.7-1320 ng) and 312 ng CF (range: 11.6-1456 ng) per 50 μl CF, respectively. All remaining diagnostic groups (HG IPMNs −/+carcinoma, NETs, SPNs) yielded RNA in excess of 1,400 ng/50 μl CF (range: 52.3-23,666 ng). The yield did not correlate with the presence of blood (as determined by the color of the specimen), but it did correlate with the presence of macroscopic tissue debris. Cyst fluid samples that were “cloudy” in appearance yielded far more RNA than those that appeared “watery” (as seen for SCAs). Agilent Bioanalyzer analysis of cyst fluid RNA showed no distinct 18S and 28S peaks and an average RNA fragment size <100 nt, likely due to the storage as frozen without addition of any preservation medium to attenuate the activity of RNases. Based on the demonstrated stability of miRNAs in “compromised” biospecimens (Habbe et al., 2009; Doleshal et al., 2008; Szafranska et al., 2008), their recovery from CF was not affected as determined via RT-qPCR analysis of selected miRNAs known for their abundant expression in human biofluids.

High throughput expression profiling of 377 human miRNAs (Megaplex Pool A) was carried out using CF specimens collected from patients with histologically confirmed LG IPMNs (n=3), HG IPMNs (+/−invasive carcinoma; n=4) and SCAs (n=4) (CST1; Table 2). SCAs were included because they represent cystic lesions that are entirely benign, but are sometimes misdiagnosed as IPMNs (and vice versa). Clear separation between experimental groups was observed (FIG. 2B). Similar to FFPE specimens, cyst fluid miRNA candidates were evaluated as DiffPairs. The specimens were grouped as HG IPMNs and compared to LG IPMNs combined with SCAs to identify differentially expressed candidates. Due to very few DiffPairs with p-values <0.05 (n=5, Table 7), 5 additional DiffPairs with FDR p-values ranging from 0.05-0.06 (inclusive) were selected on the basis of unadjusted p-value ranking Table 7 provides the top DiffPairs identified to T-Test p-value from the CFS1 megaplex data set, and Table 8 provides the top 10 miRNAs identified by T-Test p-value from the CFS1 Megaplex data set.

TABLE 7
Statistical Analysis of DiffPairs from the CFS1 Megaplex Data Set

| | Mean: | StDev: | Mean: | StDev: | p-value | FDR p- |
|---|---|---|---|---|---|---|
| Biomarker | LG/SCA | LG/SCA | HG | HG | (T-Test) | value |
| Diff(miR-199a-3p, | 1.21 | 1.62 | −5.75 | 0.79 | 5.50E−06 | 0.0475 |
| miR-489) | | | | | | |
| Diff(miR-185, miR- | 1.07 | 0.97 | −3.84 | 0.76 | 1.69E−05 | 0.0475 |
| 597) | | | | | | |
| Diff(miR-24, miR- | −2.58 | 0.65 | −5.19 | 0.41 | 2.08E−05 | 0.0475 |
| 331-3p) | | | | | | |
| Diff(miR-99b, miR- | −3.31 | 0.93 | 1.07 | 0.71 | 2.27E−05 | 0.0475 |
| 301a) | | | | | | |
| Diff(miR-195, miR- | −2.06 | 1.39 | −7.02 | 0.53 | 2.33E−05 | 0.0475 |
| 597) | | | | | | |
| Diff(let-7c, miR-195) | 1.49 | 0.69 | 4.90 | 0.57 | 3.42E−05 | 0.0506 |
| Diff(miR-28-5p, miR- | 2.92 | 0.23 | 0.75 | 0.29 | 3.47E−05 | 0.0506 |
| 331-3p) | | | | | | |
| Diff(miR-142-3p, | 4.24 | 2.09 | −2.81 | 1.21 | 5.83E−05 | 0.0597 |
| miR-342-3p) | | | | | | |
| Diff(miR-106b, miR- | −3.16 | 0.86 | −5.90 | 0.28 | 6.13E−05 | 0.0597 |
| 642) | | | | | | |
| Diff(miR-92a, miR- | −2.64 | 0.52 | −0.98 | 0.27 | 6.75E−05 | 0.0597 |
| 93) | | | | | | |
| Diff(miR-142-3p, | −1.39 | 1.63 | −8.80 | 1.38 | 6.78E−05 | 0.0597 |
| miR-202) | | | | | | |
| Diff(miR-145, miR- | −2.47 | 2.10 | −8.87 | 0.88 | 7.38E−05 | 0.0597 |
| 489) | | | | | | |
| Diff(miR-142-3p, | −5.04 | 2.20 | −12.73 | 1.46 | 8.42E−05 | 0.0597 |
| miR-518d-3p) | | | | | | |
| Diff(miR-28-5p, miR- | 6.56 | 0.36 | 4.75 | 0.34 | 8.92E−05 | 0.0597 |
| 30c) | | | | | | |
| Diff(miR-29b, miR- | 4.35 | 1.04 | 1.26 | 0.44 | 9.12E−05 | 0.0597 |
| 331-3p) | | | | | | |
| Diff(miR-19a, miR- | 4.21 | 0.68 | 2.17 | 0.33 | 9.45E−05 | 0.0597 |
| 92a) | | | | | | |
| Diff(miR-135a, miR- | −0.50 | 0.84 | 1.96 | 0.42 | 1.16E−04 | 0.0597 |
| 590-5p) | | | | | | |
| Diff(miR-454, miR- | 1.76 | 1.26 | −2.48 | 0.85 | 1.17E−04 | 0.0597 |
| 642) | | | | | | |
| Diff(miR-28-3p, miR- | 2.99 | 0.47 | 4.34 | 0.18 | 1.20E−04 | 0.0597 |
| 191) | | | | | | |
| Diff(miR-142-3p, | −4.39 | 1.01 | −9.64 | 1.02 | 1.31E−04 | 0.0597 |
| miR-548d-5p) | | | | | | |
| Diff(miR-331-3p, | −5.21 | 1.05 | −2.31 | 0.47 | 1.58E−04 | 0.0597 |
| miR-744) | | | | | | |
| Diff(miR-21, miR- | −8.29 | 0.77 | −6.11 | 0.40 | 1.59E−04 | 0.0597 |
| 101) | | | | | | |
| Diff(miR-150, miR- | −3.87 | 1.23 | −7.23 | 0.54 | 1.66E−04 | 0.0597 |
| 597) | | | | | | |
| Diff(miR-15b, let-7b) | 5.09 | 1.34 | 1.19 | 0.76 | 1.68E−04 | 0.0597 |
| Diff(miR-195, miR- | 1.27 | 1.42 | −2.64 | 0.68 | 1.76E−04 | 0.0597 |
| 335) | | | | | | |
| Diff(miR-28-3p, miR- | −4.01 | 1.06 | 0.19 | 0.90 | 1.80E−04 | 0.0597 |
| 454) | | | | | | |
| Diff(miR-101, miR- | 2.65 | 1.01 | −0.07 | 0.42 | 1.81E−04 | 0.0597 |
| 335) | | | | | | |
| Diff(miR-16, miR- | −2.72 | 1.41 | −6.48 | 0.52 | 1.89E−04 | 0.0597 |
| 335) | | | | | | |
| Diff(miR-125b, miR- | −5.44 | 2.12 | 0.24 | 0.81 | 1.91E−04 | 0.0597 |
| 199a-3p) | | | | | | |
| Diff(let-7g, miR-455- | −2.36 | 0.55 | −4.15 | 0.39 | 2.00E−04 | 0.0597 |
| 5p) | | | | | | |
| Diff(miR-19a, miR- | 1.36 | 1.07 | −1.79 | 0.11 | 2.16E−04 | 0.0597 |
| 335) | | | | | | |
| Diff(miR-142-3p, | 3.67 | 1.76 | −3.56 | 1.59 | 2.21E−04 | 0.0597 |
| miR-328) | | | | | | |
| Diff(miR-99b, miR- | −1.82 | 2.54 | 6.52 | 1.85 | 2.21E−04 | 0.0597 |
| 142-3p) | | | | | | |
| Diff(miR-331-3p, | −6.24 | 1.43 | −2.49 | 0.53 | 2.23E−04 | 0.0597 |
| miR-494) | | | | | | |
| Diff(miR-21, miR- | −8.99 | 1.06 | −5.99 | 0.61 | 2.27E−04 | 0.0597 |
| 340) | | | | | | |
| Diff(miR-15b, miR- | 2.74 | 1.62 | −1.97 | 1.00 | 2.30E−04 | 0.0597 |
| 331-3p) | | | | | | |
| Diff(miR-28-3p, miR- | −1.39 | 1.04 | 1.44 | 0.56 | 2.42E−04 | 0.0597 |
| 140-5p) | | | | | | |
| Diff(miR-16, miR- | −6.04 | 1.49 | −10.85 | 1.08 | 2.44E−04 | 0.0597 |
| 597) | | | | | | |
| Diff(miR-93, miR- | −0.21 | 1.06 | −2.98 | 0.30 | 2.46E−04 | 0.0597 |
| 335) | | | | | | |
| Diff(miR-34a, miR- | −4.61 | 1.23 | −1.36 | 0.32 | 2.52E−04 | 0.0597 |
| 590-5p) | | | | | | |
| Diff(miR-15b, miR- | 1.48 | 1.43 | −2.53 | 0.84 | 2.53E−04 | 0.0597 |
| 532-3p) | | | | | | |
| Diff(miR-30c, miR- | −8.79 | 1.34 | −4.23 | 1.04 | 2.54E−04 | 0.0597 |
| 454) | | | | | | |
| Diff(miR-21, miR- | −10.21 | 1.32 | −6.67 | 0.70 | 2.55E−04 | 0.0597 |
| 652) | | | | | | |
| Diff(miR-15b, miR- | 0.06 | 1.76 | −4.45 | 0.63 | 2.58E−04 | 0.0597 |
| 455-5p) | | | | | | |

TABLE 8
Top MiRNAs from the CFS1 Megaplex Data Set

| | Mean: | | | | | |
|---|---|---|---|---|---|---|
| | LG/ | StDev: | Mean: | StDev: | p-value | FDR |
| miRNA | SCA | LG/SCA | HG | HG | (T-Test) | p-value |
| miR-99b | 0.29 | 0.98 | 3.05 | 0.92 | 0.00260 | 0.109 |
| miR-597 | 1.19 | 1.51 | 4.14 | 0.96 | 0.00349 | 0.109 |
| miR-642 | 0.81 | 0.90 | 2.46 | 0.54 | 0.00433 | 0.109 |
| miR-455-5p | 0.22 | 1.02 | 2.24 | 0.74 | 0.00514 | 0.109 |
| miR-331-3p | −2.46 | 0.90 | −0.24 | 0.85 | 0.00517 | 0.109 |
| miR-142-3p | 2.12 | 3.01 | −3.47 | 2.04 | 0.00576 | 0.109 |
| miR-34a | −2.72 | 0.55 | −1.57 | 0.44 | 0.00593 | 0.109 |
| miR-18a | 4.51 | 2.77 | 0.18 | 1.17 | 0.00602 | 0.109 |
| miR-15b | 0.28 | 1.71 | −2.22 | 0.22 | 0.00815 | 0.120 |
| miR-197 | −0.42 | 1.27 | 2.19 | 1.09 | 0.00830 | 0.120 |

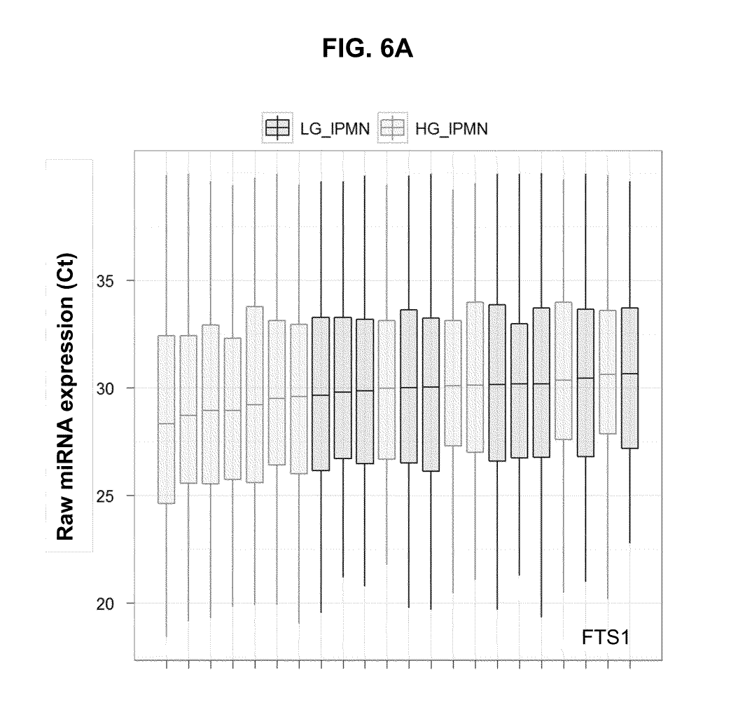

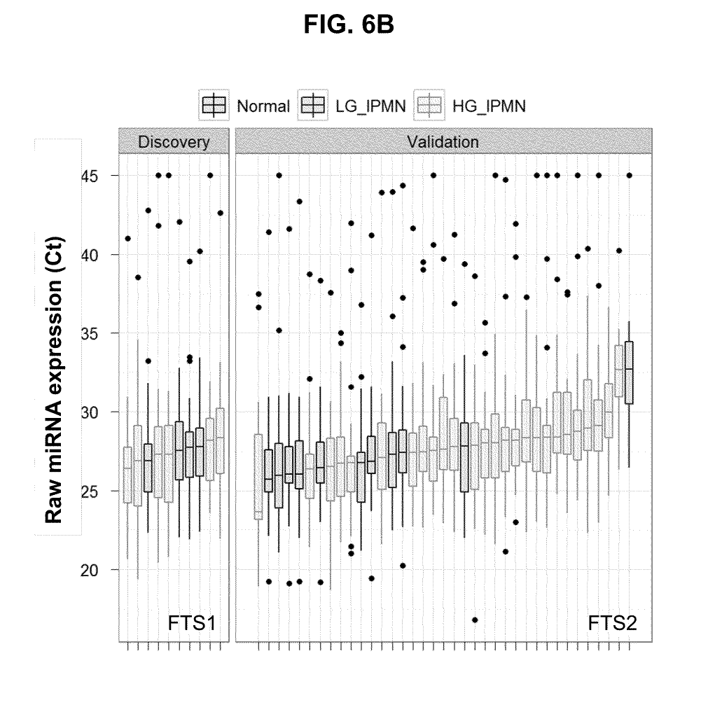

For candidate verification using singleplex RT-qPCR, 37 top differentially expressed miRNAs were chosen from three sources: FTS, CFS and expert manual selection. Four miRNAs (let-7c, miR-106b, -342-3p and -93) were significantly differentially expressed in both the FTS and the CFS candidate biomarker studies (see Table 6). An association between diagnoses and raw Ct values was observed, where LG IPMNs had overall higher mean Cts across all miRNAs (lower expression) as compared to HG IPMNs (lower mean Ct value, higher expression) (FIG. 5, FIG. 6A and FIG. 6B). This suggests that these miRNAs tend to be higher expressed in more severe disease states.

Singleplex miRNA expression verification of 37 combined “cyst fluid” and “verified tissue miRNAs” (see FIG. 1) was carried out using all CFS1 specimens with sufficient RNA. A reduced set of 13 miRNAs (labeled high priority in Table 6) was investigated in two samples with depleted RNA, Disc-CF2 and -CF4. Using criteria described above, 27 significantly differentially expressed DiffPairs were selected, composed of 18 “verified cyst fluid miRNAs” (FIG. 1 and Table 3). These 18 miRNAs, along with miR-21, were considered the most promising candidates for validation. miR-21 was included in this study based on its reported overexpression and strong association with poor patient outcome in many human cancers (Iorio et al., 2005; Volinia et al., 2006; Chan et al., 2008; Hu et al., 2011; Tetzlaff et al., 2007), including pancreatic (Giovannetti et al., 2010) and IPMNs (Habbe et al., 2009; Dillhoff et al., 2008).

The cyst fluid validation set (CFS2) included 50 specimens collected from patients with histologically confirmed SCAs, LG IPMNs, IG IPMNs, HG IPMNs with and without carcinoma, NETs and SPNs (Table 2). IG IPMNs were initially excluded from the bioinformatics analysis to avoid confounding the separation between LG IPMN and HG IPMN. NETs and SPNs were included as uncommon cysts with malignant or high risk potential for which a resection is usually recommended. The CFS2 data exhibited larger variance in the mean Ct values of SCAs as compared with the CFS1 (FIGS. 6A & 6B). One result of this increased variation was a decreased ability to separate HG IPMN from the LG IPMN/SCA grouping on the basis of mean Ct alone.

Combination of the singleplex RT-qPCR data generated for the verification and validation phases provided a large data set with an opportunity to construct a miRNA-based classifier using a logistic regression with DiffPairs as predictors. In the absence of apparent batch effects between the two sets (FIG. 10), we were able to re-assign 12 CFS2 along with all of the CSF1 specimens to a merged “training set”, with the remainder of the validation set (37 specimens) kept as a “test set” (see Table 2). Because of the greater diagnostic variance in CFS2, a new set of DiffPair predictors was selected for use in the logistic regression model as described in Materials and Methods.

A miRNA-Based Model that Predicts Resection Status.

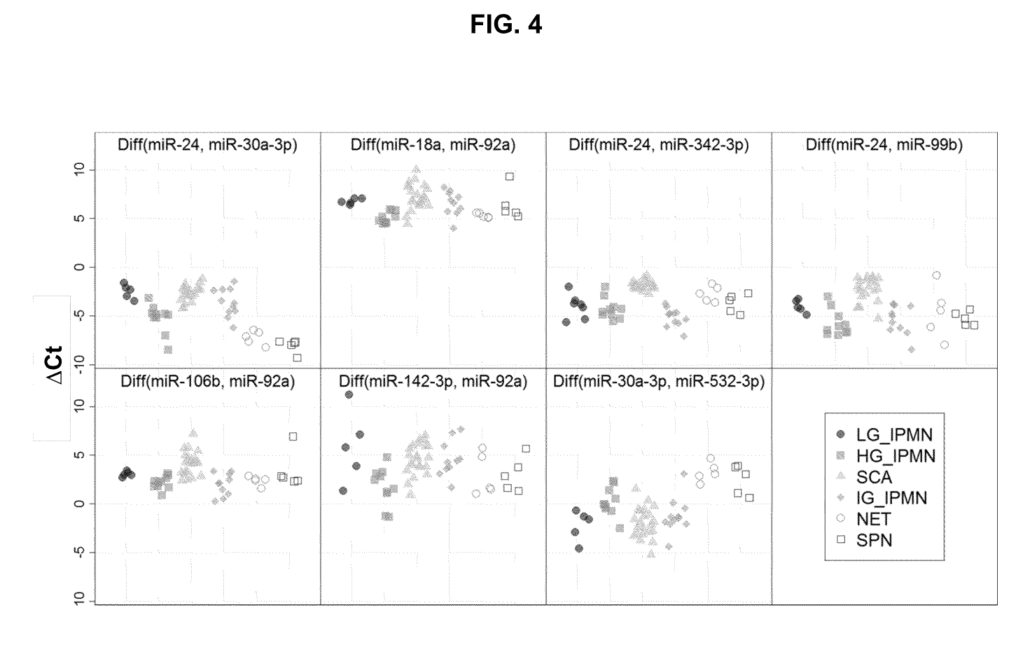

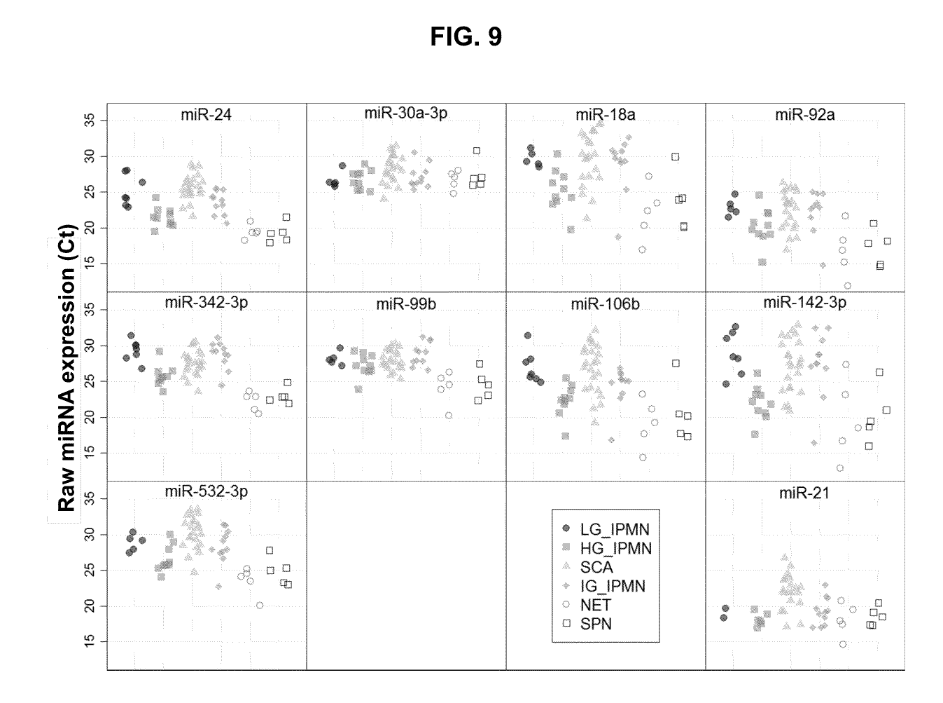

A model to predict resection status was constructed as follows. SCAs were grouped with LG IPMNs since both do not need to be resected unless symptomatic (this group will be referred to as “benign” or “low risk lesion”). HG IPMNs were grouped with SPNs and NETs, due to their high potential for malignancy and common treatment by surgery (this group will be referred to as “malignant” or “high risk lesion”). Because of the L1-penalty applied to the logistic regression weights, only 7 of the 20 predictor Diff Pairs were assigned non-zero weights. The following miRNAs were represented in those DiffPairs: miR-24, miR-30a-3p, miR-18a, miR-92a, miR-342-3p, miR-99b, miR-106b, miR-142-3p and miR-532-3p (Table 4). The ΔCt values associated with these DiffPairs are plotted in FIG. 4 while the raw Ct values associated with the 10 miRNAs used by these DiffPairs are plotted in FIG. 9.

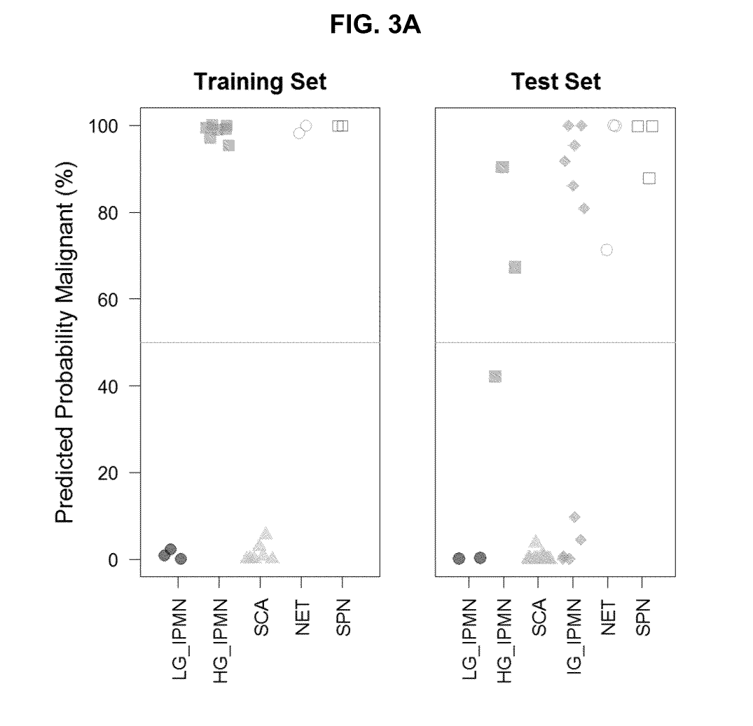

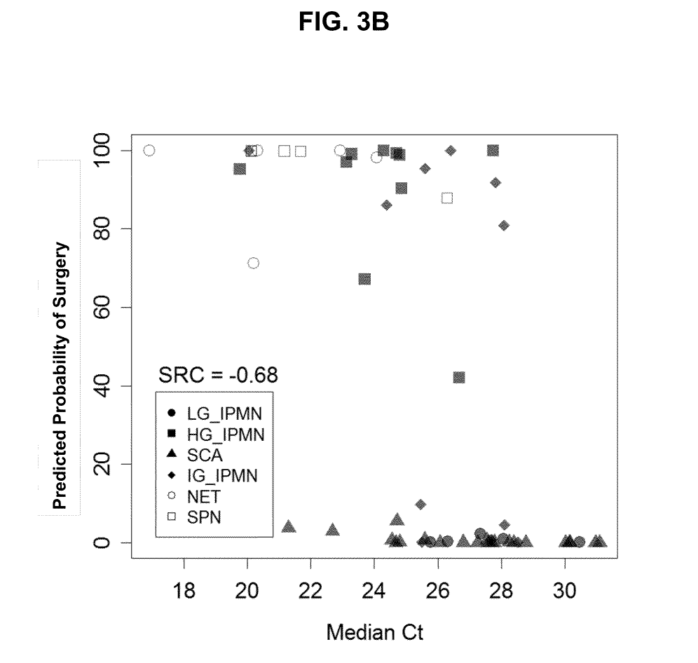

Because the model calculates the probability of being malignant or high risk, the scores for each sample are zero through one inclusive. When the scores were dichotomized using a threshold of 0.5, all SCA/LG IPMN and 90% HG IPMNs were correctly predicted as benign (or low risk) and malignant (or high risk), respectively (FIG. 3A). All SCAs in both the training and the test sets were predicted as benign or low risk. 45.5% IG IPMN specimens (n=5; Valid-CF3, —CF4, —CF10, —CF11, —CF13) were predicted as benign, while the remaining 54.5% (6 specimens; Valid-CF5, —CF6, —CF7, —CF9, —CF12, —CF14) were predicted as malignant or high risk. All SPN and NET samples were predicted to be malignant. Considering the results from the test set (excluding IG IPMNs, for which we do not have independent assessment of malignancy), the sensitivity of this 9-miRNA model was estimated at 89% (95% Wilson interval: 57-98%) and the specificity at 100% (95% Wilson interval: 82-100%) with an AUC of 1. FIG. 3B indicates the extent of correlation of model prediction with median Ct (measured by Spearman Rank Correlation). It is apparent that while median Ct alone has some predictive power, the regression model is capable of stratifying indications far more accurately.

The DiffPair assigned the highest weight in the model, Diff(miR-24, miR-30a-3p), exhibited a pattern of lower ΔCt values for HG IPMNs than for SCAs, LG IPMNs, or (most) IG IPMNs, and even lower ΔCt values for NET and SPN specimens (FIG. 4). The inverse (i.e., DiffPair formed by reversing the order of the constituent miRNAs) of Diff(let-30a-3p, miR-532-3p) shows a comparable pattern to that of Diff(miR-24, miR-30a-3p). Notably, this is the only DiffPair to receive a negative weight in Table 4. This ΔCt pattern suggests that Diff(miR-24, miR-30a-3p) and Diff(miR-30a-3p, miR-532-3p) function in the model to differentiate HG IPMN from benign, but are especially effective at differentiation of NET and SPN from benign or low risk specimens. The ΔCt patterns for Diff(miR-18a, miR-92a), Diff(miR-24, miR-99b), Diff(miR-106b, miR-92a) and Diff(miR-142-3p, miR92a) indicate progressively weaker differentiation of benign (low risk) from malignant (high risk) generally, with less differentiation of ΔCt values between the malignant subgroups (HG IPMN, NET, SPN). These DiffPairs also show generally similar ΔCt values across benign subgroups LG IPMN and SCA, suggesting that they serve as essentially non-specific benign-versus-malignant predictors in the model. The pattern of the third highest-weighted DiffPair, Diff(miR-24, miR-342-3p) is the most unique showing ΔCt values for all IPMN groups much lower than for SCAs, with SPN samples similar to LG IPMNs and NET sample ΔCts between those of SCAs and IPMNs. This DiffPair may thus be used to distinguish SCA samples from all others in model predictions.

The individual 9 miRNAs making up the 7 predictor DiffPairs also showed different expression patterns between experimental groups (FIG. 9). While most of these miRNAs showed lower Ct values (higher expression levels) for HG IPMNs, NETs, and SPNs than for LG IPMNs, SCAs, and (most) IG IPMNs, miR-30a-3p showed little difference between any of the groupings, and appeared to be functioning in the model as a normalizer. Another candidate, miR-99b, appeared to specifically distinguish NETs and SPNs from all IPMNs and SCAs. As for miR-21, it was observed to be down-regulated in SCAs as compared to the remaining diagnostic categories. It did not, however, appear to be a strong predictor of LG IPMN/SCAs vs. HG IPMN, SPNs and NETs.

### Example 2

The following example is related to Example 1 and provides additional embodiments and further analyses of, for example, the use of miRNA biomarkers in cyst fluid to augment the diagnosis and management of pancreatic cysts.

In brief, RNA was extracted from 55 microdissected FFPE IPMN specimens, and 65 cyst fluid (CF) IPMN specimens were aspirated following surgical resection. Expression of 750 miRNAs was evaluated with TaqMan MicroRNA Arrays using 22 FFPE and 15 CF specimens. Differential expression of selected miRNA candidates was validated in 33 FFPE and 50 CF specimens using TaqMan MicroRNA Assays.

In this study, 26 and 37 candidate miRNAs were identified that distinguish low grade (LG) from high grade (HG) IPMNs using FFPE and CF specimens, respectively. A subset of 18 miRNAs, selected from FFPE and CF data, separated HG IPMNs from LG IPMNs, serous cystadenomas (SCAs) and uncommon cysts, such as solid pseudopapillary neoplasms (SPNs) and cystic pancreatic neuroendocrine tumors (PanNETs). A logistic regression model using 9 miRNAs allowed prediction of cyst pathology implying resection (HG IPMNs, PanNETs, SPNs) versus conservative management (LG IPMNs, SCAs), with a sensitivity of 89%, a specificity of 100%, and AUC of 1.

In summary, candidate miRNAs were found that helped identify patients with HG IPMN and exclude non-mucinous cysts.

The following hypothesis was tested—that IPMNs should demonstrate altered miRNA expression profiles that can be detected in the pool of nucleic acids shed into pancreatic CF. It has been shown that miRNAs are stable in different specimen types, including biofluids (Habbe et al., 2009; Szafranska et al., 2008a; Taylor et al., 2008) and that they can be recovered and amplified from these sources. In this Example 2, it is demonstrated that expression patterns of selected miRNAs in CF may indicate the grade of dysplasia [high grade (HG) or low grade (LG)] of an IPMN and thus facilitate therapeutic stratification, and further, may be predictive for other rare cystic lesions that require outright surgical resection (e.g. cystic pancreatic neuroendocrine tumors [cystic PanNETs] and solid pseudopapillary tumors [SPNs]). In short, these biomarkers may be used to facilitate improved management of pancreatic cysts.

### Methods

Patients and biospecimens. Details on patients and biospecimens integrated in this study includes the following. First, this study was approved by the Johns Hopkins University Institutional Review Board. The prospectively maintained Johns Hopkins Surgical Pathology Database was scrutinized to identify formalin-fixed, paraffin-embedded (FFPE) tissue specimens of patients who underwent pancreatectomy for IPMN between Jan. 1, 2000 and Aug. 31, 2010 at Johns Hopkins hospital. Hematoxylin and eosin stained (H+E) reference slides were used to identify samples for subsequent molecular studies.

Histologic diagnoses were reconfirmed by two pathologists according to the latest World Health Organization (WHO) recommendations. IPMNs had to display a papillary epithelium with abundant extracellular mucin and measure per definition >1 cm in maximum diameter. Main duct IPMNs were distinguished from branch duct IPMNs, and a third category of a mixed type was assigned whenever the lesion was located in both main and branch duct. In each case, the final diagnosis referred to the most severe grade of dysplasia observed in the neoplastic epithelium, including low grade (LG), intermediate grade (IG), and high grade (HG) IPMNs. Furthermore, an assessment was made of whether an IPMN had an associated invasive carcinoma.

For unbiased high-throughput (HT) miRNA expression profiling (“FFPE tissue study 1” or “FTS1”), 10 LG IPMNs and 12 HG IPMNs were selected. Seven of the latter had an associated invasive adenocarcinoma. For validation of candidate miRNAs in an independent set of specimens (“FFPE tissue study 2” or “FTS2), an additional 33 archival IPMNs were selected (6 LG IPMNs, 14 HG IPMNs, and 13 HG IPMNs with an associated invasive adenocarcinoma). In summary, 22 FFPE IPMN specimens were selected for unbiased HT miRNA expression profiling (“FFPE tissue study 1″ or “FTS1”) and 33 additional archival IPMNs were selected for “tissue” biomarker validation (“FFPE tissue study 2” or “FTS2”).

Similarly, 15 cyst fluid specimens were selected for HT miRNA expression profiling (“Cyst fluid study 1” or “CFS1”) and an additional 50 cyst fluid samples were selected for “CF” biomarker validation (Cyst fluid study 2″ or “CFS2”). It should be noted, that some specimens were excluded from bioinformatics analyses, based on the insufficient RNA to profile all candidates, failure to amplify greater than 10% of microRNAs, low recovery of miRNA fraction, etc.

MiRNA Expression Analyses in FFPE Tissue and Cyst Fluid Specimens.

High-throughput miRNA expression analyses. Candidate miRNAs that distinguish between high risk lesions (HG IPMNs) and low risk lesions (LG IPMNs, SCA) were identified with a high throughput expression platform, using a panel of 10 LG IPMN and 12 HG IPMN FFPE specimens (FTS1), and an independent set of CF specimens consisting of 4 SCA, 3 LG IPMN and 4 HG IPMN (CFS1). Expression of 750 mature miRNAs (Pool A and B) and 377 mature miRNAs (Pool A) was examined for FTS1 and CFS1, respectively. Briefly, 10 ng of total RNA from each FFPE and CF specimen was added into the Megaplex RT reaction followed by expression analysis using the TaqMan MicroRNA Arrays (Applied Biosystems) as per the manufacturer's protocol.

Singleplex RT-qPCR Verification of miRNA Candidates.

Expression levels of 26 and 37 candidate miRNAs identified in the high-throughput FTS1 and CFS1 studies, respectively, were validated by singleplex RT-qPCR in the same FFPE and cyst fluid specimens, provided that sufficient RNA was present or average amplification indicated sufficient miRNA recovery. Briefly, 10 ng total RNA was used per reverse transcription reaction (30 min, 16° C.; 30 min, 42° C.; 5 min, 85° C.; hold at 4° C.). Positive tissue QC and no-template control (NTC, nucleasefree water) samples were used to control for reagent performance and contamination. qPCR was run on the 7900HT instrument as follows: 10 min at 95° C.; 45 cycles of: 15 sec at 95° C. and 30 sec at 60° C.

Bioinformatics analyses. A novel biomarker discovery approach aiming to identify differentially expressed pairs of miRNA (“DiffPairs”) was used in this study. A DiffPair was generated via subtraction of raw expression of one miRNA from another to generate a self-normalizing biomarker. Evaluating biomarkers as DiffPairs is convenient as it can potentially uncover two anti-correlated miRNAs that in combination have significant power at separating experimental groups of interest (Szafranska et al., 2007).

FFPE tissue study. miRNA candidates from the TaqMan MicroRNA Array platform were selected on the basis of strong Ct estimates (≦30), statistical tests for differential expression (t-test and Wilcox Test, FDR<0.05) and prior indication of their role in pancreatic cancer from literature. Expression of these candidates was verified using singleplex RT-qPCR in the original 22 samples (FTS1) and an additional set of 23 out of 33 FTS2 specimens. Potential batch effects were ruled out prior to the analysis. miRNAs with average expression values >35 Ct across all samples were considered to be non-specifically amplified and therefore were excluded from the final data analysis (Schmittgen et al., 2008). This resulted in identification of 30 DiffPairs comprising 13 miRNAs identified from FTS1 and FTS2 specimens selected for further evaluation in CF specimens (Table 5).

Cyst Fluid Study.

Biomarker candidates for CF specimens were identified through the manual selection and statistical testing for differential expression of individual miRNAs and DiffPair biomarkers using 4 SCA, 3 LG IPMN, and 4 HG IPMN specimens. Only miRNAs with P-values less than 0.01 and DiffPairs with FDR-adjusted P-values less than 0.05 were considered for candidate verification and were combined with the top miRNA candidates identified during the FTS1 and FTS2 studies. Analysis of the resulting 37 miRNAs (Table 6) yielded a shortlist of 18 miRNA candidates that comprised 27 top DiffPairs (Table 3). Expression of these 18 miRNAs together with miR-21 was analyzed in an independent set of 49 out of 50 CFS2 specimens composed of 20 SCAs, 2 LG IPMNs, 11 IG IPMNs, 6 HG IPMNs, 5 PanNETs, and 5 SPNs. The excluded specimen, CFS1-8, was an intermediate grade (IG) IPMN, removed due to insufficient miRNA fraction recovery. miR-21 was included because previous experiments suggested its potential role in pancreatic carcinogenesis (Dillhoff et al., 2008).

Logistic Regression Model to Guide Resection.

The singleplex RT-qPCR expression data for the 18 cyst fluid miRNAs (Table 3) and miR-21 generated from 9 CFS1 with sufficient RNA and 49 CFS2 specimens were merged together and then split into training and test sets. No samples used in the miRNA candidate generation set (CFS1) were included in the test set for establishing model performance. For the purposes of this study, cystic lesions for which surgery is usually the treatment of choice (HG IPMN, cystic PanNET, SPN) were defined as “high risk,” while those potentially managed conservatively (LG IPMN, SCA) were defined as “low risk.” The 20 DiffPairs most differentially expressed between low risk and higher risk specimens in the merged training CF data set were used as predictors for an L1-penalized logistic model.

### Results

miRNA Biomarkers in Microdissected FFPE Specimens.

Since total RNA yield extracted from microdissected FTS1 specimens was as low as 245 ng, multiplex RT and cDNA pre-amplification were used to facilitate expression of 750 mature miRNAs and to preserve RNA material for downstream biomarker verification. The bioinformatics data analysis generated 26 differentially expressed miRNA candidates including: miR-100, -106b, -125b, -139-5p, -145, -150, -151-3p, -17, -196a, -200a, -200b, -20b, -210, -214, -217, -26a, -28-5p, -30a-3p, -30e-3p, -342-3p, -34a, -375, -660, -93, -99a and let-7c. Clear separation between LG and HG IPMN groups was achieved.

Verification of the differential expression of these candidates was carried out in the original FTS1 set and in an independent set composed of 3 LG IPMN, 9 HG IPMN and 11 HG IPMN with associated invasive carcinoma (FTS2). The remaining 10 samples from FTS2 were excluded from further analysis based on overall low miRNA signal or an insufficient amount of RNA to interrogate expression of 26 miRNAs. miRNA candidate ranking analysis of the combined FTS1 and FTS2 biomarke verification data yielded 30 DiffPairs composed of 13 miRNAs for further investigation (Table 5 and Table 6).

Cyst Fluid Specimens.

RNA Yield in Pancreatic CF Specimens. Pancreatic CF specimens with expected lower cellular content, such as SCAs, LG and IG IPMNs, yielded on average 96 ng (range: 8.7-474 ng), 284 ng (range: 13.7-1320 ng) and 312 ng CF (range: 11.6-1456 ng) per 50 μl CF, respectively. Specimens from all remaining diagnostic groups (HG IPMNs −/+invasive carcinoma, PanNETs, SPNs) yielded RNA in excess of 1,400 ng/50 μl CF (range: 52.3-23,666 ng). CF specimens that were “cloudy” in appearance were observed to generate more RNA than those that appeared “watery” (SCAs). Agilent Bioanalyzer analysis of CF RNA showed no distinct 18S and 28S peaks and an average RNA fragment size <100 nucleotides. The adequacy of the CF specimens for miRNA expression profiling was established using miR-103, -191 and -24, which are indicative of the overall miRNA recovery in “compromised” biospecimens (Habbe et al., 2009; Doleshal et al., 2008; Szafranska et al., 2008b].

miRNA Biomarkers in CF Specimens.

High throughput expression profiling of 377 human miRNAs was successfully completed in CF specimens collected from 3 patients with histologically confirmed 4 patients with SCAs, LG IPMNs, 4 patients with HG IPMNs (+/−invasive carcinoma). SCAs were included because they represent cystic lesions that are essentially benign, but are sometimes misdiagnosed as IPMNs (and vice versa) (Wu et al., 2011; Correa-Gallego et al., 2010). Clear separation between experimental groups was observed. Similarly to FFPE specimens, the aim of the bioinformatics analysis was to identify lesions that would be recommended for surgical resection (HG-IPMN) versus those likely to favor conservative management (LG-IPMNs and SCAs). Because only 5 DiffPairs demonstrated P-values <0.05, 5 additional DiffPairs with FDR P-values ranging from 0.05-0.06 on the basis of unadjusted P-value ranking were selected. Of note, an association between diagnoses and raw Ct values was observed, where LG IPMNs showed overall higher mean Cts across all miRNAs (lower expression) as compared to HG IPMNs (lower mean Ct value, higher expression). This may be aresult of a higher proliferation with a higher cell turnover in those more dysplastic lesions.

The bioinformatics analysis of combined FTS(1+2) and CFS1 RT-qPCR data rendered 37 top differentially expressed miRNAs derived from the top 10 CF DiffPairs (17miRNAs) combined with the top ten individual miRNAs (Table 8), the top 13 FFPE tissue miRNAs from the 30 DiffPairs (Table 5) and with six miRNAs (let-7b, miR-223, miR-30b, miR-328, miR-532-3p, miR-590-5p) selected based on high expression levels and their performance as individual candidates and in DiffPairs. Four (let-7c, miR-106b, -342-3p and -93) were shared between the FTS and the CFS analyses (Table 6). Verification of all those candidates was carried out in 9 CFS1 specimens with sufficient RNA, while a reduced set of 13 miRNAs (Table 6) was investigated in the remaining two specimens. Twenty seven significantly differentially expressed DiffPairs composed of 18 miRNAs were selected for validation in an independent set of 38 CF specimens, excluding IG IPMNs (Table 3). One additional candidate, miR-21, was included based on its reported overexpression and strong association with poor patient outcome in many human neoplasms (Iorio et al., 2005; Chan et al., 2008; Volinia et al., 2006; Hu et al., 2011; Tetzlaff et al., 2007), including pancreatic cancer (Giovannetti et al., 2010) and IPMNs (Habbe et al., 2009; Dilhoff et al., 2008). The CFS2 specimen data for SCAs exhibited larger variance in the mean Ct values as compared with the CFS1. One result of this increased variation was a decreased ability to separate HG IPMNs from the LG IPMNs/SCAs grouping on the basis of mean Ct alone.

A miRNA-Based Model that Predicts Resection Status.

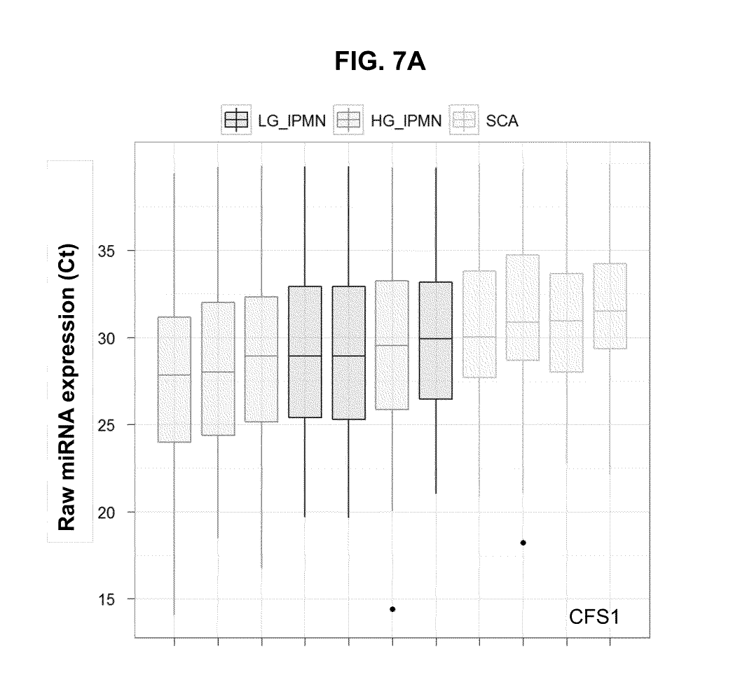

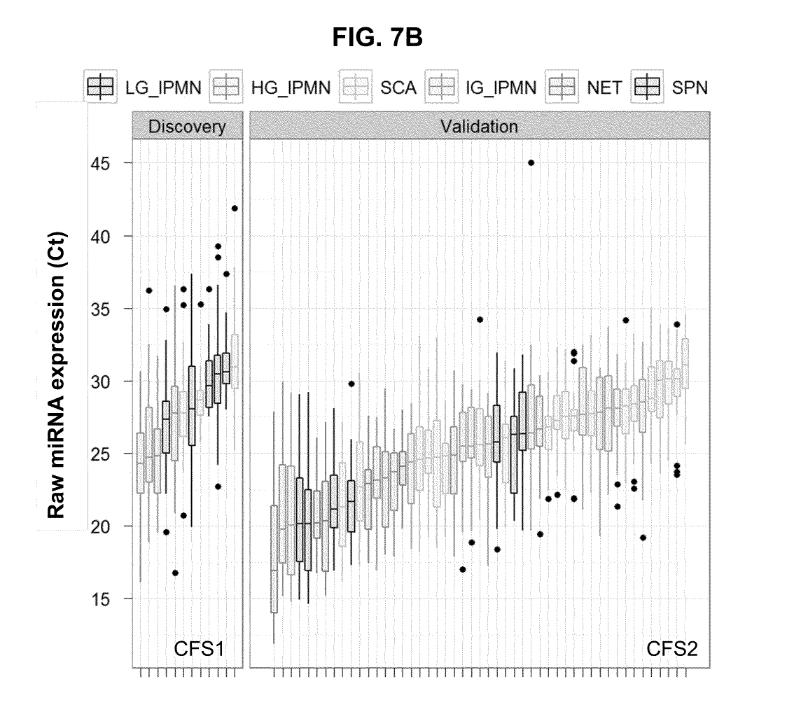

Combination of the singleplex RT-qPCR data for the CFS1 and CFS2 specimens provided an opportunity to construct a miRNA-based classifier to identify high risk lesions, which under the current patient management would be recommended to undergo resection. For simplicity, a classifier was used based on penalized logistic regression with DiffPairs as predictors (Goeman, 2010) and grouped CF specimens into “low risk” (SCAs, LG IPMNs) and “high risk” (HG IPMNs, SPNs and NETs). In order for all diagnostic groups to be represented in the training set, 9 CFS1 specimens were combined with 12 specimens from the CFS2 set to yield a training set containing 7 SCAs, 3 LG IPMNs, 7 HG IPMNs, 2 PanNETs and 2 SPNs, but no IG IPMN. This re-assignment of specimens could be implemented because there were no batch effects observed between the two sets (FIG. 8). Based on the broader diagnostic coverage of the combined training set, a new set of 20 DiffPairs was selected for validation in the remaining 37 CFS2 specimens (test set), including IG IPMNs. Thirteen of those DiffPairs were excluded by the model filtering as they added little in terms of predictive power in differentiating between low and high risk lesions (Table 4). The expression values (ΔCt) for the remaining 7 DiffPairs were used, while the raw Ct values associated with miR-24, -30a-3p, -18a, -92a, -342-3p, -99b, -106b, -142-3p and -532-3p used by these DiffPairs were plotted and analyzed (FIGS. 7A & 7B). The regression coefficients for each of the 7 DiffPairs, which weight each DiffPair in terms of its relevant contribution towards predicting a high risk lesion, are shown in Table 4. The DiffPairs assigned the highest and lowest weights in the model, namely Diff(miR-24, miR-30a-3p) and Diff(miR-30a-3p, miR-532-3p), separated cystic PanNETs and SPNs particularly well, and to a lesser extent HG IPMNs, from LG IPMNs and SCAs. The pattern of the third highest-weighted DiffPair, Diff(miR-24, miR-342-3p), was somewhat unique, showing differentiation of IPMNs in general from SCAs. The ΔCt expression patterns for Diff(miR-18a, miR-92a), Diff(miR-24, miR-99b), Diff(miR-106b, miR-92a) and Diff(miR-142-3p, miR-92a) indicate a progressively weaker differentiation between “low risk” and “high risk” lesions.

Since the model calculates the probability of any given specimen being a “high risk” lesion, the scores for each sample were 0-100% inclusive. When the scores were dichotomized using a threshold of 50% (specimen is “low risk” lesion if the score is less than 50%, and is “high risk” otherwise), all SCA/LG IPMN and 90% HG IPMNs were correctly predicted as “low risk” and “high risk”, respectively. Notably, 3 of the four IPMNs with eventual LG dysplasia (CFS1-1, CFS1-3, CFS1-5), the cyst size was actually greater than 3 cm, thus meeting Sendai high risk criteria for resection. Whether size alone was the clinical rationale for removing the said cysts, our model correctly predicted all three LG IPMNs as “low risk” based on CF analysis, irrespective of cyst size. Conversely, two of the IPMNs with eventual HG dysplasia (CFS2-17 and CFS2-20) were only 1 cm in greatest diameter, but were accurately classified as “high risk” by the 9 miRNA model.

All SCAs in both the training and the test sets were predicted as “low risk”, while all SPN and cystic PanNET samples (typically recommended for surgical resection) were predicted to be “high risk”. Within the cohort of IG IPMNs, 45.5% specimens (n=5) were predicted as “low risk”, while the remaining 54.5% (6 specimens) were predicted as “high risk”; unfortunately in the absence of prospective follow up, the natural history of such lesions remains unclear. Considering the results from the test set (excluding IG IPMNs, for which independent assessment risk was not at hand), the sensitivity of this 9 miRNA model was estimated at 89% (95% Wilson interval: 57-98%) and the specificity at 100% (95% Wilson interval: 82-100%) with an AUC of 1. From the extent of correlation between the prediction model and median Ct (measured by Spearman Rank Correlation) it is apparent that while median Ct alone has some predictive power, the regression model is capable of stratifying indications far more accurately. Of note, miR-21 was not included in our model. In contrast to recent reports (Szafranska et al, 2008b), in this study miR-21 was not among the strongest predictors of a specimen being at low or high risk for therapeutic stratification.

These results underscore the feasibility and advantages of directly profiling the most proximate biological sample to be used for biomarker analyses (in this case, aspirated cyst fluid), rather than extrapolating candidates from tissue profiling experiments alone, as prior studies have done. They show how miRNA biomarkers can be used in diagnosis and surgical treatment decisions for patients with pancreatic cystic lesions, such as HG IPMNs, cystic PanNETs and SPNs.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,384,261
U.S. Pat. No. 5,405,783
U.S. Pat. No. 5,412,087
U.S. Pat. No. 5,424,186
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent application Ser. No. 09/545,207
U.S. patent application Ser. No. 10/667,126
U.S. patent application Ser. No. 11/141,707
U.S. patent application Ser. No. 11/273,640
U.S. Patent Prov. Appn. Ser. No. 60/575,743
U.S. Patent Prov. Appn. Ser. No. 60/649,584
U.S. Patent Prov. Appn. Ser. No. 60/826,173
Akao et al., DNA Cell Biol., 26:311-320, 2007.
Ali et al., Am. J. Trans'. Res., 3:28-47, 2010.
Allen et al., Ann. Surg., 250:754-760, 2009.
Ambros, Cell, 107(7):823-826, 2001.
Beaucage, and Lyer, Tetrahedron, 48:2223-2311, 1992.
Brennecke et al., Cell, 113:25-36, 2003.
Calin et al., Proc. Natl. Acad. Sci. USA, 99:15524-15529, 2002.
Carrington et al. Science, 301(5631):336-338, 2003.
Chan et al., Anticancer Res., 28:907-911, 2008.
Chen et al., BMC Genomics, 10:407, 2009.
Cummins et al., In: IRT: Nucleosides and nucleosides, La Jolla Calif., 72, 1996.
Denli et al., Trends Biochem. Sci., 28:196, 2003.
Didenko, Biotechniques, 31(5):1106-16, 1118, 1120-1, 2001.
Dillhoff et al., J. Gastrointest. Surg., 12:2171-2176, 2008.
DiMagno, Clin. Gastroenterol. Hepato'., 5:797-798, 2007.
Doleshal et al., J. Mol. Diagn., 10:203-211, 2008.
Emptage et al., Neuron, 2001 January; 29(1):197-208, 2001.
EP 266,032
EP 373 203
EP 785 280
EP 799 897
Esquela-Kerscher and Slack, Nat Rev Cancer, 6(4):259-269, 2006.
Fodor et al., Science, 251:767-777, 1991.
Freelove and Walling, Am. Fam. Physician, 73(3):485-492, 2006.
Froehler et al., Nucleic Acids Res., 14(13):5399-5407, 1986.
Gillam et al., J. Biol. Chem., 253:2532, 1978.
Gillam et al., Nucleic Acids Res., 6:2973, 1979.
Giovannetti et al., Cancer Res., 70:4528-4538, 2010.
Gironella et al., Proc. Natl. Acad. Sci. USA, 104:16170-16175, 2007.
Gottardo et al., Urologic Oncol. Sem. Orig. Invest., 25:387-392, 2007.
Griffey et al., J Mass Spectrom, 32(3):305-13, 1997.
Habbe et al., Cancer Biol. Ther., 8:340-346, 2009.
He et al., Proc. Natl. Acad. Sci. USA, 102(52):19075-19080, 2005.
Hu et al., Int. J. Cancer, 128:132-143, 2011.
Iorio et al., Cancer Res., 65:7065-7070, 2005.
Itakura and Riggs, Science, 209:1401-1405, 1980.
Itakura et al., J. Biol. Chem., 250:4592, 1975.
Jemal et al., CA Cancer J. Clin., 56(2):106-130, 2006.
Jemal et al., CA Cancer J. Clin., 60:277-300, 2010.
Khalid et al., Clin. Gastroenterol. Hepatol., 3:967-973, 2005.
Khalid et al., Gastrointest. Endosc., 69:1095-1102, 2009.
Khorana, Science, 203, 614, 1979.
Kimura et al., Int. J. Pancreatol., 18:197-206, 1995.
Klostermeier and Millar, Biopolymers, 61(3):159-79, 2001-2002.
Kornberg and Baker, In: DNA Replication, 2d Ed., Freeman, San Francisco, 1992.
Lagos-Quintana et al., Science, 294(5543):853-858, 2001.
Lanza et al., Mol. Cancer, 6:54, 2007.
Lau et al., Science, 294(5543):858-862, 2001.
Lee and Ambros, Science, 294(5543):862-864, 2001.
Lee et al., EMBO J. 21:4663-70, 2002.
Lu et al., Nature, 435(7043):834-838, 2005.
Matthaei and Maitra, In: Pre-Invasive Disease:Pathogenesis and Clinical Management, Fitzgerald (Ed.), Springer, N.Y., 395-420, 2011.
Mestdagh et al., Nucleic Acids Res., 36:e143, 2008.
Michael et al., Mol. Cancer. Res., 1:882-891, 2003.
Monti et al., Virchows Arch., 445(3):236-247, 2004.
Nagula et al., J. Gastrointest. Surg., 14:1997-2003, 2010.
Olsen et al., Dev. Biol., 216:671, 1999.
Raval et al., Mod. Pathol., 23:1467-1476, 2010.
Roggin et al., Curr. Probl. Surg., 47:459-510, 2010.
Ryu et al., Diagn. Cytopathol., 31:100-105, 2004.
Ryu et al., Pancreatology, 10:66-73, 2010.
Ryu et al., Pancreatology, 11:343-350, 2011.
Sambrook et al., In: DNA microaarays: a molecular cloning manual, Cold Spring Harbor Laboratory Press, Cold Spring Harbor, N.Y., 2003.
Sambrook et al., In: Molecular cloning: a laboratory manual, 2^{nd} Ed., Cold Spring Harbor Laboratory Press, Cold Spring Harbor, N.Y., 1989.
Sambrook et al., In: Molecular cloning: a laboratory manual, 3^{rd} Ed., Cold Spring Harbor Laboratory Press, Cold Spring Harbor, N.Y., 2001.
Schmittgen et al., Methods, 44:31-38, 2008.
Schoedel et al., Diagn. Cytopathol., 34:605-608, 2006.
Seggerson et al., Dev. Biol., 243:215, 2002.
Szafranska et al., J. Mol. Diagn., 10:415-423, 2008.
Tanaka et al., Pancreatology, 6:17-32, 2006.
Tanaka, Nat. Rev. Gastroenterol. Hepatol., 8:56-60, 2011.
Tetzlaff et al., Endocr. Pathol., 18:163-173, 2007.
U.K. Patent 1,529,202
UK Appln. 8 803 000
Volinia et al., Proc. Natl. Acad. Sci. USA, 103:2257-2261, 2006.
Winter et al., J. Gastrointest. Surg., 10:1199-1210, 2006.
WO 0138580
WO 0168255
WO 03020898
WO 03022421
WO 03023058
WO 03029485
WO 03040410
WO 03053586
WO 03066906
WO 03067217
WO 03076928
WO 03087297
WO 03091426
WO 03093810
WO 03100012
WO 03100448A1
WO 04020085
WO 04027093
WO 09923256
WO 09936760
WO 93/17126
WO 95/11995
WO 95/21265
WO 95/21944
WO 95/35505
WO 96/31622
WO 97/10365
WO 97/27317
WO 9743450
WO 99/35505
World Health Organization recommendations (WHO), Bosman et al., WHO Classification of Tumours of the Digestive System IARC. Lyon: 2010.
Wu et al., Sci. Trans'. Med., 3:92ra66, 2011.
Xu et al., Curr. Biol., 13:790-795, 2003.
Yeo et al., Curr Prob Cancer, 26:176-275, 2002.

1. A method for evaluating a pancreatic cyst in a patient comprising:
a) measuring from a pancreatic cyst sample from the patient the level of expression of at least two of the following biomarker miRNAs: miR-24, miR-30a-3p, miR-92a, miR-18a, miR-342-3p, miR-99b, miR-106b, miR-142-3p, or miR-532-3;
b) comparing the level of expression each biomarker miRNA to the level of expression of another biomarker miRNA;
c) calculating a diagnostic score that indicates the probability the pancreatic cyst is a low risk or high risk lesion, wherein the diagnostic score is based on comparisons between the expression levels of the biomarker miRNAs to the expression level of at least one other biomarker miRNA.

2.-14. (canceled)

15. The method of claim 1, wherein the pancreas sample is a cystic fluid sample.

16. The method of claim 15, wherein the sample is obtained by fine needle aspirate.

17.-23. (canceled)

24. The method of claim 16, further comprising determining diffpair values after measuring and comparing the level of expression of miRNAs, where the diff pair values are calculated from at least two of the following diffpairs: miR-24/miR-30a-3p; miR-18a/miR-92a; miR-24/miR-342-3p; miR-24/miR-99b; miR-106b/miR-92a; miR-142-3p/miR-92a; or miR-30a-3p/miR-532-3p.

25.-29. (canceled)

30. The method of claim 24, further comprising measuring the level of expression of at least one of the following miRNAs: miR-15a, miR-16, miR-21, miR-17-5p, miR-100, miR-107, miR-155, miR-181a, miR-181c, miR-210, miR-221, or miR-223.

31.-32. (canceled)

33. The method of claim 1, wherein the diagnostic score is based on determined diff pair values.

34.-36. (canceled)

37. The method of claim 1, further comprising resecting all or part of a pancreatic cyst determined to be a high risk lesion.

38.-45. (canceled)

46. A method of evaluating a pancreatic cyst from a patient comprising:
a) from a sample of the pancreatic cyst, measuring the level of expression of at least miR-24 and at least one of the following comparative microRNAs: miR-30a-3p, miR-342-3p, and miR-99b;
b) comparing the level of expression between miR-24 and the at least one comparative microRNA to determine a mitt-24 diffpair value; and,
c) calculating a diagnostic score that indicates whether the pancreatic cyst is a low risk or high risk lesion.

47.-51. (canceled)

52. The method of claim 46, wherein the sample is a cystic fluid sample.

53. The method of claim 46, wherein the cystic fluid sample is obtained by fine needle aspirate.

54. The method of claim 46, further comprising measuring the sample the level of expression of at least one of the following additional microRNAs: miR-18a, miR-30a-3p, miR-92a, miR-99b, miR-106b, miR-142-3p, miR-342-3p, or miR-532-3p.

55.-61. (canceled)

62. The method of claim 46, further comprising determining diffpair values after measuring and comparing the level of expression of miRNAs from at least two of the following diffpairs: miR-24/miR-30a-3p; miR-18a/miR-92a; miR-24/miR-342-3p; miR-24/miR-99b; miR-106b/miR-92a; 42-3p/miR-92a; or miR-30a-3p/miR-532-3p.

63.-67. (canceled)

68. The method of claim 46, further comprising measuring the level of expression of at least one of the following miRNAs: miR-15a, miR-16, miR-21, miR017-5p, miR100, miR-107, miR-155, miR-181a, miR-181c, miR-210, miR-221, or miR-223.

69.-73. (canceled)

74. The method of claim 46, further comprising determining a treatment for the patient based on a calculated diagnostic score.

75. The method of claim 46, further comprising resecting all or part of the high risk lesion.

76.-92. (canceled)

93. A method for analyzing a pancreatic cyst sample from a patient comprising:
a) measuring the level of expression in the cyst sample at least two of the following biomarker miRNAs: miR-18a, miR-24, miR-30a-3p, miR-92a, miR-99b, miR-106b, miR-142-3p, miR-342-3p, miR-532-3p;
b) measuring the level of expression of at least one comparative miRNA, wherein the level of expression of the comparative miRNA is compared with one or more biomarker miRNA expression levels;
c) comparing each of the at least two levels ofbiomarker expression with the level of expression of the at least one comparative RNA to determine a diff pair value; and,
d) determining a diagnostic score that indicates the risk for a high risk lesion or a low risk lesion.

94.-119. (canceled)

120. A method for evaluating a pancreatic cyst in a patient comprising:
a) measuring from a pancreatic cyst sample from the patient the level of expression of at least one of the following miRNAs: miR-18a, miR-24, miR-30a-3p, miR-92a, miR-99b, miR-106b, miR-142-3p, miR-342-3p, or miR-532-3p;
b) comparing the level of expression of the at least one miRNA to the level of expression of at least a first comparative microRNA; and,
c) evaluating the sample based on the comparison between the level of expression of the at least one miRNA to the level of expression of at least a first comparative microRNA, wherein the evaluation indicates whether the pancreatic cyst is a low risk or high risk lesion.

121.-129. (canceled)

130. A tangible computer-readable medium comprising computer-readable code that, when executed by a computer, causes the computer to perform operations comprising:
a) receiving information corresponding to a level of expression in a pancreatic cyst sample from a patient of at least two of the following diffpair miRNAs: miR-18a, miR-24, miR-30a-3p, milt-92a, miR-99b, miR-106b, miR-142-3p, miR-342-3p, or miR-532-3p, wherein at least one of the miRNAs is a biomarker miRNA; and
b) determining a biomarker diff pair value using information corresponding to the at least one biomarker miRNA and information corresponding to the level of expression of a comparative micro RNA, the diff pair value being indicative of whether the pancreatic cyst is a low risk or high risk lesion.

131.-135. (canceled)


SEQUENCE LISTING (9 sequences)

SEQ ID NO: 1 - RNA, 23 nt, Homo sapiens
uaaggugcaucuagugcagauag

SEQ ID NO: 2 - RNA, 22 nt, Homo sapiens
uggcucaguucagcaggaacag

SEQ ID NO: 3 - RNA, 22 nt, Homo sapiens
cuuucagucggauguuugcagc

SEQ ID NO: 4 - RNA, 22 nt, Homo sapiens
uauugcacuugucccggccugu

SEQ ID NO: 5 - RNA, 22 nt, Homo sapiens
cacccguagaaccgaccuugcg

SEQ ID NO: 6 - RNA, 21 nt, Homo sapiens
uaaagugcugacagugcagau

SEQ ID NO: 7 - RNA, 23 nt, Homo sapiens
uguaguguuuccuacuuuaugga

SEQ ID NO: 8 - RNA, 23 nt, Homo sapiens
ucucacacagaaaucgcacccgu

SEQ ID NO: 9 - RNA, 22 nt, Homo sapiens
ccucccacacccaaggcuugca